SEQUENCE LISTING (39 sequences)

SEQ ID NO: 1 - DNA, 40 bp, Artificial Sequence
  Chemically synthesized oligonucleotide
gagattggtaaactcctcaactcgtatctggaaaagaaaa

SEQ ID NO: 2 - DNA, 40 bp, Artificial Sequence
  Chemically synthesized oligonucleotide
cagatacgagttgaggagtttaccaatctccgttgatctt

SEQ ID NO: 3 - DNA, 30 bp, Artificial Sequence
  Chemically synthesized oligonucleotide
cagtggaaaaaaagctgcatgaatcaggtt

SEQ ID NO: 4 - DNA, 30 bp, Artificial Sequence
  Chemically synthesized oligonucleotide
atgcagctttttttccactgatagcccaac

SEQ ID NO: 5 - DNA, 18 bp, Artificial Sequence
  Chemically synthesized oligonucleotide
gtgctggagggtgtggac

SEQ ID NO: 6 - DNA, 20 bp, Artificial Sequence
  Chemically synthesized oligonucleotide
ttcagaagcttgccgatttc

SEQ ID NO: 7 - DNA, 20 bp, Artificial Sequence
  Chemically synthesized oligonucleotide
ctgggatttggtgcaaactt

SEQ ID NO: 8 - DNA, 20 bp, Artificial Sequence
  Chemically synthesized oligonucleotide
gcccgcttcatgtctacaat

SEQ ID NO: 9 - DNA, 20 bp, Artificial Sequence
  Chemically synthesized oligonucleotide
ctaaggccaaccgtgaaaag

SEQ ID NO: 10 - DNA, 20 bp, Artificial Sequence
  Chemically synthesized oligonucleotide
gaccagaggcatacagggac

SEQ ID NO: 11 - DNA, 21 bp, Artificial Sequence
  Chemically synthesized oligonucleotide
caagagatggccagggctgct

SEQ ID NO: 12 - DNA, 21 bp, Artificial Sequence
  Chemically synthesized oligonucleotide
tccttctgcatcctgtcggca

SEQ ID NO: 13 - DNA, 21 bp, Artificial Sequence
  Chemically synthesized oligonucleotide
caagcttctgaattcctactt

SEQ ID NO: 14 - DNA, 21 bp, Artificial Sequence
  Chemically synthesized oligonucleotide
cttctctgcaaaccgctggga

SEQ ID NO: 15 - DNA, 21 bp, Artificial Sequence
  Chemically synthesized oligonucleotide
acggaactagaggatcactct

SEQ ID NO: 16 - DNA, 21 bp, Artificial Sequence
  Chemically synthesized oligonucleotide
gaaatcggcaagcttctgaat

SEQ ID NO: 17 - DNA, 21 bp, Artificial Sequence
  Chemically synthesized oligonucleotide
caagaccacgcagggcctcaa

SEQ ID NO: 18 - DNA, 21 bp, Artificial Sequence
  Chemically synthesized oligonucleotide
gctgtgaatagaataactgaa

SEQ ID NO: 19 - DNA, 21 bp, Artificial Sequence
  Chemically synthesized oligonucleotide
gccacgagaatgtagtgaaat

SEQ ID NO: 20 - DNA, 21 bp, Artificial Sequence
  Chemically synthesized oligonucleotide
cccatgtagtagtatcacttt

SEQ ID NO: 21 - DNA, 21 bp, Artificial Sequence
  Chemically synthesized oligonucleotide
gtggaagaagagttgtatgaa

SEQ ID NO: 22 - DNA, 21 bp, Artificial Sequence
  Chemically synthesized oligonucleotide
gcaacggtatttcggcataat

SEQ ID NO: 23 - DNA, 21 bp, Artificial Sequence
  Chemically synthesized oligonucleotide
gtcctgttcctccagttacag

SEQ ID NO: 24 - DNA, 21 bp, Artificial Sequence
  Chemically synthesized oligonucleotide
gaaatcggcaagcttctgaat

SEQ ID NO: 25 - DNA, 21 bp, Artificial Sequence
  Chemically synthesized oligonucleotide
caagaccacgcagggcctcaa

SEQ ID NO: 26 - DNA, 21 bp, Artificial Sequence
  Chemically synthesized oligonucleotide
gcgatcacatggtcctgctgg

SEQ ID NO: 27 - DNA, 23 bp, Artificial Sequence
  Chemically synthesized oligonucleotide
ttctgaccaccaccaacttcaat

SEQ ID NO: 28 - DNA, 22 bp, Artificial Sequence
  Chemically synthesized oligonucleotide
ctaagccggcactccttacaag

SEQ ID NO: 29 - DNA, 20 bp, Artificial Sequence
  Chemically synthesized oligonucleotide
ctagccaccatggcttgagt

SEQ ID NO: 30 - DNA, 23 bp, Artificial Sequence
  Chemically synthesized oligonucleotide
tccgaattcagtgactacagatg

SEQ ID NO: 31 - DNA, 20 bp, Artificial Sequence
  Chemically synthesized oligonucleotide
agcacataggaggcagagac

SEQ ID NO: 32 - DNA, 21 bp, Artificial Sequence
  Chemically synthesized oligonucleotide
cacaaaaacaggttaaaccca

SEQ ID NO: 33 - DNA, 21 bp, Artificial Sequence
  Chemically synthesized oligonucleotide
cacaaaaacaggttaacccag

SEQ ID NO: 34 - DNA, 20 bp, Artificial Sequence
  Chemically synthesized oligonucleotide
gaagacagaaaaggggaggg

SEQ ID NO: 35 - DNA, 30 bp, Artificial Sequence
  Chemically synthesized oligonucleotide
tctaacaatgcgctcatcgtcatcctcggc

SEQ ID NO: 36 - DNA, 24 bp, Artificial Sequence
  Chemically synthesized oligonucleotide
gagatgggtaccaggagttggggc

SEQ ID NO: 37 - DNA, 25 bp, Artificial Sequence
  Chemically synthesized oligonucleotide
gggcttccacctggtgccagcctgt

SEQ ID NO: 38 - DNA, 25 bp, Artificial Sequence
  Chemically synthesized oligonucleotide
gatggaggacctcttggccggctca

SEQ ID NO: 39 - DNA, 24 bp, Artificial Sequence
  Chemically synthesized oligonucleotide
gagatgggtaccaggagttggggc

(19) United States
(12) Patent Application Publication
(10) Pub. No.: US 2014/0314791 A1
(43) Pub. Date: Oct. 23, 2014

(54) METHODS OF TREATING CANCER

(71) Applicant: DANA-FARBER CANCER INSTITUTE, INC., Boston, MA (US)

(72) Inventors: Kwok-Kin Wong, Arlington, MA (US); Yan Liu, Boston, MA (US)

(21) Appl. No.: 14/370,358

(22) PCT Filed: Jan. 4, 2013
(86) PCT No.: PCT/US2013/020310

§ 371 Date: Jul. 2, 2014

Related U.S. Application Data
(60) Provisional application No. 61/583,362, filed on Jan. 5, 2012.

(51) Int. Cl. C12N 15/113; A61K 45/06; A61K 39/395; A61K 31/55; G01N 33/50; A61K 31/407; A61K 31/553; A61K 31/497; A61K 31/415; C07K 16/40; A61K 31/4535

(52) U.S. Cl. CPC C12N 15/1137; C07K 16/40; A61K 45/06; A61K 39/3955; A61K 31/55; A61K 31/4535; A61K 31/407; A61K 31/553; A61K 31/497; A61K 31/415; G01N 33/5011
USPC 424/158.1; 514/44 A; 514/212.06; 514/326; 514/410; 514/211.08; 514/255.05; 514/407; 506/10; 435/325

(57) ABSTRACT

The present invention provides methods of treating cancer, particularly cancers that are null or have decreased expression or activity of the Lkb1 gene. Also included are methods of identifying therapeutic targets for the treatment of cancer.

### RELATED APPLICATIONS

This application claims priority to and benefit of provisional application U.S. Ser. No. 61/583,362 filed on Jan. 5, 2012, the contents of which are herein incorporated by reference in its entirety.

### INCORPORATION OF SEQUENCE LISTING

The contents of the text file named “20363-063001WO_ST25.txt,” which was created on Dec. 28, 2012 and is 8.3 KB in size, are hereby incorporated by reference in their entirety.

### FIELD OF THE INVENTION

The present invention relates generally to treating cancer. Also included are methods of identifying therapeutic targets for the treatment of cancer.

### BACKGROUND OF THE INVENTION

LKB1 was discovered in 1998 as the gene mutated in Peutz-Jeghers Syndrome, a hereditary, autosomal dominant condition characterized by hamartomatous polyps of the gastrointestinal tract and a larger than 15-fold elevated overall cancer risk (Hearle et al., 2006; Hemminki et al., 1998). In recent years, extensive cancer genetic studies have shown that LKB1 is a major tumor suppressor frequently inactivated in many common types of cancer, including non-small cell lung cancer (NSCLC), where somatic inactivation is seen in 25-30% of NSCLC (Ding et al., 2008; Ji et al., 2007).

Activating KRAS mutations are also common in NSCLC, with a 20-30% frequency in adenocarcinoma of the lung (Ding et al., 2008). Concurrent KRAS activating and LKB1 inactivating mutations are also relatively common in NSCLC, seen in 10-15% of patients (Makowski and Hayes, 2008; Matsumoto et al., 2007). We have previously shown that Lkb1 loss acts synergistically with Kras activation to markedly accelerate lung tumor development and metastasis in a genetically engineered mouse model (GEMM), in comparison to mice harboring Kras activation mutation alone (Ji et al., 2007). Another commonly co-mutated gene in NSCLC is TP53, with an overall mutation rate of ˜50% of NSCLC (Mogi and Kuwano, 2011).

LKB1 encodes serine/threonine kinase 11 (also termed STK11) and is a master regulator of cell metabolism via its interaction with AMPK (Jansen et al., 2009; Shah et al., 2008). LKB1 phosphorylates and activates AMPK in response to low cellular ATP levels. One of the major targets of LKB1-AMPK signaling is the mTOR complex 1 (mTORC1), a key nutrient sensor that promotes cell growth when nutrients are plentiful. AMPK inhibits mTORC1 both indirectly through phosphorylation of TSC2 which results in inhibition of the small GTP-binding protein RHEB, thereby reducing activation of mTORC1 (Jansen et al., 2009; Shah et al., 2008), and directly via phosphorylation and inactivation of the mTOR binding partner Raptor (Kim et al., 2011). AMPK also acts in an mTOR-independent fashion to reprogram cellular metabolism through phosphorylation of targets involved in fatty acid synthesis, glucose uptake, and metabolic gene expression. Therefore, LKB1 signaling is critical for energy sensing and energy stress response, with the LKB1-AMPK pathway playing critical roles in conserving cellular ATP levels through activation of catabolic pathways and switching off ATP-consumptive processes such as macromolecular biosynthesis (Hardie, 2007). In addition, LKB1 activates a family of AMPK-related kinases, many of which are implicated in cellular metabolism, such as the SIK1 and SIK2 kinases (Mihaylova and Shaw, 2011). Consistent with key in vivo roles of additional targets of LKB1 in regulation of metabolism, it was recently reported that LKB1-deficient hematopoietic stem cells exhibit AMPK-independent alterations in lipid and nucleotide metabolism as well as depletion of cellular ATP (Gurumurthy et al., 2010). Overall, LKB1 deficiency results in broad defects in metabolic control, as evidenced by primary cells and cancer cell lines lacking LKB1 being sensitized to nutrient deprivation and other metabolic stress. Thus, there is considerable interest in targeting metabolism as a novel therapeutic strategy in LKB1 mutant cancers.

There is an immediate, critical need for improved therapies for LKB1 mutant cancers due to their prevalence and aggressiveness. Currently, few drugs are available for clinical use that target loss of LKB1 in a specific fashion. mTORC1 inhibitors, such as sirolimus and temsirolimus, have been used with limited success in LKB1 mutant cancers (Faivre et al., 2006). Since these drugs do not inhibit all of the effects of LKB1 loss and are counteracted by feedback, this is not surprising. In addition, tumors harboring KRAS activating mutations have also shown a poor response to conventional chemotherapy, with or without concurrent LKB1 inactivation. Thus a need exists for the identification of therapeutic compounds useful in treating LKB1 null cancers.

### SUMMARY OF THE INVENTION

In one aspect the invention provides methods of treating a subject having a Lkb1 null cancer by administering to the subject a compound that inhibits the expression of activity of deoxyihymidylate kinase (DTYMK), checkpoint kinase 1 (CHEK1) or both. The cancer is for example, lung cancer, melanoma, pancreatic cancer, endometrial cancer, or ovarian cancer. The compound is a nucleic acid, an antibody or a small molecule. In one embodiment the compound is a CHEK1 inhibitor. CHEK 1 inhibitors include for example, AZD7762, Go-6976, UCN-01, CCT244747, TCS2312, PD 407824, PF 477736, PD-321852, SB218078, LY2603618, LY2606368, CEP-3891, SAR-020106, debromohymenialdisine, or CHIR24. Optionally the subject is further administered a chemotherapeutic agent such as a tyrosine kinase inhibitor or an mTOR inhibitor.

In another aspect the invention provides methods of screening for therapeutic targets for treating cancer by providing a cell that is null for a Lkb1 gene, an ATM gene, a TSC1 gene, a PTEN gene or a Notch gene; contacting the cell with a library of RNAi; and identifying an RNAi which is lethal to the cell.

In a further aspect the invention provides methods of treating an ATM, a TSC1, a PTEN or a Notch null cancer by administering a compound that inhibits the expression or activity of the therapeutic target identified by the methods of the invention. The therapeutic target is, for example, DTYMK, CHEK1 or both.

The invention provides a cell expressing KRAS G12D and comprising a disruption of the Trp53 gene, the Lkb1 gene or both, wherein the disruption results in decreased expression or activity of Trp53 gene, the Lkb1 gene or both in the cell. In some embodiments, the cell is a cancer cell, for example a lung cancer cell, a melanoma cancer cell, a pancreatic cancer cell, an endometrial cancer cell or an ovarian cancer cell.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTIONS OF THE DRAWINGS

FIG. 1. Pooled shRNA screening

(A) Unsupervised hierarchical clustering analysis of results from triplicate pooled shRNA library screens of Lkb1-wt and Lkb1-null mouse cancer cell lines based upon log 2 fold change (log 2FC). Negative numbers reflect relative depletion of shRNAs at late time points.

(B) Two class comparison of Lkb1-null versus Lkb1-wt cell lines were used to generate a ranked hairpin list of selectively essential hairpins in an Lkb1-null background. Hairpins were collapsed to gene values using either the weighted second best or the KS statistic in GENE-E. Venn diagram depicts the overlap of most essential genes in the Lkb1-null background nominated by the top 100 independent hairpins, and the top 200 genes from both weighted second best and KS.

(C) Validation study. Relative viability of Lkb1-wt and Lkb1-null cells infected with 340 individual hairpins for 5 days. Genes of interest are highlighted by the colors indicated.

FIG. 2. Kinase inhibitor screening and metabolite profiling

(A) A high-throughput kinase inhibitor screen. Lkb1-wt and Lkb1-null cells were treated with a collection of 998 kinase inhibitors for 2 days, and live cells were monitored with the promega CellTiter-Glo Luminescent Cell Viability assay. The heatmap on the left represents the results of unsupervised cluster analysis of cell growth relative to DMSO-treated cells. The heatmap on the right provides an expanded view of the compounds with greatest activity in this assay, as well as the names of the compounds, the scores (representing the ratio of growth of Lkb1-wt to Lkb1-null), and p-values for differences between the Lkb1-wt and Lkb1-null cell lines (Student's t-test).

(B) Metabolic signature of Lkb1-null lung cancer cells. Unsupervised clustering analysis of metabolic data from Lkb1-wt and Lkb1-null cells. The heatmap displays those metabolites with the greatest difference between Lkb1-wt and Lkb1-null cell lines, along with compound name (ID), Description (KEGG identification number), and p-value, etc. for the comparison between the two sets of lines. The lower panel shows significantly enriched metabolic pathways in down-regulated components of the Lkb1-null metabolic signature using Pathway Analysis module from MetaboAnalist tool (http://www.metaboanalyst.ca).

(C) A comprehensive metabolic map of de novo (solid line) and the salvage (dashed line) pyrimidine deoxyribonucleotide biosynthetic pathway. This map was created with CellDesigner version 4.2 using a template from Panther Classification System Database (www.pantherdb.org). DTYMK is highlighted in Bold. Metabolites CDP, dCDP, UDP and dTDP were significantly down-regulated, and UTP was significantly up-regulated in Lkb1-null cells.

FIG. 3. In vitro and in vivo proliferation assays

(A) Western blot analysis of DTYMK and CHEK1 expression in Lkb1-wt 634 cells upon knockdown of Dtymk or Chek1 with the indicated shRNAs.

(B) Lkb1-wt (634, 855, and 857) and Lkb1-null (t2, t4, and t5) cells were transduced with the indicated shRNA for 2 days and then plated into 96-well plates at 2000 cells/well in 100 μl medium with 3 μg/ml puromycin (puro). Viable cells were measured daily using Promega's CellTiter-Glo Assay. Two independent sets of transductions into the 6 cell lines were shown: the first set used shGFP, shDtymk-1, and shChek1-4 (upper panels), and the second set used shGFP, shDtymk-3, and shChek1-1 (lower panels). The data represent mean±SD for 3 replicates.

(C) 1×10⁶ Lkb1-wt (634 and 857) and Lkb1-null (t2 and t4) cells transduced with the indicated shRNA were implanted into athymic nude mice for 3 weeks. Tumor volume (mm³) was calculated as (length×width)/2. The data represent mean±SD for 4 mice. Lkb1-wt 634 and Lkb1-null t4 tumors with the indicated shRNAs were shown (D).

(E) Lkb1-null t4 cells were first transduced with pCDH-Dtymk(R) or pCDH-Chek1(R) vector co-express GFP, and t4-Dtymk(R) and t4-Chek1(R) cells were sorted by FACS for GFP. The t4-Dtymk(R) and t4-Chek1(R) cells were further transduced with shGFP, shDtymk-3, or shChek1-4, and then plated into 96-well plates for proliferation as in (B).

FIG. 4. dTTP rescues shDtymk growth phenotype

(A) Graph of dTMP and dTDP levels in Lkb1-wt 634, Lkb1-null t4, and human LKB1-deficient NSCLC A549 cells transduced with the indicated shRNA for 3 days. The data represent mean±SD for 6 replicates.

(B) Morphology of Lkb1-null t2, t4, and t5 cells transduced with shGFP or shDtymk-1 and then cultured with or without additional 150 μM dTTP in medium for 3 days.

(C) QPCR and Western blot analyses of Dtymk knockdown in the cells remaining in (B).

FIG. 5. Characterizations of Lkb1-wt and Lkb1-null cell lines

(A) Western blot analyses of the indicated protein expression in Lkb1-null and Lkb1-wt cells after shGFP, shDtymk-1, and shChek1-4 knockdown. Some Western blot bands were quantified by ImageJ, quantification values as indicated.

(B) Lkb1-wt and Lkb1-null cells in log-phase growth were fixed with cold 70% ethanol, stained with PI, and then analyzed with flow cytometry. 20,000 cells per line were analyzed.

(C) Lkb1-wt and Lkb1-null cells were plated into multiple chamber slides for overnight and then fixed for indirect immunofluorescence staining with anti-RPA32. The cells were observed with fluorescence microscopy. The data represent mean±SD for 200˜400 cells. A set of representative RPA32 images in the indicated cell lines are shown (D).

(E) Lkb1-wt and Lkb1-null cells in 6-well plates were transduced with shDtymk-1 or shGFP. Two sets of the cells were plated into multiple chamber slides: one was 2 days and the other was 3 days post transduction. After overnight culturing, the cells were labeled with 100 μM IdU for 20 min then fixed for indirect immunofluorescence staining with anti-BrdU. The data represent mean±SD for 200˜300 cells. Representative merged images of BrdU (red) and DAPI (blue) in the indicated cells are shown (F).

FIG. 6. CHEK1 inhibitors preferentially inhibit Lkb1/LKB1-null cell growth

(A) Survival graphs of drug-treated cells normalized to the survival of untreated cells. Lkb1-wt (634, 855, and 857), Lkb1-null (t2, t4, and t5), Human NSCLC LKB1-wt (H1792, Calu-1, and H358), and NSCLC LKB1-deficient (H23, H2122, and A549) cell lines were cultured and then plated into 96-well plates at 2000 cells/well in 100 μl medium containing the indicated concentrations of AZD7762 or CHIR124 for 3 days. Viable cells were then counted with Dojino's Cell Counting Kit-8 assay. The percentage of surviving cells under each drug treatment versus the concentration of drug was plotted as an inhibition curve. The data represent mean±SD for 3 repeats.

(B) Western blot analysis of γH2AX. The cell lines used in (A) were treated with AZD7762 or CHIR124 for 3 h and then lysed for Western blot analysis with the indicated antibodies.

(C) FACS analyses of γH2AX. Lkb1-wt (634, 855, and 857) and Lkb1-null (t2, t4, and t5) cells in log-phase growth were treated with 300 nM AZD7762 for 3 h, followed by flow cytometric analysis as described. 20,000 cells per treatment were analyzed.

FIG. 7. In vivo treatment

(A) Waterfall plot showing tumor response after two treatments of AZD7762. Each column represents one individual tumor, with data expressed relative to the pre-treatment tumor volume. Representative 18-FDG PET-CT images of mice from 3 different genotypes at baseline (left) and two days after initiation of treatment (right). The images shown were trans-axial slices containing the FDG-avid tumors, with CT providing anatomic references and PET showing the location and intensity of high tumor glucose utilization, where the SUVₘₐₓ was also recorded (e.g., SUV=3.2, and etc.).

(B) 1×10⁶ Lkb1-null (t2, t4, and t5) and human LKB1-deficient NSCLC (A549 and H2122) cells were implanted into athymic nude mice. When tumors grew to a diameter of 5 mm, the mice were intraperitoneally administered with AZD7762 daily at 25 mg/kg and/or Gemcitabine every 3 days at 50 mg/kg for 2 weeks. The data represent mean±SD for 2 mice. Lkb1-null and human LKB1-deficient NSCLC tumors treated with the indicated drug are shown. Quantification of tumor volume (mm³) are shown in (C).

FIG. 8. Proposed model for synthetic lethality relationships between LKB1 and DTYMK or CHEK1

Reduction in nucleotide pools and DTYMK expression in Lkb1-null cells leads to dUTP incorporation and replication stress (↓). Equivalent depletion of DTYMK reduces DTYMK activity and the dTTP pool below a critical threshold, which exacerbates this nucleotide stress (X) in Lkb1-null more than in Lkb1-wt cells. Similarly, upon depletion of CHEK1, cells enter mitosis before repairing their DNA, which exacerbates this nucleotide stress (X) in Lkb1-null more than in Lkb1-wt cells. Thus in Lkb1-null cells, both DTYMK and CHEK1 are more selectively required for resolution of replication stress.

FIG. 9. Scheme for creation of GEMM-derived cell lines

(A) GEMMs with genotypes Kras^{+/LSL-G12D}Tp53^{L/L}, and Kras^{+/LSL-G12D}Tp53^{L/L}Lkb1^{L/L} were treated with Adeno-Cre nasally at 6 weeks of age. After lung tumors developed, the tumor nodules were dissected, minced into small pieces, and plated in 100-mm cell culture dishes. Cells were passaged at least 5 times before their use in shRNA screening, compound screening, and metabolite profiling.

(B) The genetic constitution of the GEMM-derived cell lines with the indicated genotype was confirmed by PCR using water and genomic DNA from a Kras_{+/LSL-}^{G12D}Tp53^{L/L}Lkb1^{L/L} mouse tail as controls. Genotype, primer set, and primer sequence are listed.

FIG. 10. Growth curve analysis of Lkb1-wt and Lkb1-null cells.

Lkb1-wt (634, 855, and 857) and Lkb1-null (t2, t4, and t5) cells were plated into 96-well plates at 2000 cells/well in 100 μl medium. Viable cells were measured every 12 hours using Promega's CellTiter-Glo Assay. The data represent mean±SD for 4 replicates. Double time (hour) was calculated as [Duration of culture (hour)/log 2(Readout2/Readout1)].

FIG. 11. Efficacy of the shDtymks in knocking down Dtymk

(A) QPCR analysis of Dtymk and Chek1 knockdown in Lkb1-wt 634 cells. 634 cells were transduced with the indicated shDtymk or shChek1 lentiviruses for 3 days and then lysed for RNA extraction and RT-qPCR analysis. Relative gene expression is normalized to the cells transduced with shGFP. The data represent mean±SD for 3 replicates.

(B) Western blot analyses of expression levels of DTYMK, CHEK1 and γH2AX in the cells line used in FIG. 3B using β-actin as loading control. The cell lysates were collected at 2 days post-transduction (0 day post puro-selection in FIG. 3B).

FIG. 12. FACS analysis

Lkb1-null t4 cells were transduced with pCDH-Dtymk(R) or pCDH-Chek1(R) for 3 days, collected by trypsinization, and then submitted to sorting for GFP positive by live fluorescence-activated cell sorting (FACS). GFP-positive t4/Dtymk(R) and GFP-positive t4/Chek1(R) cells were collected, cultured, and then sorted for another two times. Arrowhead indicates the percentage of GFP-positive t4/Dtymk(R) (A) and GFP-positive t4/Chek1(R) (B) cells over the population.

FIG. 13. DTYMK and CHEK1 Expression

Western blot analysis of expression level of DTYMK and CHEK1 in the cells lines used in FIG. 3E using β actin as loading control. The cell lysates were collected at 2 days post-transduction (0 day post puro-selection in FIG. 3D).

FIG. 14. Efficacy of the shDTYMKs in knocking down human DTYMK

(A) Three human shDTYMKs (shDTYMK-D3, shDTYMK-D8, and shDTYMK-D10) and two mouse shDtymks (shDtymk-1 and shDtymk-3) were transduced into the human LKB1-wt NSCLC Calu-1 cells. Efficiency of knockdown of human DTYMK was determined by qPCR and Western blot.

(B) Western blot analysis of expression levels of DTYMK in the cell lines used in FIG. 4A using β actin as loading control.

FIG. 15. dTTP incorporation

Lkb1-wt and Lkb1-null cells were plated into 96 well plates with 4000 cells/well in 100 μL medium for overnight culturing then incubated with 0.25 μCi ³H-dTTP (Perkin Elmer, NET221H250UC) for 6 h and used 0.25 μCi ³H-deoxythymidine (Perkin Elmer, NET221H250UC) as positive and 0.25 μCi³H-dTTP/non-cells (medium alone) as negative controls. Cells were washed with PBS, trypsinized, and DNA was captured with a cell harvester on glass fiber filters Filtermat A (Perkin Elmer, #1450-421), which was then placed into a liquid scintillation counting container for counting on a scintillation beta-counter. The data represent mean±SD for 6 replicates.

### DETAILED DESCRIPTION OF THE INVENTION

The invention is based in part upon the surprising discovery that suppression of deoxythymidylate kinase (DTYMK) or checkpoint kinase 1 (CHEK1) is synthetically lethal with Lkb1-null status in lung cancer cells.

LKB1 is frequently mutated and inactivated in several common adult malignancies, including those arising in the lung, skin, and gastrointestinal and reproductive tracts. LKB1 mutations typically occur in conjunction with other oncogenic mutations, including activating KRAS mutation, and LKB1 loss significantly accelerates KRAS-driven lung tumorigenesis in mouse models. Currently there is no therapeutic approach to the treatment of LKB1 mutant cancers. High-throughput RNAi screens were performed to identify potential therapeutic targets for cancers harboring Lkb1 deletion mutations using cell lines derived from genetically engineered mice (GEM), and correlated the findings with those from kinase inhibitor and metabolite screens. These screens found suppression of either Dtymk or Chek1 to be synthetically lethal with Lkb1-null status in lung cancer cells. In addition, human non-small cell lung cancer cell lines that had LKB1 deletion mutations showed greater growth inhibition than controls in response to knockdown of DTYMK or CHEK1, and were also more sensitive to treatment with CHEK1 inhibitors. CHEK1 encodes checkpoint kinase 1, and its knockdown accumulates DNA damage. DTYMK encodes deoxythymidylate kinase (thymidylate kinase), and its knockdown inhibits dTTP biosynthesis and, consequently, DNA synthesis.

It is hypothesized that Lkb1 loss enhances dependence on these enzymes due to lower cellular levels of ATP and nucleotide metabolism, which makes these enzymes therapeutic targets in LKB1 mutant non-small cell lung cancer.

These results indicate that, therapy with DTYMK and/or CHEK1 inhibitor provides therapeutic benefits in Lkb1 mutant cancers such as lung cancer, skin cancer, gastrointestinal cancers and reproductive tract cancers.

Checkpoint Kinase 1

A checkpoint kinase 1 (CHEK1) inhibitor is a compound that decreases expression or activity of CHEK1. CHEK1 is an ATP-dependent serine-threonine kinase that phosphorylates Cdc25, an important phosphatase in cell cycle control, particularly for entry into mitosis.

A decrease in CHEK1 expression or activity is defined by a reduction of a biological function of the CHEK1. A biological function of CHEK1 includes phosphorylation of Cdc25, such as Cdc25A, Cdc25B, or Cdc25C, and initiation of phosphorylation signaling cascades that activate p53, inhibit Cdc2/cyclinB-mediated entry to mitosis, regulate the spindle checkpoint through AuroraB and BubR1, or initiate DNA repair processes through RAD51 and FANC proteins (i.e., FANCD2 or FANCE).

CHEK1 expression is measured by detecting a CHEK1 transcript or protein. CHEK1 inhibitors are known in the art or are identified using methods described herein. For example, a CHEK1 inhibitor is identified by detecting a premature or inappropriate checkpoint termination, phosphorylation status of downstream phosphorylation substrates (i.e. Cdc25A, Cdc25B, Cdc25C, Cdc2/cyclinB), efficiency of DNA repair, or imaging of spindles during mitosis.

The CHEK1 inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

The CHEK1 inhibitor is an antibody or fragment thereof specific to CHEK1.

Alternatively, the CHEK1 inhibitor is for example an antisense CHEK1 nucleic acid, a CHEK1-specific short-interfering RNA, or a CHEK1-specific ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense CHEK1 nucleic acid sequence, an anti-sense CHEK1 nucleic acid sequence or both. Optionally, the siRNA is constructed such that a single transcript has both the sense and complementary antisense sequences from the target gene, e.g., a hairpin.

Binding of the siRNA to a CHEK1 transcript in the target cell results in a reduction in CHEK1 production by the cell. The length of the oligonucleotide is at least 10 nucleotides and may be as long as the naturally-occurring CHEK1 transcript. Preferably, the oligonucleotide is 19-25 nucleotides in length. Most preferably, the oligonucleotide is less than 75, 50, 25 nucleotides in length.

The CHEK1 inhibitor is for example AZD7762 (CAS No. 860352-01-8), Go-6976 (CAS No. 136194-77-9), UCN-01 (CAS No. 112953-11-4), TCS2312 (CAS No. 838823-32-8), PD 407824 (CAS No. 622864-54-4), PF 477736 (CAS No. 952021-60-2), PD-321852, SB218078 (CAS No. 135897-06-2), LY2603618 (CAS No. 911222-45-2), LY2606368, CEP-3891, SAR-020106, debromohymenialdisine (CAS No. 75593-17-8), or CHIR124 (CAS No. 405168-58-3) mimetics or derivatives thereof. Other CHEK1 inhibitors are known in the art such as those described in Prudhomme, M. (2006) Recent Patents on Anti-Cancer Drug Discovery; 55-68, the contents of which is hereby incorporated by reference in its entirety.

Deoxythymidylate Kinase Inhibitors

A deoxythymidylate kinase (DTYMK) inhibitor is a compound that decreases expression or activity of DTYMK. DTYMK is a thymidylate kinase that is involved in cell cycle progression and cell growth stages

A decrease in DTYMK expression or activity is defined by a reduction of a biological function of the DTYMK. A biological function of DTYMK includes the catalysis of the phosphorylation of thymidine 5′-monophosphate (dTMP) to form thymidine 5′-diphosphate (dTDP) in the presence of ATP and magnesium. This process is essential for cell replication and proliferation. A decrease in DTYMK expression or activity can therefore be assessed by measuring the levels of thymidine 5′diphosphate (dTDP) or cell proliferation.

DTYMK expression is measured by detecting a DTYMK transcript or protein. DTYMK inhibitors are known in the art or are identified using methods described herein. For example, a DTYMK inhibitor is identified by detecting a decrease in thymidine 5′-diphosphate (dTDP) in the presence of ATP and magnesium.

The DTYMK inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

The DTYMK inhibitor is for example, a nucleoside analog (preferably a deoxythymidine analog), 5′trifluoromethyl-2′deoxyuridine (CAS No. 70-00-8), AZTMP (azidothymidine monophosphate) (CAS No. 29706-85-2) or derivatives thereof.

The DTYMK inhibitor is an antibody or fragment thereof specific for DTYMK.

Alternatively, the DTYMK inhibitor is for example an antisense DTYMK nucleic acid, a DTYMK-specific short-interfering RNA, or a DTYMK-specific ribozyme. By the term “siRNA” is meant a double stranded RNA molecule which prevents translation of a target mRNA. Standard techniques of introducing siRNA into a cell are used, including those in which DNA is a template from which a siRNA is transcribed. The siRNA includes a sense DTYMK nucleic acid sequence, an anti-sense DTYMK nucleic acid sequence or both. Optionally, the siRNA is constructed such that a single transcript has both the sense and complementary antisense sequences from the target gene, e.g., a hairpin.

Binding of the siRNA to a DTYMK transcript in the target cell results in a reduction in DTYMK production by the cell. The length of the oligonucleotide is at least 10 nucleotides and may be as long as the naturally-occurring DTYMK transcript. Preferably, the oligonucleotide is 19-25 nucleotides in length. Most preferably, the oligonucleotide is less than 75, 50, 25 nucleotides in length.

Therapeutic Methods

The growth of cells is inhibited, e.g. reduced, by contacting a Lkb1 null cell with a composition containing a compound that decreases the expression or activity of DTYMK and/or CHEK1. By inhibition of cell growth is meant the cell proliferates at a lower rate or has decreased viability compared to a cell not exposed to the composition. Cell growth is measured by methods know in the art such as, the MTT cell proliferation assay, cell counting, or measurement of total GFP from GFP expressing cell lines.

Cells are directly contacted with the compound. Alternatively, the compound is administered systemically.

The cell is a tumor cell such as a lung cancer, melanoma, a gastrointestinal cancer or a reproductive tract cancer or any other cancer harboring a LKB1 mutation. Gastrointestinal cancers include for example esophageal cancer, stomach cancer, gall bladder cancer, liver cancer, or pancreatic cancer. Reproductive tract cancers include for example, breast cancer, cervical cancer, uterine cancer, endometrial cancer, ovarian cancer, prostate cancer or testicular cancer.

In various aspects the cell has a Lkb1/LKB1 mutation, either in the gene or polypeptide. LKB1 activating mutations or Lkb1/LKB1 null mutations can be identified by methods known in the art. The mutation may be in the nucleic acid sequence encoding LKB1 polypeptide or in the LKB1 polypeptide, or both.

The methods are useful to alleviate the symptoms of a variety of cancers. Any cancer containing Lkb1/LKB1 mutation is amenable to treatment by the methods of the invention. In some aspects the subject is suffering from lung cancer, melanoma, a gastrointestinal cancer or a reproductive tract cancer.

Treatment is efficacious if the treatment leads to clinical benefit such as, a decrease in size, prevalence, or metastatic potential of the tumor in the subject. When treatment is applied prophylactically, “efficacious” means that the treatment retards or prevents tumors from forming or prevents or alleviates a symptom of clinical symptom of the tumor. Efficaciousness is determined in association with any known method for diagnosing or treating the particular tumor type.

Therapeutic Administration

The invention includes administering to a subject composition comprising a DTYMK and or a CHEK1 inhibitor.

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-proliferative agents or therapeutic agents for treating, preventing or alleviating a symptom of a cancer. A therapeutic regimen is carried out by identifying a mammal, e.g., a human patient suffering from a cancer that has a LKB1 mutation using standard methods.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The inhibitors are optionally formulated as a component of a cocktail of therapeutic drugs to treat cancers. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the therapeutic compounds are formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, conventional filler, and a tableting agent. Other formulations include an ointment, suppository, paste, spray, patch, cream, gel, resorbable sponge, or foam. Such formulations are produced using methods well known in the art.

Therapeutic compounds are effective upon direct contact of the compound with the affected tissue. Accordingly, the compound is administered topically. Alternatively, the therapeutic compounds are administered systemically. For example, the compounds are administered by inhalation. The compounds are delivered in the form of an aerosol spray from pressured container or dispenser which contains a suitable propellant, e.g., a gas such as carbon dioxide, or a nebulizer.

Additionally, compounds are administered by implanting (either directly into an organ or subcutaneously) a solid or resorbable matrix which slowly releases the compound into adjacent and surrounding tissues of the subject.

Screening Assays

The invention also provides a method of screening for therapeutic targets for treating cancers. In particular, the invention provides a method for identifying therapeutic targets for treating cancer by providing a cell that is null for an Lkb1 gene, an ATM gene, a TSC1 gene, a PTEN gene or a Notch gene and contacting the cell with a library of RNAi. Potential therapeutic targets are identified by determining what RNAi is lethal to the cell, decreases cell viability or inhibits cell growth. Assays for identification of potential therapeutic targets are known in the art, for example, MTT proliferation assay, cell growth curves, and analysis by staining and flow cytometry.

Cell Lines

The invention also provides a cell or a cell line for screening for therapeutic targets for treating cancer. In particular, the invention provides a cell expressing KRAS G12D and further comprising a disruption of the Trp53 gene, the Lkb1 gene or both, wherein the disruption results in decreased expression or activity of the Trp53 gene, the Lkb1 gene or both genes in the cell. In some embodiments, the cell is a lung cell, a melanoma cell, a pancreatic cell, an endometrial cell or an ovarian cell. In some embodiments, the cell is a cancer cell, for example a lung cancer cell, a melanoma cancer cell, a pancreatic cancer cell, an endometrial cancer cell or an ovarian cancer cell.

The cells can be generated using standard methods known in the art. For example, the the cells can be generated, isolated, and expanded from a genetically engineered mouse model (GEMM), as described herein using standard methods known in the art. For example, a GEMM harboring a conditional LSL-G12D Kras allele (Kras^{+/LSL-G12D}), a conditional Trp53-deficient allele (Trp53^{L/L}), and with or without a conditional Lkb1-deficient allele (Lkb1^{L/L}) can be generated by breeding (as described in Ji et al., 2007). The resulting Kras_{+/LSL-}^{G12D}Trp53^{L/L} and Kras^{+/LSL-G12D}Trp53^{L/L}Lkb1^{L/L} mice can be treated with Adenovirus-Cre through inhalation to cause recombination, to induce activation of Kras-G12D (Kras^{+/G12D}) and deletion of p53 (Trp^{53del/del}) and Lkb1 (Lkb1^{del/del}). Kras-G12D expression and deletion of p53 and Lkb1 can be detected by various standard methods known in the art, such as PCR genotyping and Western blot analysis. The cells can be harvested from the mice, such as cancer cells from a tumor sample from various tissues, such as the lung, skin, pancreas, uterus, or ovary.

Other methods of generating cells expresses KRAS G12D and further comprises a disruption of the Trp53 gene, the Lkb1 gene or both include introducing nucleic acid expression vectors comprising the KRAS G12D mutant gene and short hairpin sequences that target Trp53, Lkb1, or both into established cell lines via electroporation, transfection or viral infection. Alternatively, short hairpin sequences targeting Trp53, Lkb1 or both can be introduced to cells that already express KRAS G12D, G12E or another activating KRAS mutation known in the art. One ordinarily skilled in the art could produce stable cell lines after introduction of the gene and/or short hairpin(s) using standard methods known in the art. For example, short hairpin sequences targeting Trp53 or Lkb1 can be cloned into a lentiviral nucleic acid expression vector and viral particles can be generated. The target cells are transduced with the lentivirus and those that express the lentiviral constructs and hairpins at the desired levels can be selectively expanded using standard methods in the art.

### DEFINITIONS

As used herein, the term “null” refers to the presence, expression or activity status of a particular gene or genes. For example, an Lkb1 null cancer refer to those cancers that display a disruption in the Lkb1 gene, such that the levels of the Lkb1 gene, mRNA or protein or LKB1 protein activity is decreased. In some embodiments, the disruption in the gene can be caused by a mutation. Disruption of the gene can be detected by sequencing or genotyping methods known in the art. Detection of decreased mRNA or protein levels and protein activity can be detected by standard methods known in the art, for example qRT-PCR, microarray, immunoassays, Western blots or various activity assays.

The term “polypeptide” refers, in one embodiment, to a protein or, in another embodiment, to protein fragment or fragments or, in another embodiment, a string of amino acids. In one embodiment, reference to “peptide” or “polypeptide” when in reference to any polypeptide of this invention, is meant to include native peptides (either degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminal, C terminal or peptide bond modification, including, but not limited to, backbone modifications, and residue modification, each of which represents an additional embodiment of the invention. Methods for preparing peptidomimetic compounds are well known in the art and are specified, for example, in Quantitative Drug Design, C. A. Ramsden Gd., Chapter 17.2, F. Choplin Pergamon Press (1992).

As used interchangeably herein, the terms “oligonucleotides”, “polynucleotides”, and “nucleic acids” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, all as described herein.

The term “homology”, when in reference to any nucleic acid sequence indicates a percentage of nucleotides in a candidate sequence that are identical with the nucleotides of a corresponding native nucleic acid sequence. Homology may be determined by computer algorithm for sequence alignment, by methods well described in the art. For example, computer algorithm analysis of nucleic acid or amino acid sequence homology may include the utilization of any number of software packages available, such as, for example, the BLAST, DOMAIN, BEAUTY (BLAST Enhanced Alignment Utility), GENPEPT and TREMBL packages.

As used herein, the term “substantial sequence identity” or “substantial homology” is used to indicate that a sequence exhibits substantial structural or functional equivalence with another sequence. Any structural or functional differences between sequences having substantial sequence identity or substantial homology will be de minimus; that is, they will not affect the ability of the sequence to function as indicated in the desired application. Differences may be due to inherent variations in codon usage among different species, for example. Structural differences are considered de minimus if there is a significant amount of sequence overlap or similarity between two or more different sequences or if the different sequences exhibit similar physical characteristics even if the sequences differ in length or structure. Such characteristics include, for example, the ability to hybridize under defined conditions, or in the case of proteins, immunological crossreactivity, similar enzymatic activity, etc. The skilled practitioner can readily determine each of these characteristics by art known methods.

Additionally, two nucleotide sequences are “substantially complementary” if the sequences have at least about 70 percent or greater, more preferably 80 percent or greater, even more preferably about 90 percent or greater, and most preferably about 95 percent or greater sequence similarity between them. Two amino acid sequences are substantially homologous if they have at least 50%, preferably at least 70%, more preferably at least 80%, even more preferably at least 90%, and most preferably at least 95% similarity between the active, or functionally relevant, portions of the polypeptides.

To determine the percent identity of two sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, at least 30%, 40%, 50%, 60%, 70%, 80%, or 90% or more of the length of a reference sequence is aligned for comparison purposes. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

Thus, treating may include suppressing, inhibiting, preventing, treating, or a combination thereof. Treating refers inter alia to increasing time to sustained progression, expediting remission, inducing remission, augmenting remission, speeding recovery, increasing efficacy of or decreasing resistance to alternative therapeutics, or a combination thereof. “Suppressing” or “inhibiting”, refers inter alia to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. The symptoms are primary, while in another embodiment, symptoms are secondary. “Primary” refers to a symptom that is a direct result of the proliferative disorder, while, secondary refers to a symptom that is derived from or consequent to a primary cause. Symptoms may be any manifestation of a disease or pathological condition.

The “treatment of cancer or tumor cells”, refers to an amount of peptide or nucleic acid, described throughout the specification, capable of invoking one or more of the following effects: (1) inhibition of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, “an ameliorated symptom” or “treated symptom” refers to a symptom which approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer to shrink rr or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head and neck, kidney, lung, non-small cell lung, melanoma, mesothelioma, ovary, sarcoma, stomach, uterus and Medulloblastoma. Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

A “proliferative disorder” is a disease or condition caused by cells which grow more quickly than normal cells, i.e., tumor cells. Proliferative disorders include benign tumors and malignant tumors. When classified by structure of the tumor, proliferative disorders include solid tumors and hematopoietic tumors.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

By the term “modulate,” it is meant that any of the mentioned activities, are, e.g., increased, enhanced, increased, augmented, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values.

As used herein, the term “administering to a cell” (e.g., an expression vector, nucleic acid, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

As used herein, “molecule” is used generically to encompass any vector, antibody, protein, drug and the like which are used in therapy and can be detected in a patient by the methods of the invention. For example, multiple different types of nucleic acid delivery vectors encoding different types of genes which may act together to promote a therapeutic effect, or to increase the efficacy or selectivity of gene transfer and/or gene expression in a cell. The nucleic acid delivery vector may be provided as naked nucleic acids or in a delivery vehicle associated with one or more molecules for facilitating entry of a nucleic acid into a cell. Suitable delivery vehicles include, but are not limited to: liposomal formulations, polypeptides; polysaccharides; lipopolysaccharides, viral formulations (e.g., including viruses, viral particles, artificial viral envelopes and the like), cell delivery vehicles, and the like.

### EXAMPLES

### Example 1

### General Methods

Generation of GEMM Cell Lines

Generation of GEMM-derived cell lines. Genetically engineered mouse model (GEMM) harboring a conditional LSL-G12D Kras allele (Kras^{+/LSL-G12D}), a conditional Trp53-deficient allele (Trp53^{L/L}), and with or without a conditional Lkb1-deficient allele (Lkb1^{L/L}) were generated by breeding (Ji et al., 2007). At the age of 6 weeks, the Kras_{+/LSL-}^{G12D} Trp53^{L/L} and Kras^{+/LSL-G12D}Trp53^{L/L}Lkb1^{L/L} mice were treated with Adenovirus-Cre through inhalation to cause recombination, leading to activation of Kras-G12D (Kras^{+/G12D}) and deletion of p53 (Trp53^{del/del}) and Lkb1 (Lkb1^{del/del}) (Ji et al., 2007). Six to nine weeks after Adenovirus-Cre administration, the mice were sacrificed and lung tumor nodules were harvested, finely minced, and cultured in 100 mm dishes with RPMI 1640/10% FBS/1% pen-strep/2 mM L-Glutamine. After 5 passages, frozen stocks of these short-term cultures were prepared, and the lines characterized by genotyping and Western blot analysis.

293T, NCI-H1792, Calu-1, H358, H23, H2122, and A549 were obtained from the American Type Culture Collection. 293T was grown in DMEM/10% FBS/1% pen/strep/2 mM L-Glutamine, and the remaining lines were grown in RPMI 1640/10% FBS/1% pen-strep/2 mM L-Glutamine. All cells were cultured at 37° C. in a humidified incubator with 5% CO₂.

Large-Scale Pooled shRNA Library Screening and Array-Based Validation

(1) Construction of Pooled Murine shRNA Library and Virus Pool Production

The murine 40K pool of 40,021 shRNA plasmids, covering 8391 genes, from The RNAi Consortium was assembled by combining 11 normalized sub-pools of ˜3600 shRNA plasmids each. Each sub-pool was used to transform ElectroMAX DH5α-E cells (Invitrogen) by electroporation and plated onto 5 24□24 cm² bioassay dishes (Nunc). DNA was purified from the plated transformants using a HiSpeed Plasmid Maxi Kit (Qiagen). These sub-pools were then combined to create the 40K shRNA pool. 2 μg of this pool was used to transform ElectroMax DH5α-E cells and plated onto 40 24×24 cm bioassay dishes. DNA was purified from the plated transformants and used for virus production. A complete list of shRNAs along with unique TRCN identifiers is publicly available (http://www.broadinstitute.org/rnai/public/).

Production of lentivirus from the murine 40K shRNA pool was performed as described previously (Luo et al., 2008). A single batch of ˜1.5 L of virus was aliquoted and frozen at −80° C. for all infections.

(2) Large-Scale Virus Infection and Cell Propagation

Infections were performed as described (Luo et al., 2008) with the following modifications. To determine viral volume that would produce a Multiplicity of Infection (MOI) of 0.2-0.5 for each cell line, cells were infected with a titration of 6 different volumes (0-400 μl) of virus and cultured in the presence or absence of puromycin. Each cell line was infected with the shRNA pool in triplicate as follows. 3.7×10⁷ of 634, 855, or 857 cells; 5.4×10⁷ of t5 cells; and 7.2×10⁷ of t4 or t5 cells were resuspended in 24 ml of medium containing 8 μg/ml polybrene and the appropriate volume of 40K library lentiviruses was added. This mixture was seeded into a 12-well plate at ˜2 ml per well. A spin infection was performed by centrifugation at 930×g for 2 h at 30° C. Immediately after spinning, supernatants were gently aspirated off and fresh medium was added to the 12-well plates. After 20 h the 12 wells from each replicate were trypsinized, cells combined, and plated in 3 T225-flasks containing 60 ml of medium containing puromycin. The cell were passaged every 2-3 days by trypsinizing all flasks of a replicate, combining the cells, then seeding 2 T225 flasks with a total of 1.1×10⁷ cells. The remaining cells were spun down and resuspended in 1 ml PBS and frozen at −20 degrees. This process was continued for at least 16 population doublings with the final collection frozen in 1 ml PBS at −20 degrees, as above. Puromycin selection was maintained for the entire experiment.

(3) Infection Calculation

20 h after large-scale infection, a small fraction of cells (1.5-3×10⁵) from each replicate were plated into each well of 6-well plates in the presence or absence of puromycin. Control wells with 100% uninfected cells were included to verify complete puromycin killing of uninfected cells. 96 h later, viable cells were counted. The infection rate was determined by the number of viable cells selected in puromycin divided by the number of viable cells without puromycin selection. Screening continued only when the infection rates were within the range of 20-50% in order to yield sufficient number cells to obtain an average infection rate of at least 200 cells/shRNA.

(4) Determination of shRNA Representation by Sequencing

Harvested cells were resuspended in 1 ml PBS, and genomic DNA was isolated using the QIAamp Blood Mini kit (Qiagen). For PCR amplification of shRNA sequences, a minimum of 50 μg of genomic DNA was used as template for each replicate. Therefore, multiple PCR reactions were performed, each using 3 μg of genomic DNA per 50 μl reaction volume. The hairpin region was PCR amplified from the purified genomic DNA using the following conditions: 5 μl primary PCR primer mix, 4 μl dNTP mix, 1×Ex Taq buffer, 0.75 μl of Ex TaqDNA polymerase (TaKaRa), and 6 μg genomic DNA in a total reaction volume of 50 pl. Thermal cycler PCR conditions consisted of heating samples to 95° C. for 5 min; 15 cycles of 94° C. for 30 sec, 65° C. for 30 sec, and 72° C. for 20 sec; and 72° C. for 5 min. PCR reactions were then pooled per sample. A secondary PCR step was performed containing 5 uM of common barcoded 3′ primer, 8 dNTP mix, 1×Ex Taq buffer, 1.5 μl Ex TaqDNA polymerase, and 30 μl of the primary PCR mix for a total volume of 90 μl. 10 μl of independent 5′ barcoded primers was then added into each reaction, after which the 100 μl total was is divided into two 50 μl final reactions. Thermal cycler conditions for secondary PCR were as follows: 95° C. for 5 min; 15 cycles of 94° C. for 30 sec, 58° C. for 30 sec, and 72° C. for 20 sec; and 72° C. for 5 min. Individual 50 μl reactions from the same 5′ barcoded primer were then re-pooled. Reactions were then run on a 2% agarose gel and intensity-normalized. Equal amounts of samples were then mixed and gel-purified using a 2% agarose gel. This master mix containing all individually barcoded samples was sequenced using a custom-sequencing primer on the Illumina HiSeq2000.

(5) Illumina Data Extraction and Normalization

Raw Illumina sequence reads were extracted for each shRNA in the murine 40k pool for each experimental sample. Raw reads were normalized across Illumina sequencing lanes by generating a value, shRNA reads/10⁶ total reads, by dividing the individual shRNA raw reads/the total reads for a sample×10⁶. This allowed comparison of data across several Illumina lanes, each with slightly different total raw reads.

For every shRNA a Log 2 Fold Change (Log 2FC) value was calculated from the difference in the abundance in the late time point sample and the initial sample (4 days post infection).

(6) Collapsing shRNA Scores to Gene Rankings

The GENE-E program (http://www.broadinstitute.org/cancer/software/GENE-E) (Luo et al., 2008) was used to collapse shRNA Log 2FC values to gene rankings by 3 complementary methods. These methods included 1) ranking genes by their highest shRNA Log 2FC score, 2) ranking genes based on the rank of the weighted second best score (ranked top shRNA25% weight+second best shRNA 75% weight) and 3) ranking genes using a KS statistic in a GSEA-like approach (RIGER) for scoring genes based on the p-value rank of the Normalized Enrichment Scores (NES) (Luo et al., 2008). The NES represents the bias of the set of shRNAs targeting each gene towards the phenotype of interest, for example depletion in one class of samples vs. a second class.

To assess the significance of a gene score obtained by the second best or KS scoring methods described, p-values were computed based on 10,000 random samplings of shRNAs to create artificial genes with the same number of shRNAs as the gene of interest (correcting for different set sizes of shRNA targeting different genes). The p-value reflects the number of times such an artificially constructed gene received a score as good as or better than the gene of interest. Therefore, the smaller the p-value the less likely such a gene score could have been obtained at random.

On average, 58% of the shRNA suppress their target genes greater than 70% using qPCR measurements of endogenous transcript levels (The RNAi Consortium, unpublished data). Thus a simple average of shRNA scores is not ideal since not all shRNAs are effective. Since the single shRNA and second best shRNA methods depend only on the 1 to 2 shRNAs of strongest effect, the influence of ineffective shRNAs on gene scores is minimized. The KS statistic however considers all shRNAs from each gene in producing a gene score. It is thus more sensitive to cases for example in which all five shRNAs score moderately for depletion. Since a higher false positive rate with the single shRNA ranking method is predicted due to off-target effects compared to the other methods, only the top 100 genes identified by this method were selected for further analysis, while the top 200 genes from each of the other two methods were selected. A union was taken of the genes identified by these three methods.

(7) Array-Based Viral Infection and Cell Proliferation Assay

For array-based viral infection and assessment of proliferation, 2.5 μl virus was mixed with 250 cells in 100 μl medium containing 8 μg/ml polybrene per well in 96-well plates. The plates were spun at 2250 rpm/37° C. for 30 min. Immediately after spinning, supernatants were gently aspirated off and 100 μl fresh medium was added to each well of the 96-well plates. After 2 days incubation, medium was gently aspirated off and 100 fresh medium containing 3 μg/ml puromycin was added to each well of the 96-well plates. The plates were back to culture for additional 3 days and then the viable cells were monitored by alamarBlue (Invitrogen) assay according to manufacturer's instructions.

High-Throughput Kinase Inhibitor Screening

Cells were cultured, collected by trypsinization, washed with media, and then resuspended at 7500 cells/ml. 50 μl of the cell suspension, containing 375 cells, was plated into each well of 384-well plates, followed by addition of 33 nl of the 1 mM library compound, covering 998 previously reported clinical and preclinical kinase inhibitors, by pin transfer to result in a final concentration of 660 nM in 0.066% DMSO. The cells were cultured for 2 days, and then viable cells were measured with CellTiter-Glo Luminescent Cell Viability Assay. All reactions were performed in duplicate plates.

Non-Targeted Flow-Injection-Analysis Mass Spectrometry for Metabolomics

Cells were plated into 6-well plates in RPMI 1640/10% dialyzed FBS/1% pen/strep and medium was changed daily. When the cells reached 80% confluence, they were washed 3 times with warm washing buffer (75 mM ammonium carbonate, pH7.4), and plates were then immediately placed on dry ice. 500 μl of extraction buffer (80% methanol, −80° C.) was added to each well and the plates were kept on dry ice for 15 min. The supernatants were collected into 1.5 ml eppendorf tubes, and another 500 μl of cold extraction buffer was added to each well. After 15 min incubation on dry ice, the supernatant and the cells were collected and pooled with the previously collected supernatant. The tubes were spun at 3750 rpm/4° C. for 30 min, and then supernatants were transferred into fresh tubes and saved at −80° C. All reactions were performed with 5 replicates.

Prior to mass spectrometer injection, dried extracts were reconstituted in LCMS grade water. Non-targeted, flow-injection time-of-flight mass spectrometry was performed as described (Fuhrer et al., 2011). Briefly, the mass spectrometry platform consists of an Agilent Series 1100 LC pump coupled to an Agilent 6520 Series Quadrupole Time-of-flight mass spectrometer (Agilent, Santa Clara, Calif.) equipped with an electrospray source operated in negative and positive mode. The flow rate was 150 μl/min of mobile phase consisting of isopropanol/water (60:40, v/v) buffered with 5 min ammonium carbonate at pH 8.5. Mass spectra were recorded from m/z 50 to 1000 with a frequency of 1.4 spectra/s for 0.48 min using the highest resolving power (4 GHz HiRes). All steps of data processing and analysis were performed with Matlab R2010b (The Mathworks, Natick) using functions native to the Bioinformatics, Statistics, Database, and Parallel Computing toolboxes.

Plasmid Constructs and Mutagenesis

All pLKO.1-shRNAs used in the current study were purchased from Broad Institute. Wild type cDNAs encoding murine DTYMK (BC030178) and CHEK1 (BC100386) were purchased from Thermo Scientific. ShRNA-resistant cDNAs were made by mutagenesis PCR and then subcloned into the BamH I and Not I sites of pCDH-CMV-MCS-EF1-Puro (pCDH) vector (System Biosciences) to generate pCDH-Dtymk(R) and pCDH-Chek1 (R), respectively. Silent mutation of Dtymk resistant to shDtymk-3 was introduced by primer pair (forward) 5′-GAGATTGGTAAACTCCTCAACTCGTATCTGGAAAAGAAAA-3′ (SEQ ID NO: 1) and (reverse) 5′-CAGATACGAGTTGAGGAGTTTACCAATCTCCGTTGATCTT-3′ (SEQ ID NO: 2); and silent mutation of Chek1 resistant to shChek1-4 was introduced by primer pair (forward) 5′-CAGTGGAAAAAAAGCTGCATGAATCAGGTT-3′ (SEQ ID NO: 3) and (reverse) 5′-ATGCAGCTTTTTTTCCACTGATAGCCCAAC-3′ (SEQ ID NO: 4). All mutagenized plasmids were confirmed by sequencing.

Lentiviral Production of Individual shRNAs and Target Cell Transduction

Lentiviral production and target cell transduction were performed according to previously description (Moffat et al., 2006). Briefly, 293T cells were co-transfected with pLenti-vector, pCMV-dR8.74psPAX2, and pMD2.G using TransIT-LT1 transfection reagent (Mirus). Thirty-six h after transfection, the supernatant was harvested and spun at 3000 rpm/4° C. for 10 min, and then incubated with target cells in the presence of 8 μg/ml polybrene (Sigma) for 24 h. Two days after infection, the cells were collected for further analysis as indicated in the presence of 3 μg/ml Puromycin (Invitrogen).

Cell Proliferation Assay

Cells were plated into 96-well plates at 2000 cells per well in 100 μl, with addition of puromycin at 3 μg/ml for shRNA lentivirus infected cells, or with addition of variable doses of drug for drug treatment effects. Viable cells were measured daily or for a period of up to 3 days either by CellTiter-Glo Luminescent Cell Viability Assay (Promega) or by Cell Counting Kit-8 (CCK-8) (Dojindo) according to the manufacturer's instructions. All proliferation assays were performed in triplicate wells.

RNA Extraction, Reverse Transcription, and RT-Quantitative PCR

Total RNAs of cultured cells were extracted using Trizol (Invitrogen). To generate cDNA, 1 μg total RNA was reverse transcribed (RT) using ImProm-II RT system (Promega) according to the manufacturer's instructions. Real-time quantitative PCR (qPCR) reaction was performed in a final volume of 20 μl containing 10 μl 2×SYBR Green PCR master mix (Applied Biosystems), 1 μl 10 jiM forward primer, 1 μl 10 μM reverse primer, and cDNA corresponding to 45 ng RNA using StepOnePlus Real-Time PCR System (Applied Biosystems) according to the manufacturer's protocol. All reactions were performed in triplicate wells. All qPCR primers were designed using Primer3. The primers were as follows, for Dtymk: (forward) 5′-GTGCTGGAGGGTGTGGAC-3′ (SEQ ID NO: 5), and (reverse) 5′-TTCAGAAGCTTGCCGATTTC-3′ (SEQ ID NO: 6); for Chek1: (forward) 5′-CTGGGATTTGGTGCAAACTT-3′ (SEQ ID NO: 7), and (reverse) 5′-GCCCGCTTCATGTCTACAAT-3′ (SEQ ID NO: 8); for mouse β-Actin: (forward) 5′-CTAAGGCCAACCGTGAAAAG-3′ (SEQ ID NO: 9), (reverse) 5′-GACCAGAGGCATACAGGGAC-3′ (SEQ ID NO: 10); and for human β-Actin: (forward) 5′-CAAGAGATGGCCAGGGCTGCT-3′ (SEQ ID NO: 11), and (reverse) 5′-TCCTTCTGCATCCTGTCGGCA-3′ (SEQ ID NO: 12). All qPCR reactions were performed in triplicate.

Western Blot and Antibodies

Upon reaching 80-90% confluence, cells in 6-well plates were lysed with 250 of 1×LDS Sample Buffer (Invitrogen) with a protease and phosphatase inhibitor cocktail (Thermo), sonicated, and then boiled for 5 min. Twenty microliters of each sample were resolved with SDS-PAGE, and the samples were analyzed by immunoblotting with the indicated antibodies. Protein was visualized with horseradish peroxidase-conjugated secondary antibodies (Amersham Biosciences) and an enhanced Chemiluminescent substrate kit (Thermo). Anti-DTYMK was from ProteinTech; anti-CHEK1, anti-γH2AX, and anti-RPA32 were from Cell Signaling; anti-phospho RPA32(S4/S8) was from Bethyl Laboratories, anti-RNR-R2 was from Santa Cruz; anti-BrdU was from BD Biosciences; and anti-β-actin was from Sigma.

Prepare FACS Samples

Upon reaching 80-90% confluence, cells were collected by trypsin and washed once with PBS. For immunofluorescence staining, 1×10⁶ fixation/permeabilization solution from the BD cytofix/cytoperm kit, incubated on ice for 45 min, and stained following the instructions provided with the kit. All FACS was performed at Dana-Farber Cancer Institute Flow Cytometry Core, and the data were analyzed using FlowJo.

In Vivo Imaging Studies

Each mouse was imaged using 18-FDG PET-CT before and after two treatments of AZD7762, as described. For each tumor, hypermetabolic activity was quantified using the maximum standard uptake value_{(SUVmax)} obtained from the FDG-PET imaging. The changes in hypermetabolic activity after treatment were normalized by their related baseline values and then were compared by tumor genotype. For xenograft study, 7 week old female athymic nude mice were used for cell line implantation and treatments as described in the text.

For FDG-PET imaging, each mouse was (1) placed on a special diet for approximately 16 hours designed to lower background blood glucose levels while reducing the stress associated with fasting; (2) injected with approximately 14 MBq@250 μl of ¹⁸F-FDG through catheterized tail vain administration after being warmed for at least an hour; (3) monitored for one hour to allow for ¹⁸F-FDG uptake; (4) anesthetized by inhalation of a mixture of sevoflurane and oxygen; (5) scanned with a low-dose CT acquisition protocol (50 kVp, 0.5 mA, 220 degree rotation, 600 ms/degree exposure time, 60 μm reconstruction pixel size), followed by a PET data acquisition protocol (350-650 key energy window, 10 minutes listmode acquisition, 3D rebinning followed by OSEM-MAP reconstruction) on a multi-modality preclinical imaging system (Inveon™, Siemens Healthcare). With the co-registered CT providing anatomic information, reconstructed FDG-PET images were analyzed using Inveon Research Workplace (Siemens Healthcare), where lung tumors were identified and quantified by SUVₘₐₓ.

### Example 2

### Generation of Lung Cancer Cell Lines from Gem Models

Although GEMMs (genetically engineered mouse models) have been widely used in tumorigenesis and treatment studies, their use in high-throughput analyses have been limited to date. In the current study, using genetically engineered Kras^{+/LSL-G12D}Trp53^{L/L} and Kras^{+/LSL-G12D}Trp53^{L/L}Lbk1^{L/L} mice, lung tumors were induced by intranasal administration of Adenovirus-Cre and established cell lines from tumor nodules (FIG. 9A). Each cell line was derived from a discrete lung tumor nodule, and the genotypes of each cell line were confirmed by PCR (FIG. 9B). Three different screens were conducted using 6 GEMM-derived cell lines. Three of these lines, named 634, 855, and 857, were derived from Kras^{+/LSL-G12D}Trp53^{L/L} mice, expressed Kras-G12D and had Trp53 deletion (referred to as Kras/p53 or Lkb1-wt). The other three lines, named t2, t4, and t5, were derived from Kras^{+/LSL-G12D}Trp53^{L/L}Lkb1^{L/L} mice, expressed Kras-G12D and had deletions of both Trp53 and Lkb1 (referred to as Kras/p53/Lkb1 or Lkb1-null).

### Example 3

### Identification of Selective Essential Genes in Kras/P53/Lkb1/GEMM-Derived Cell Lines

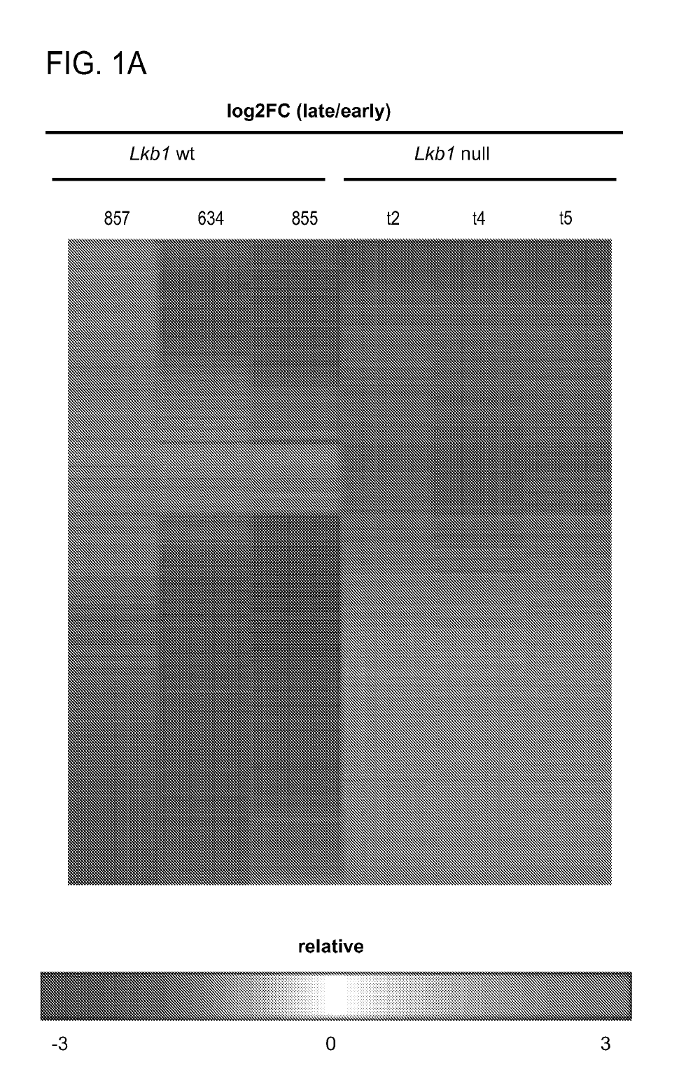

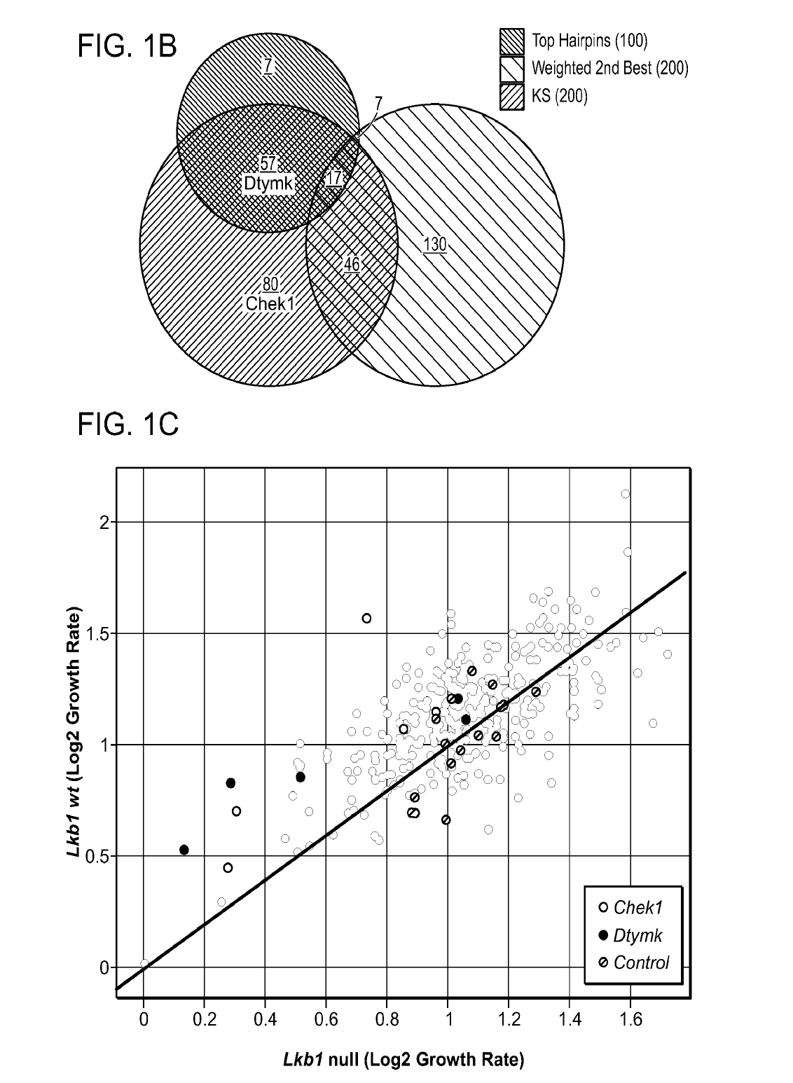

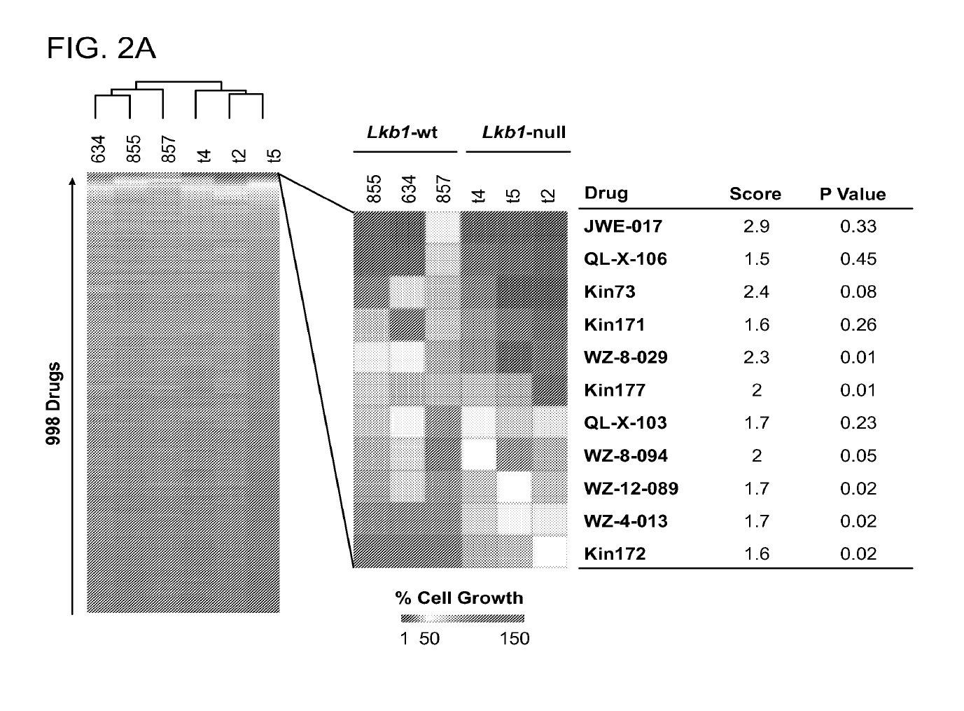

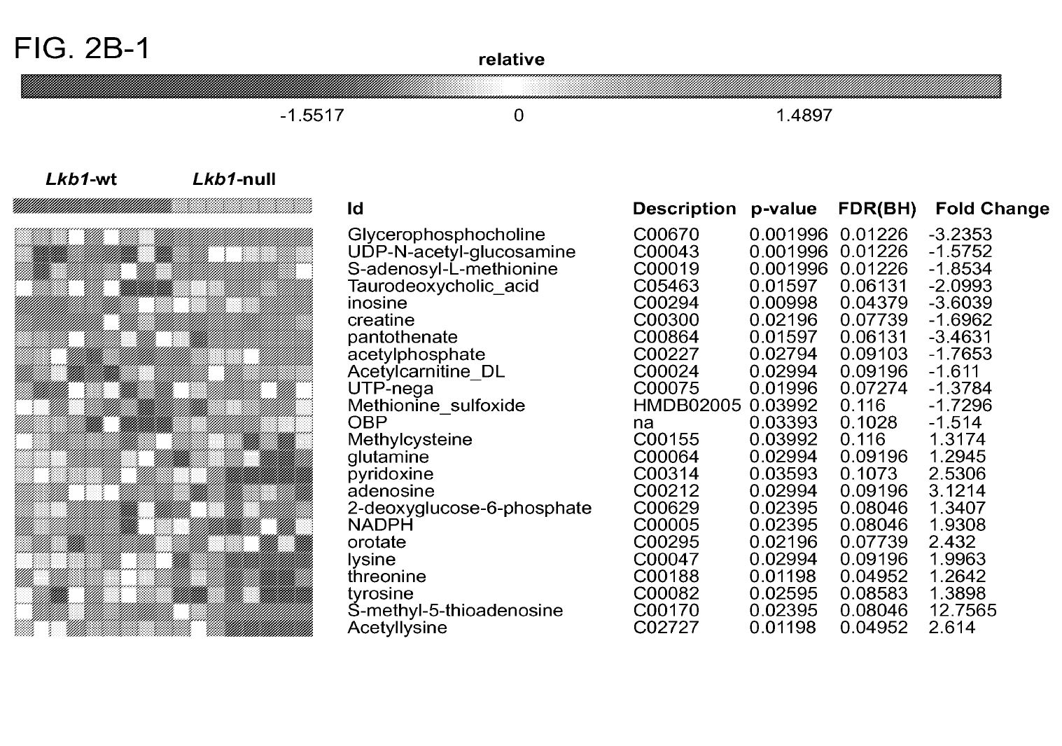

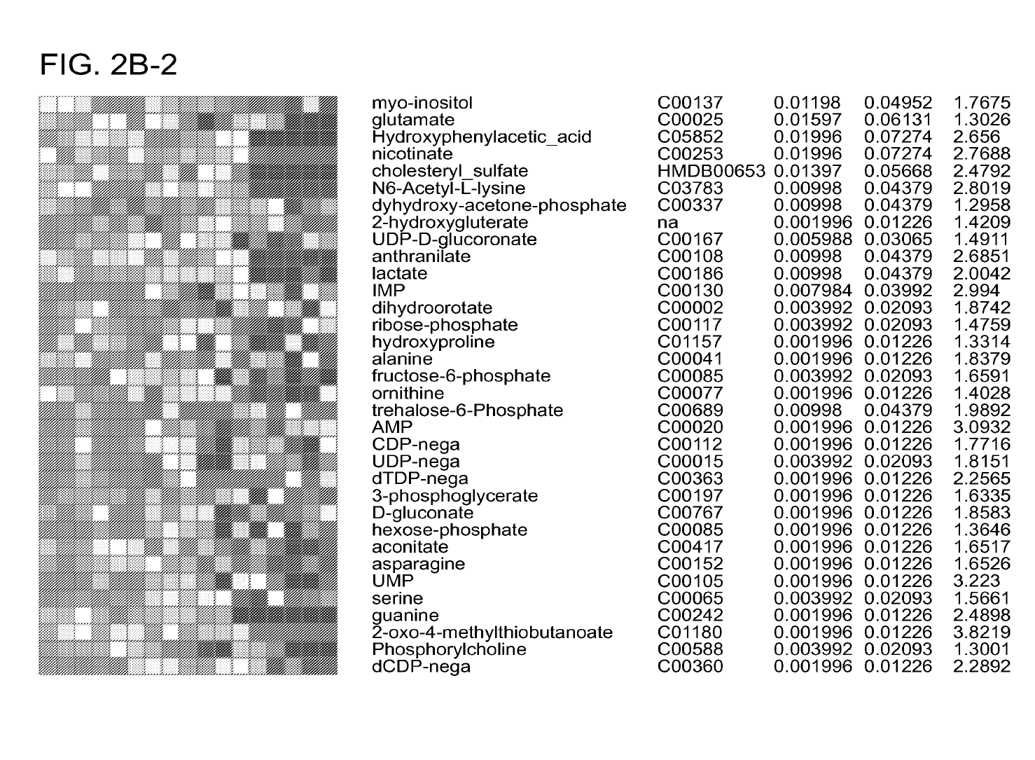

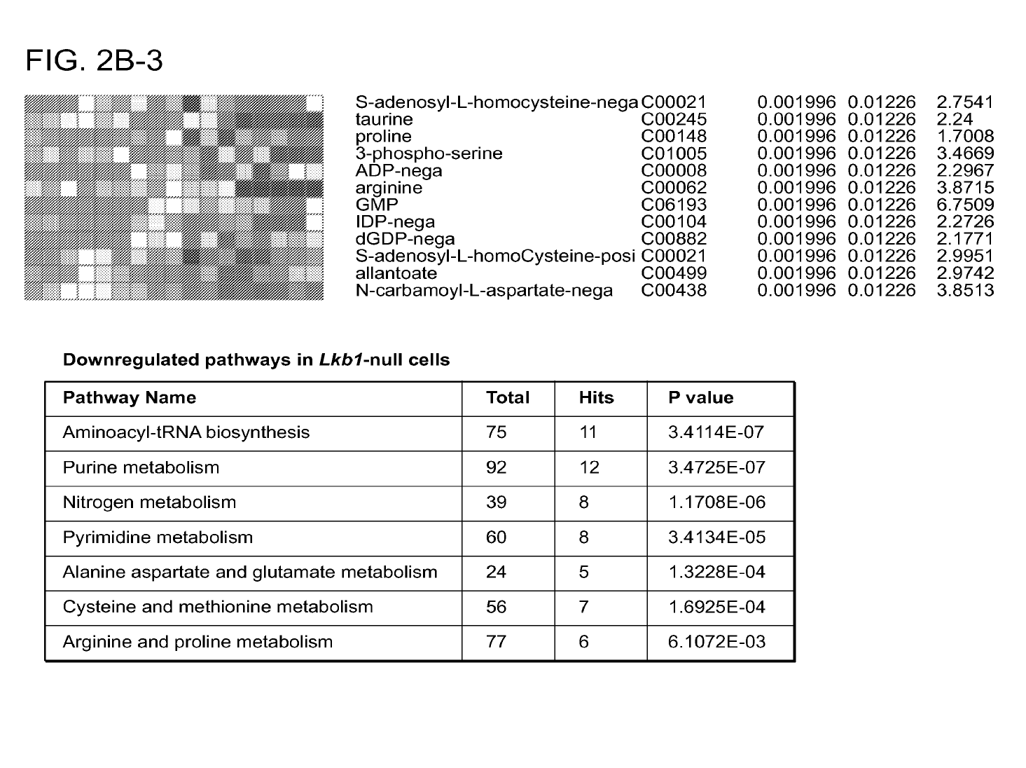

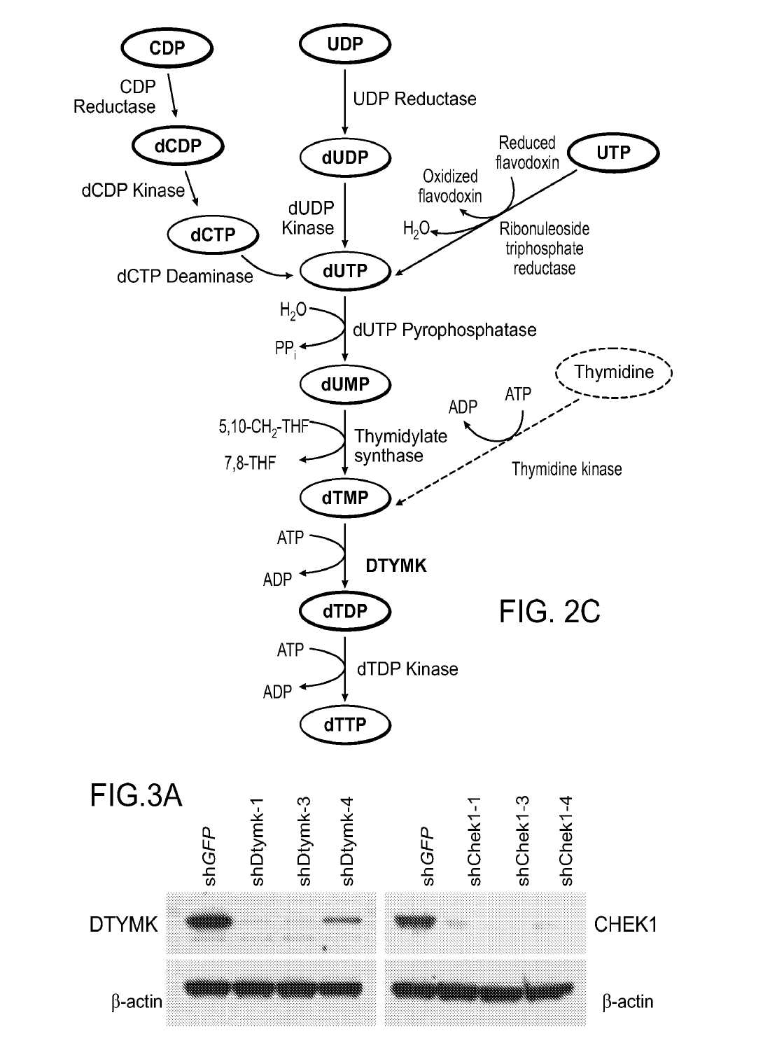

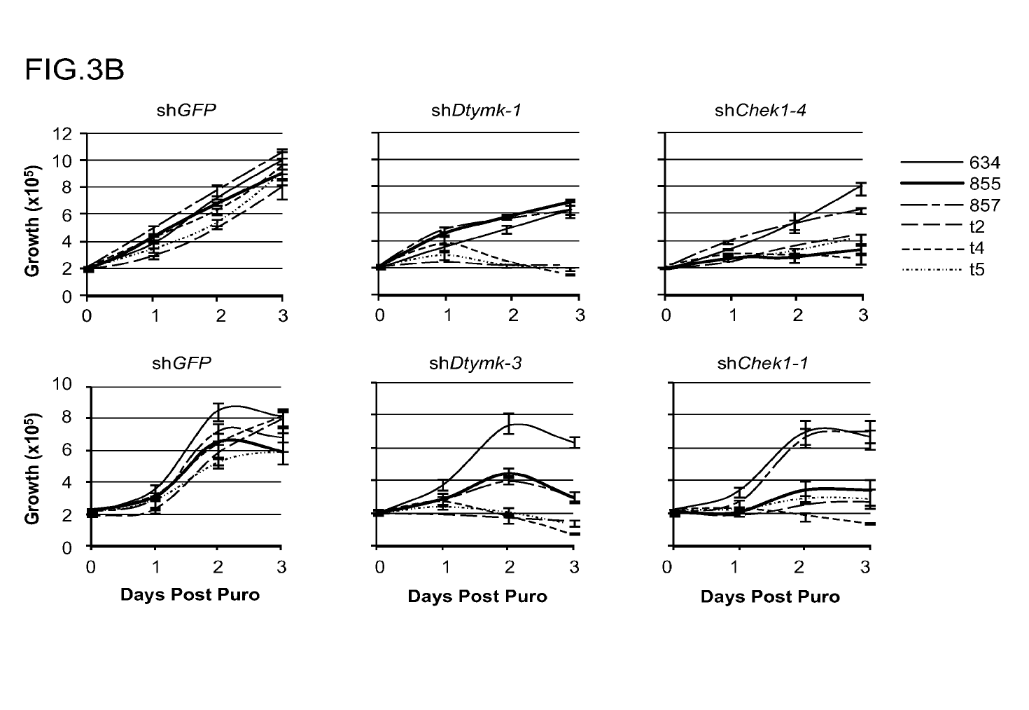

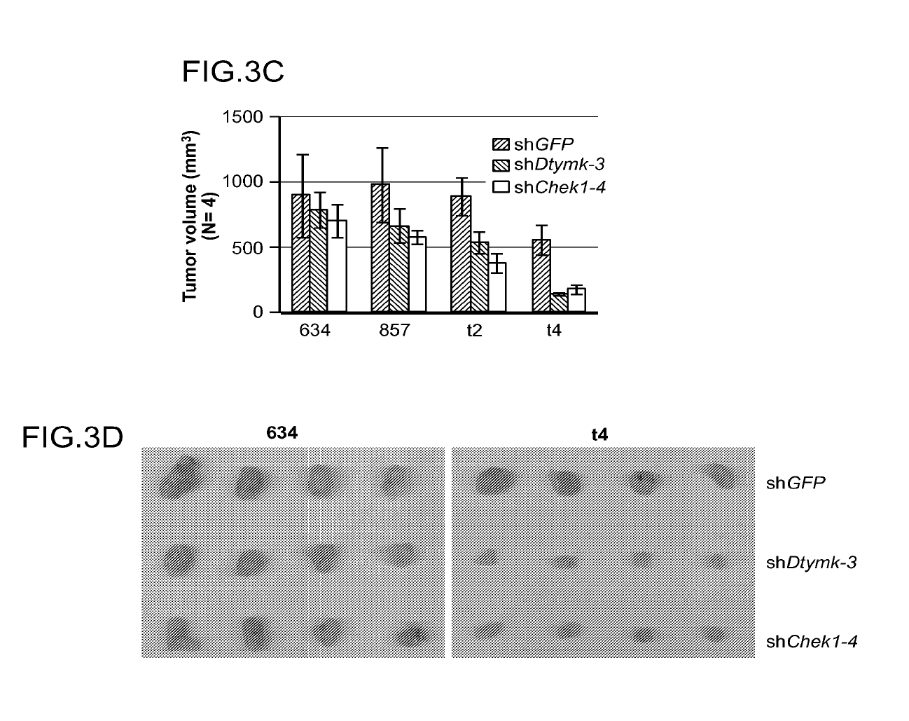

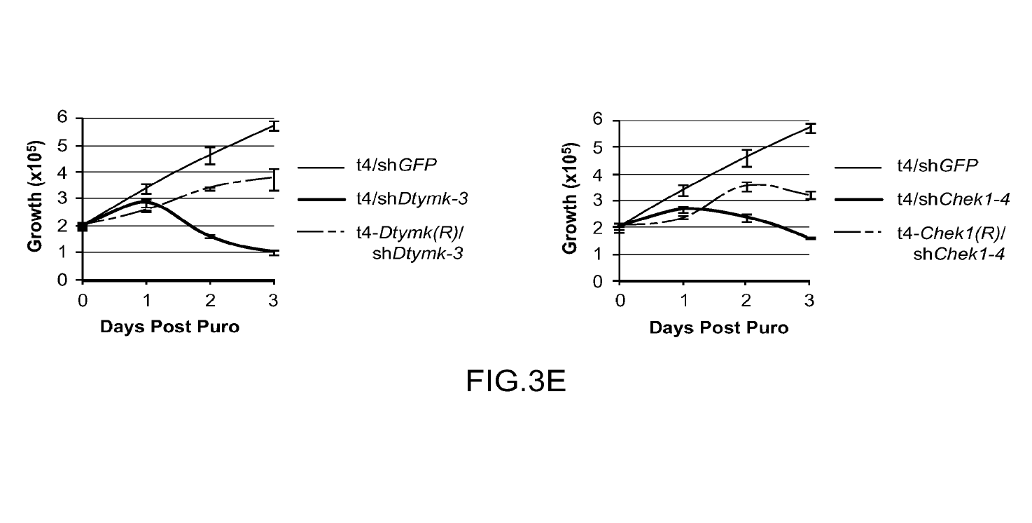

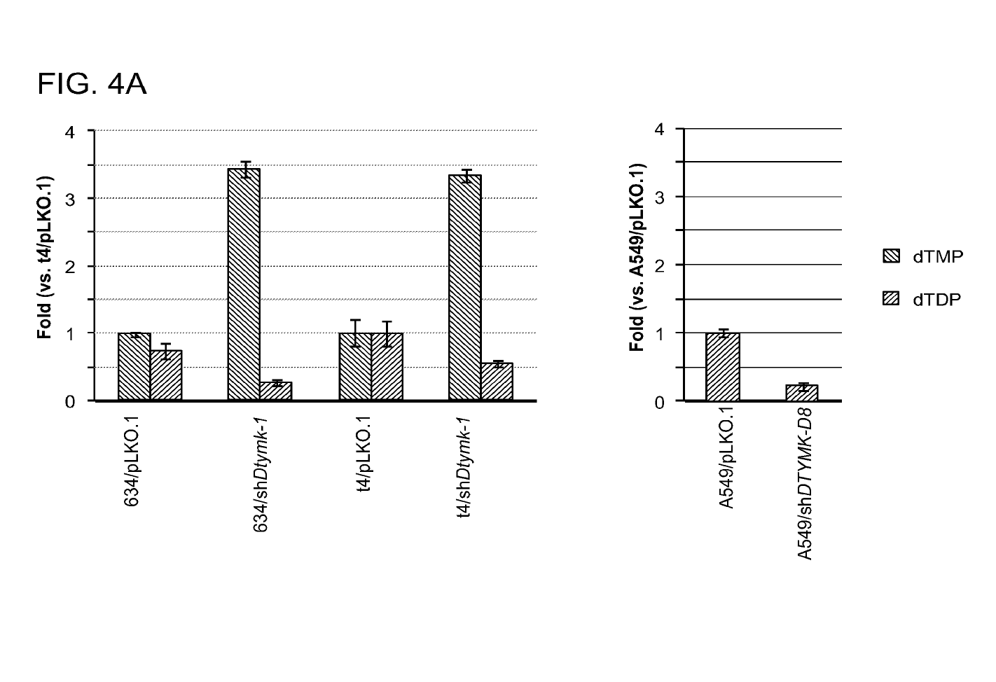

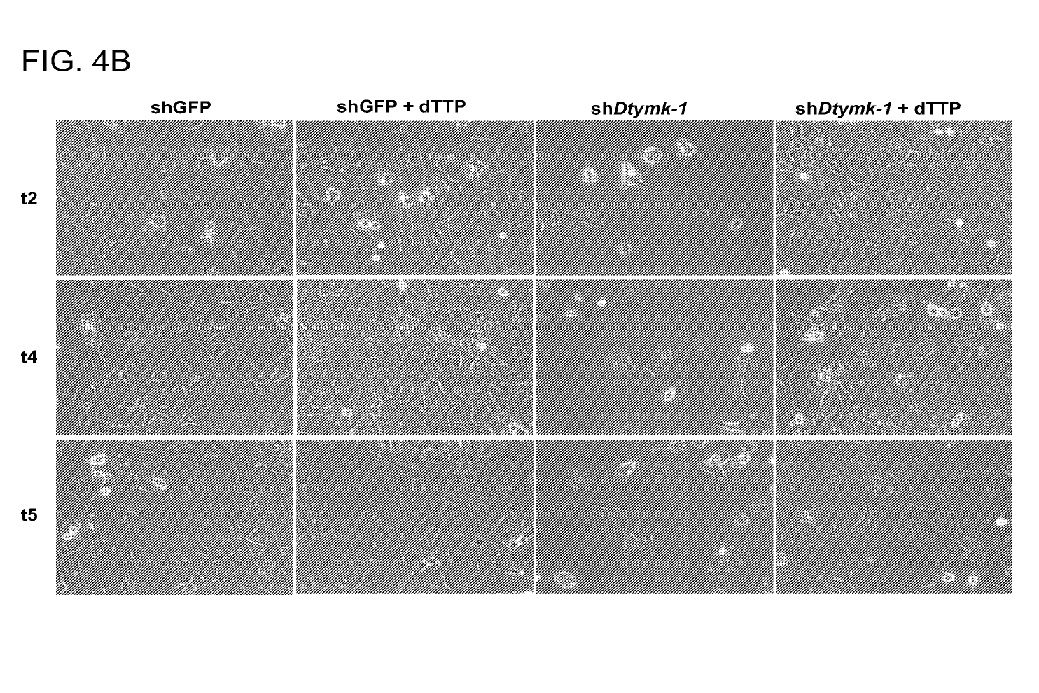

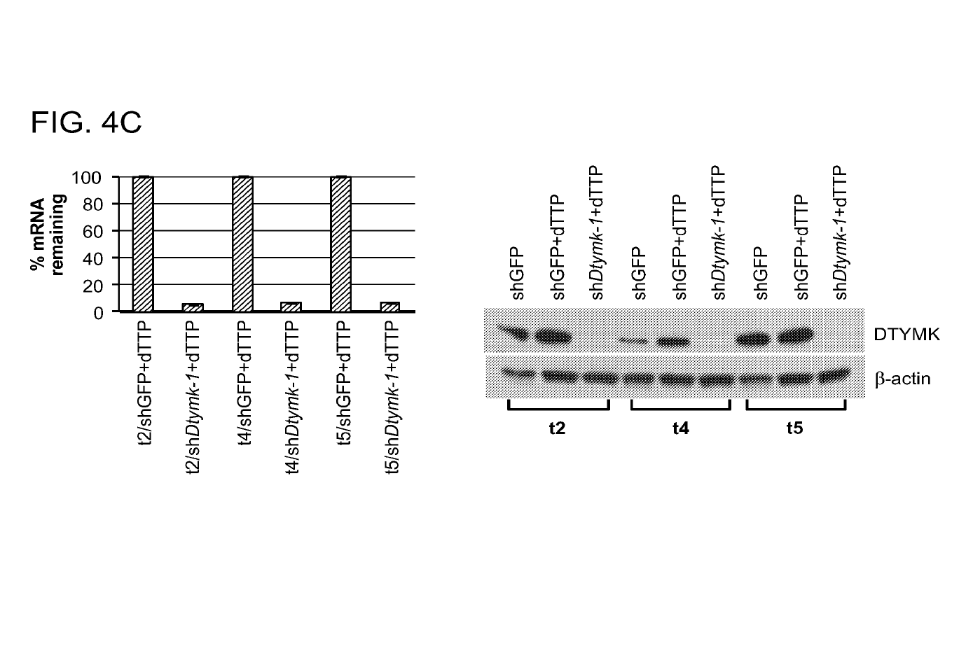

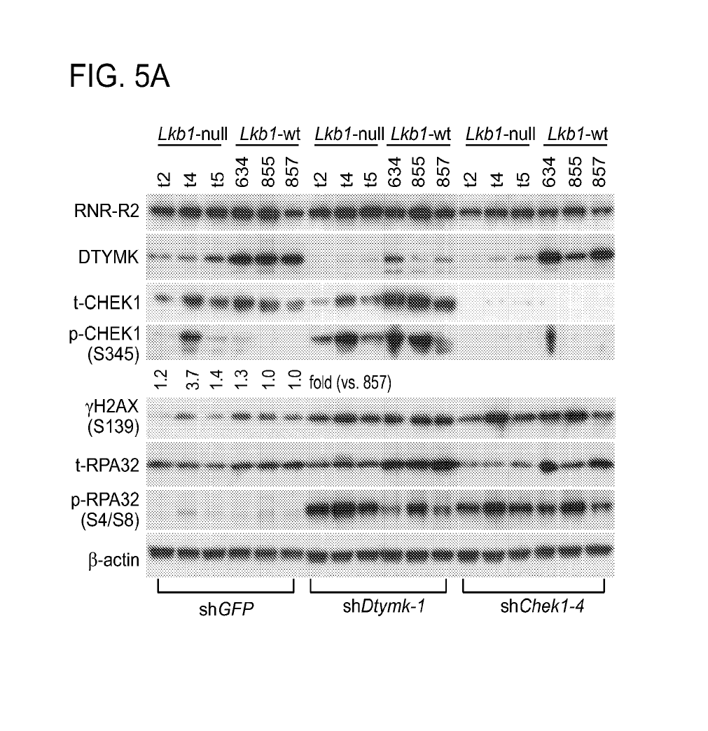

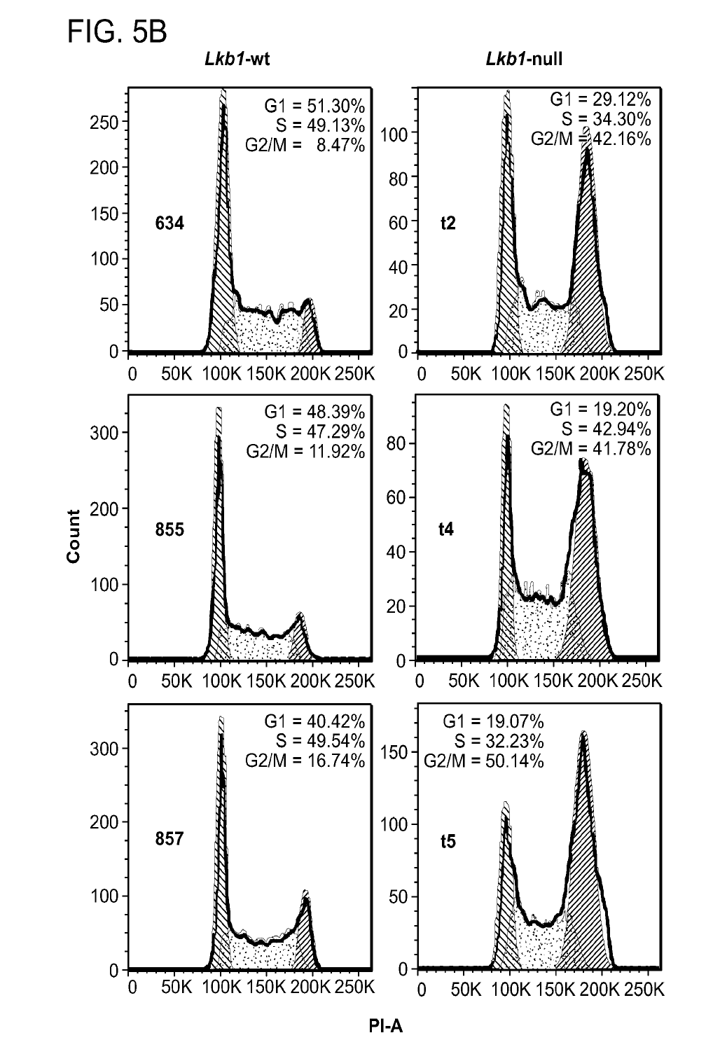

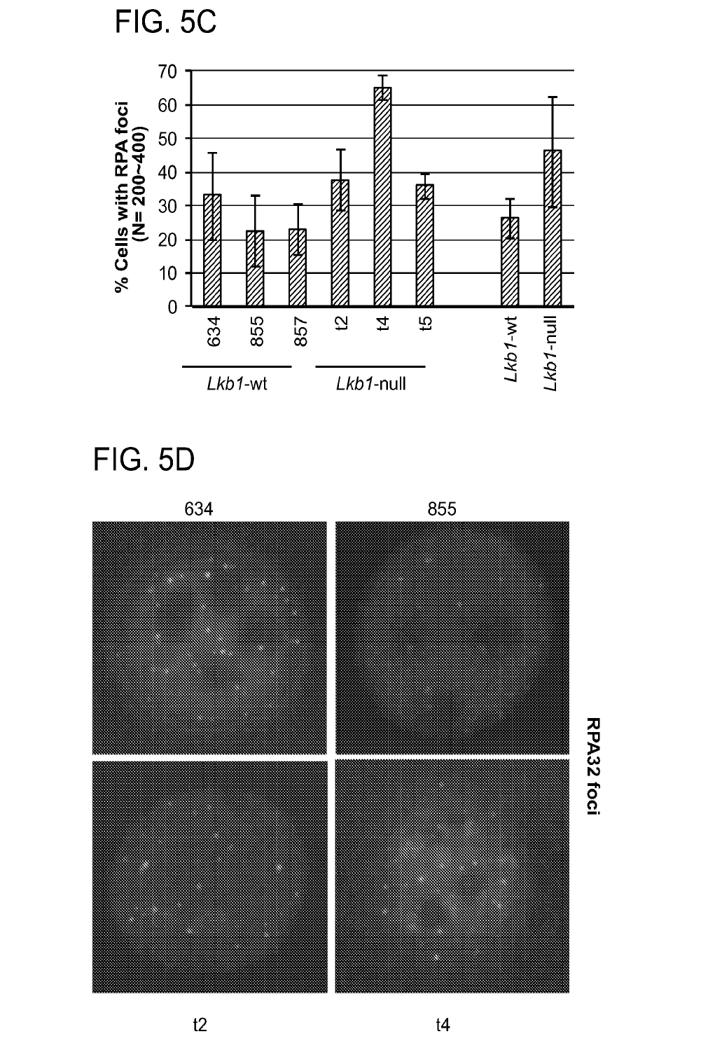

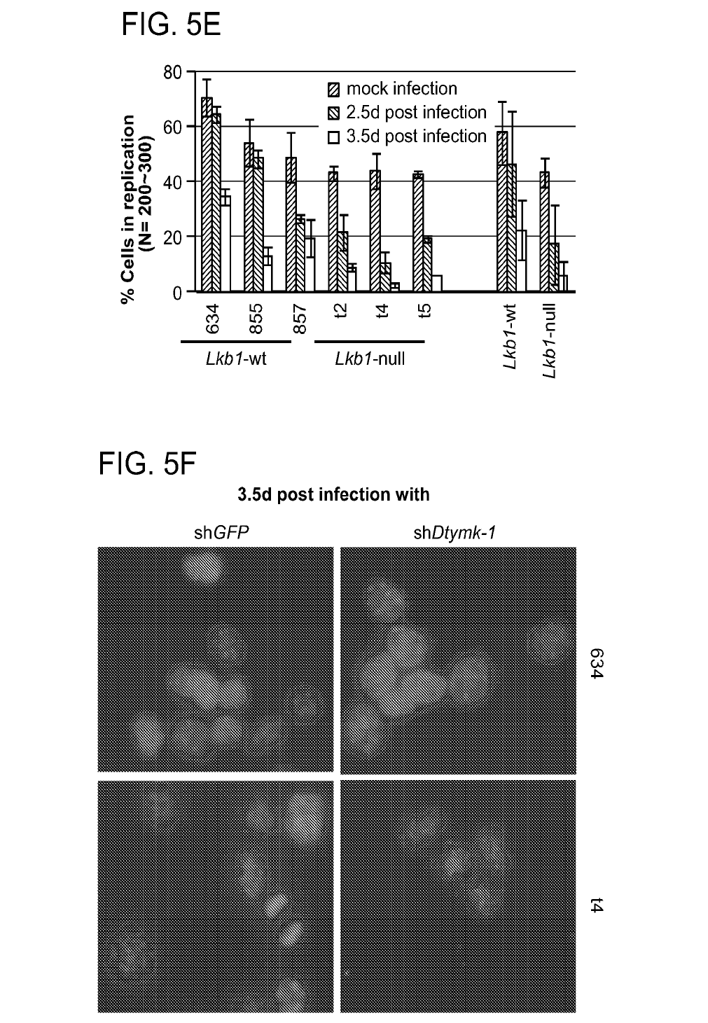

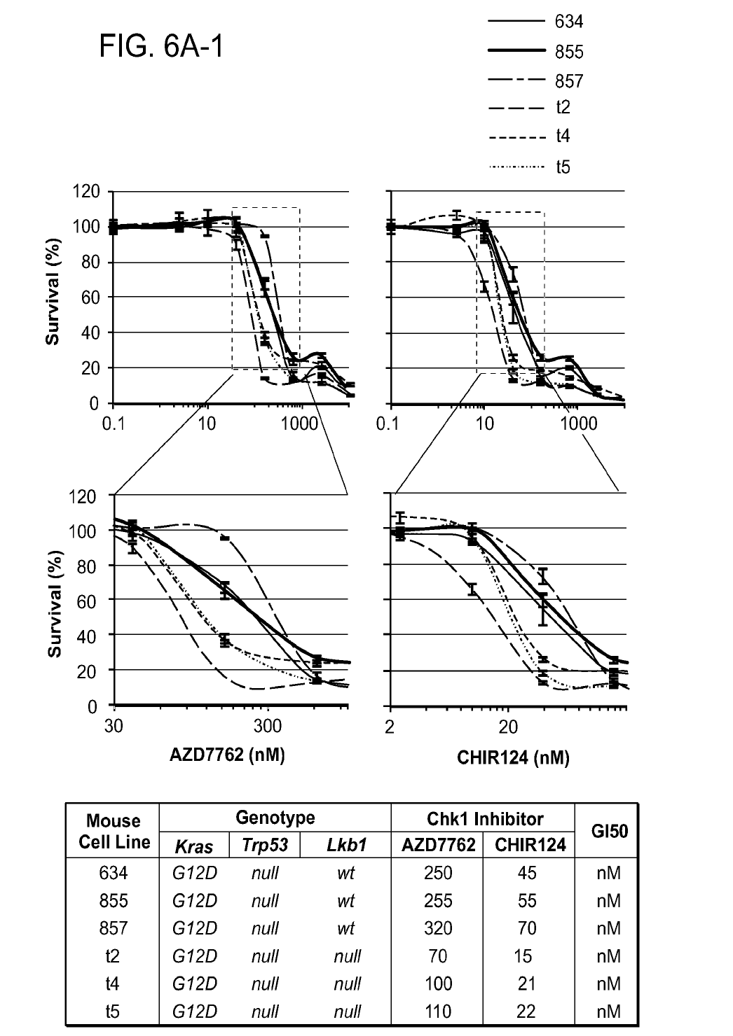

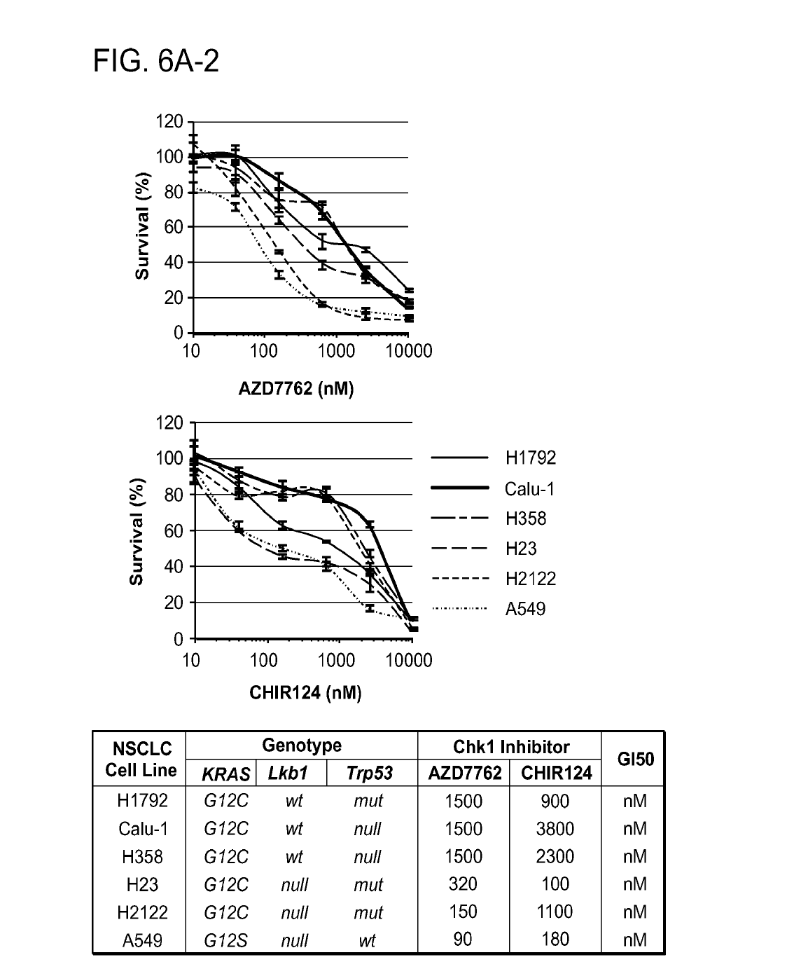

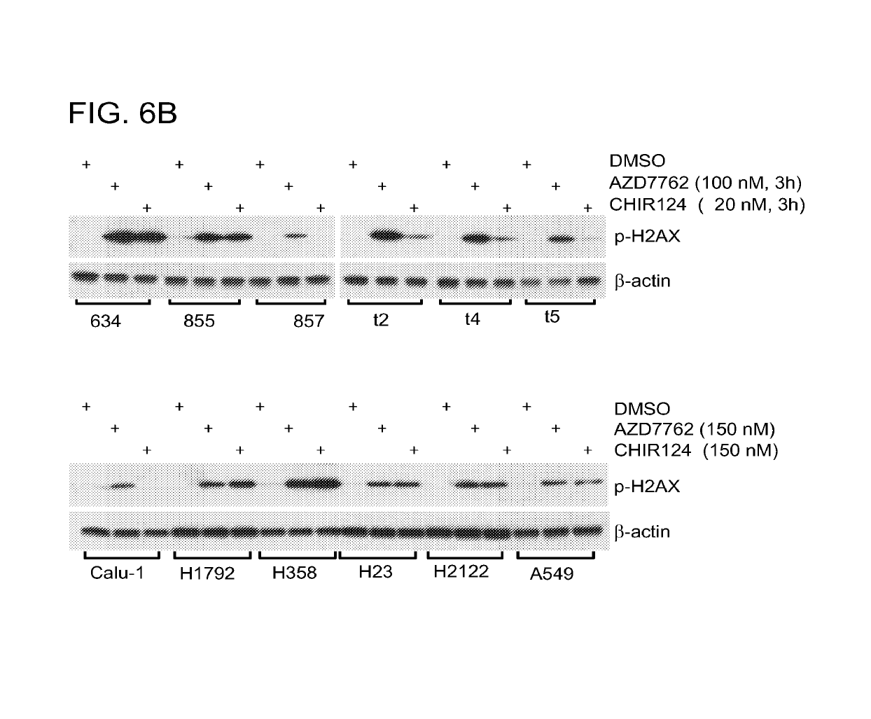

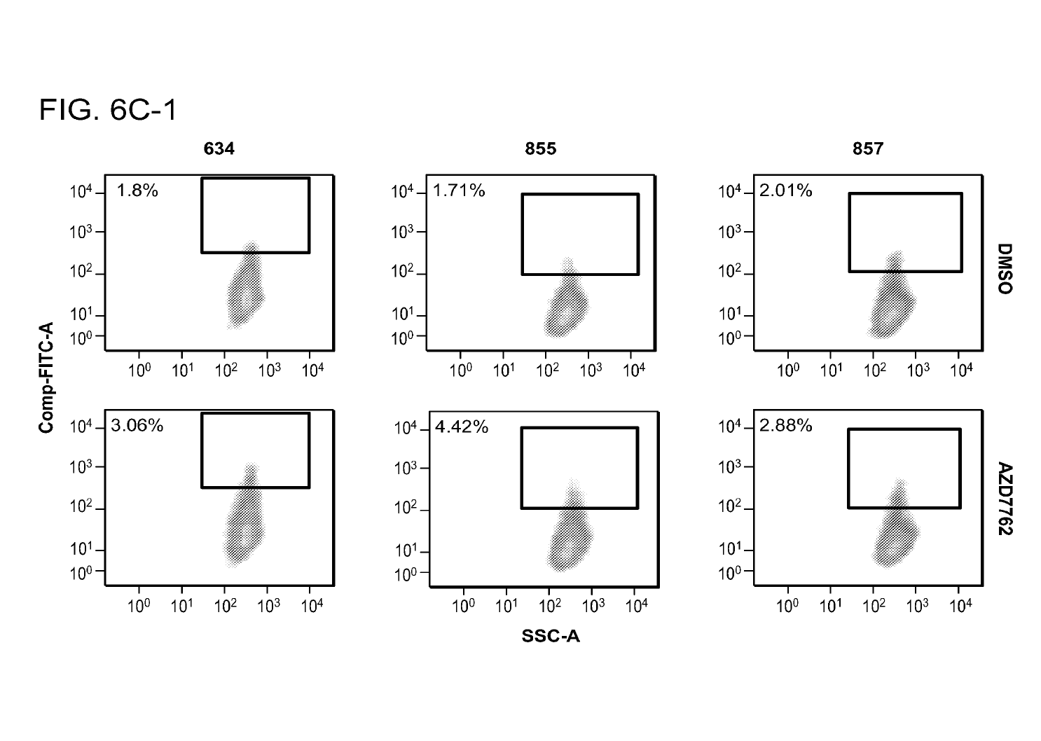

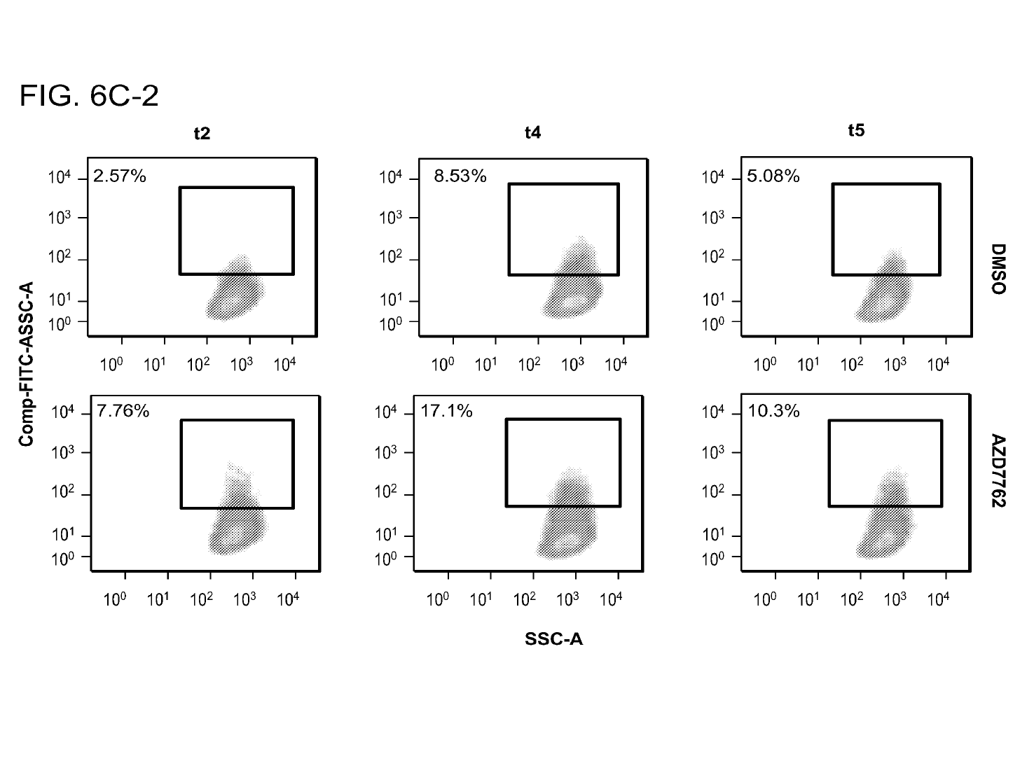

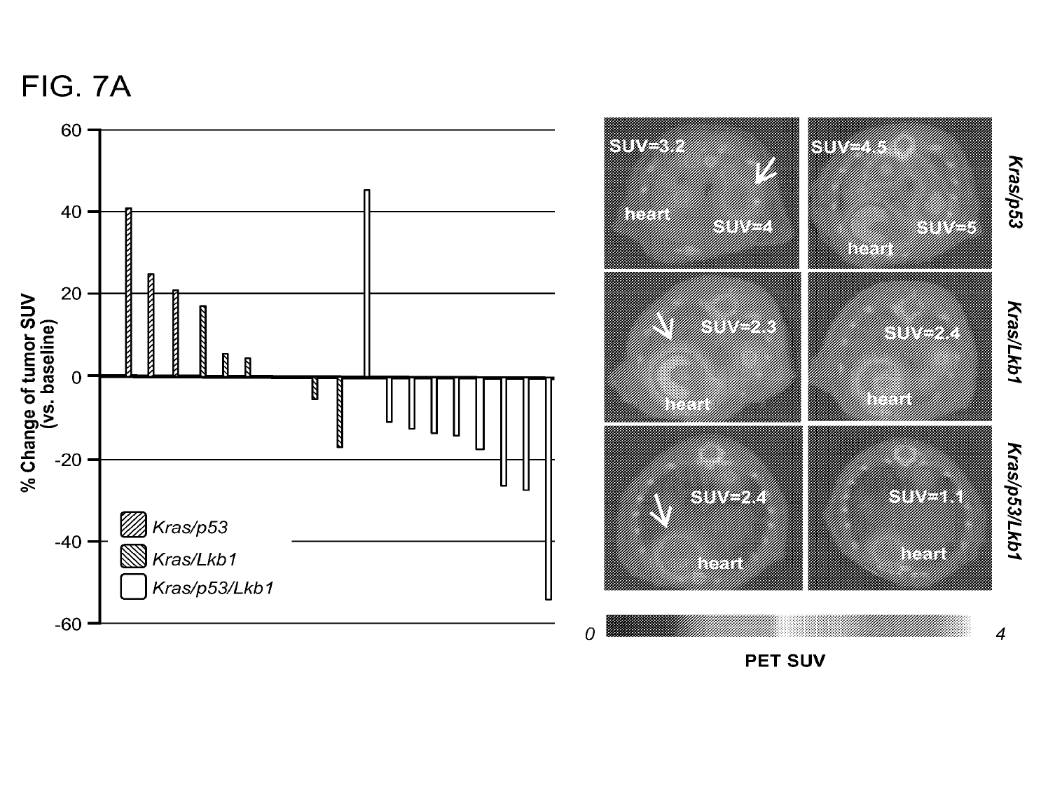

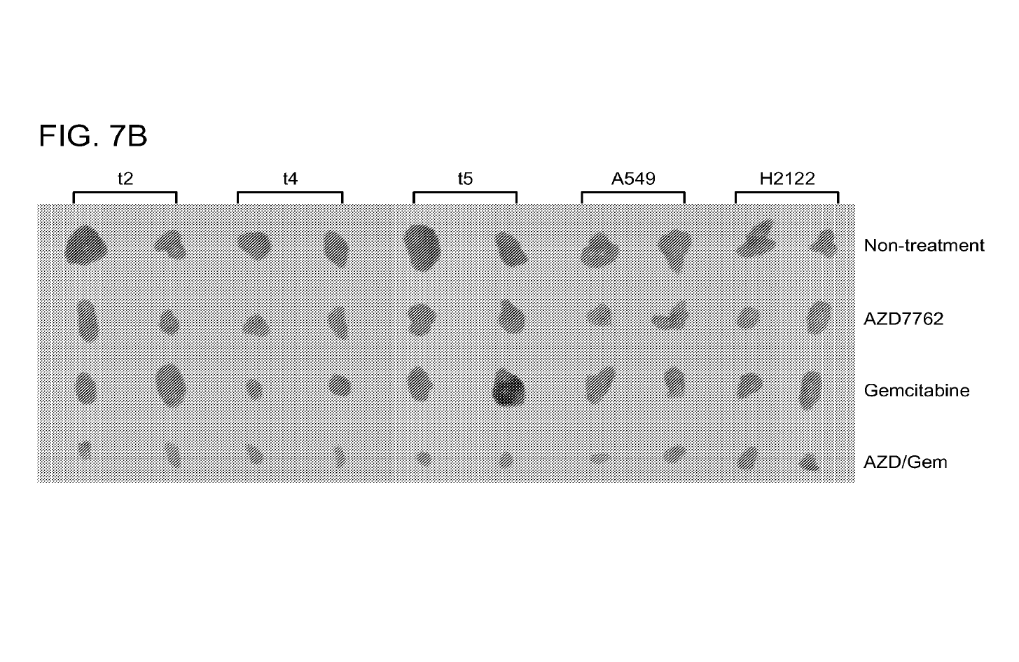

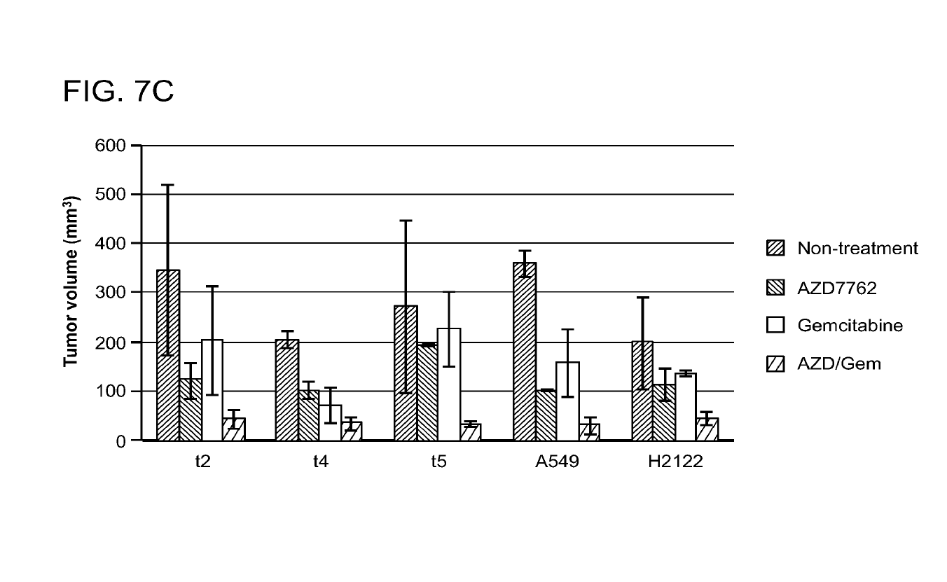

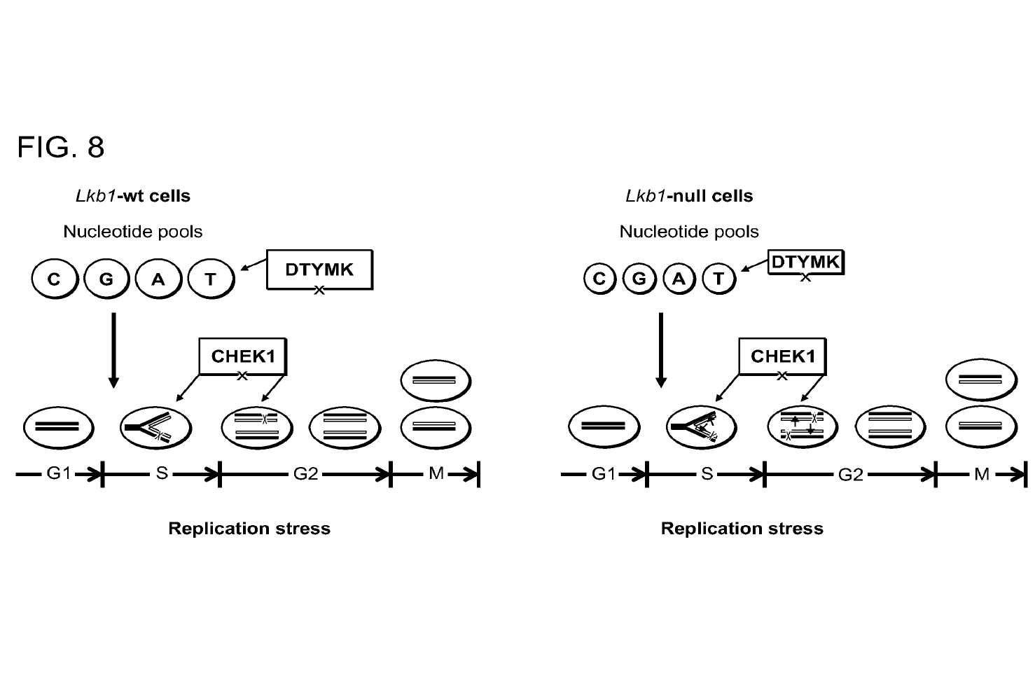

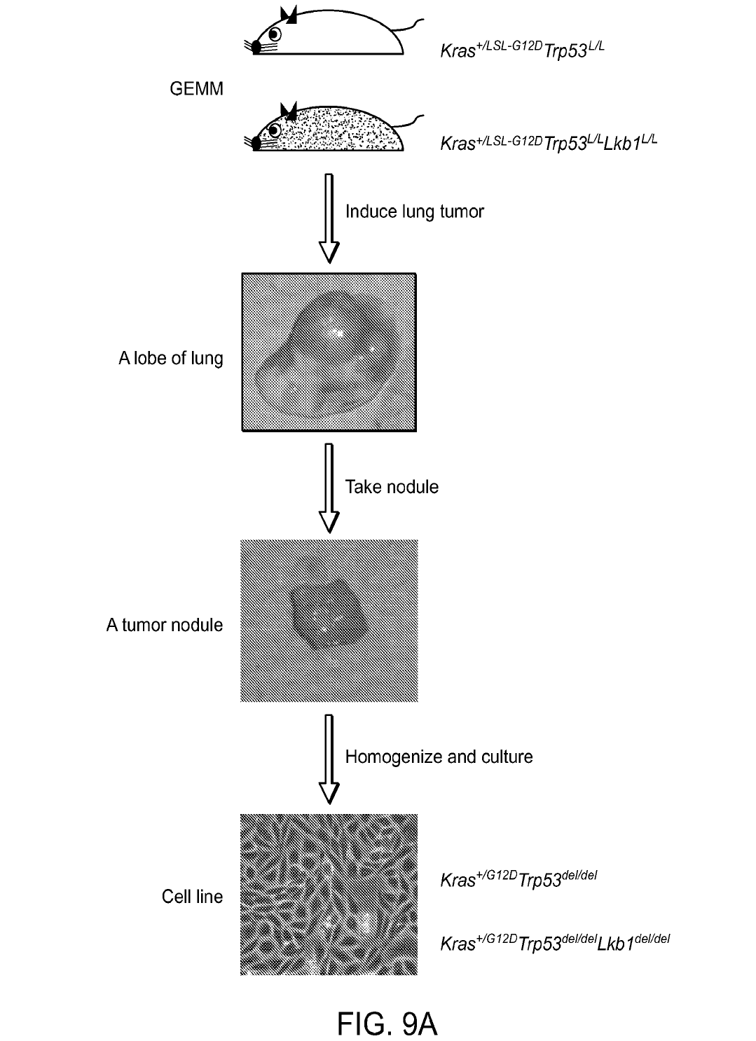

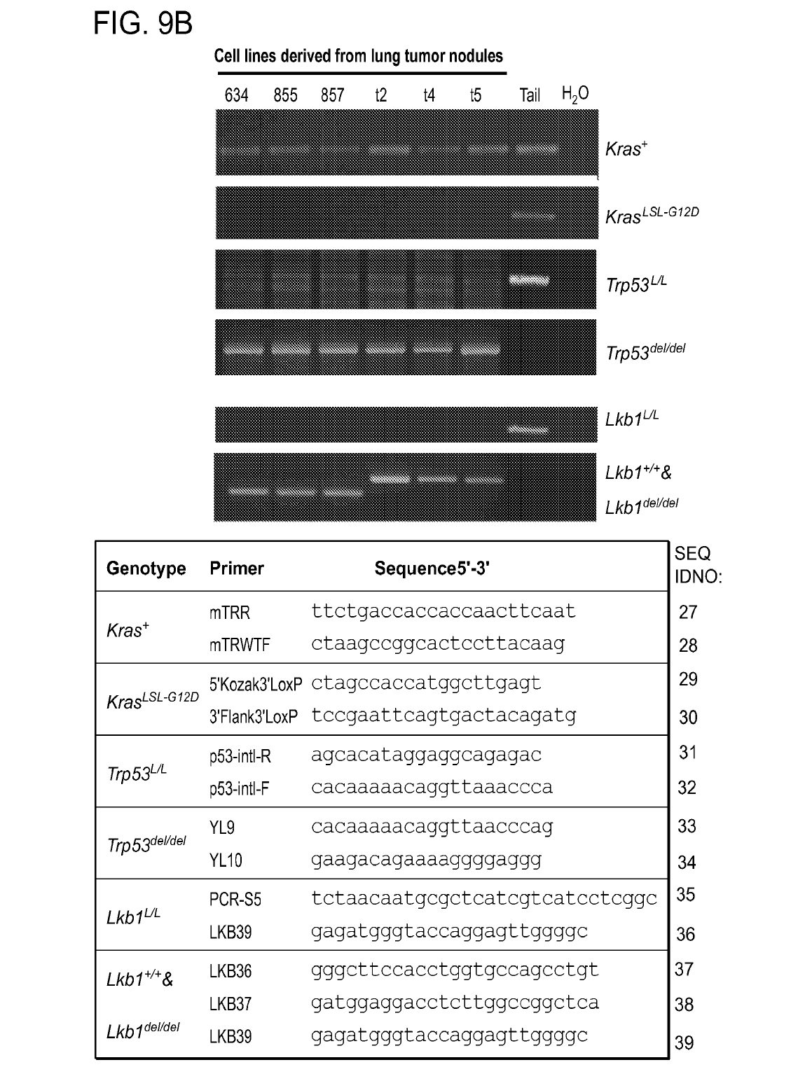

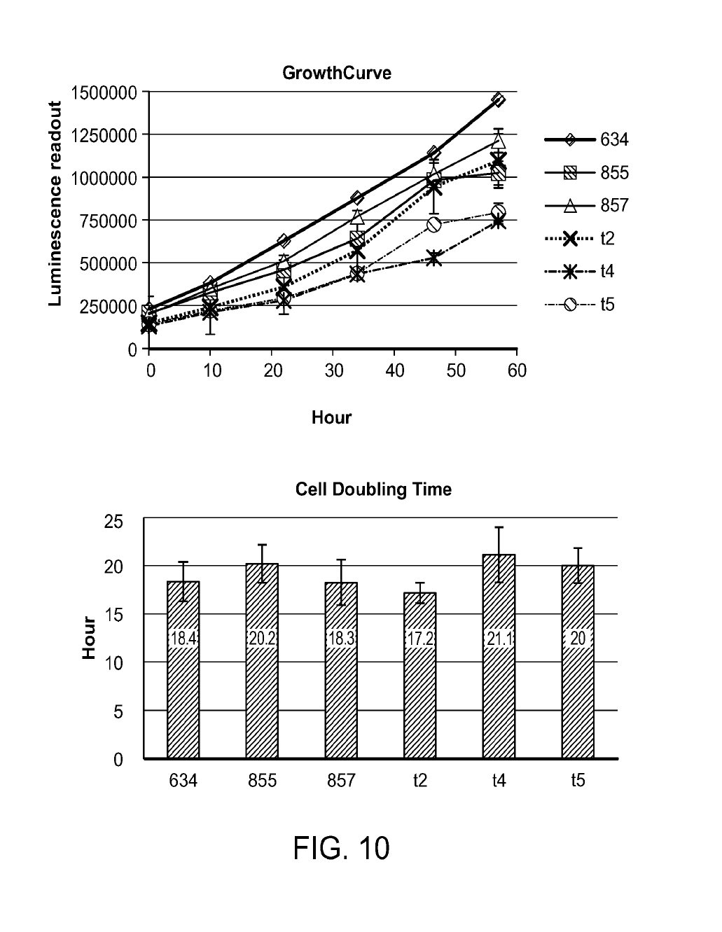

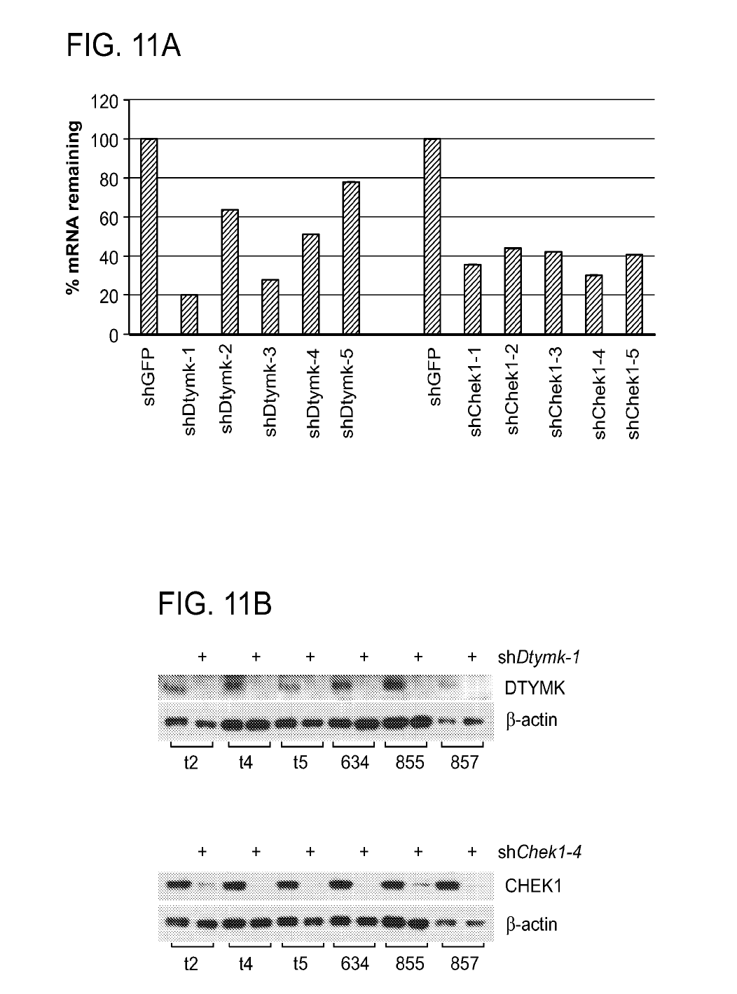

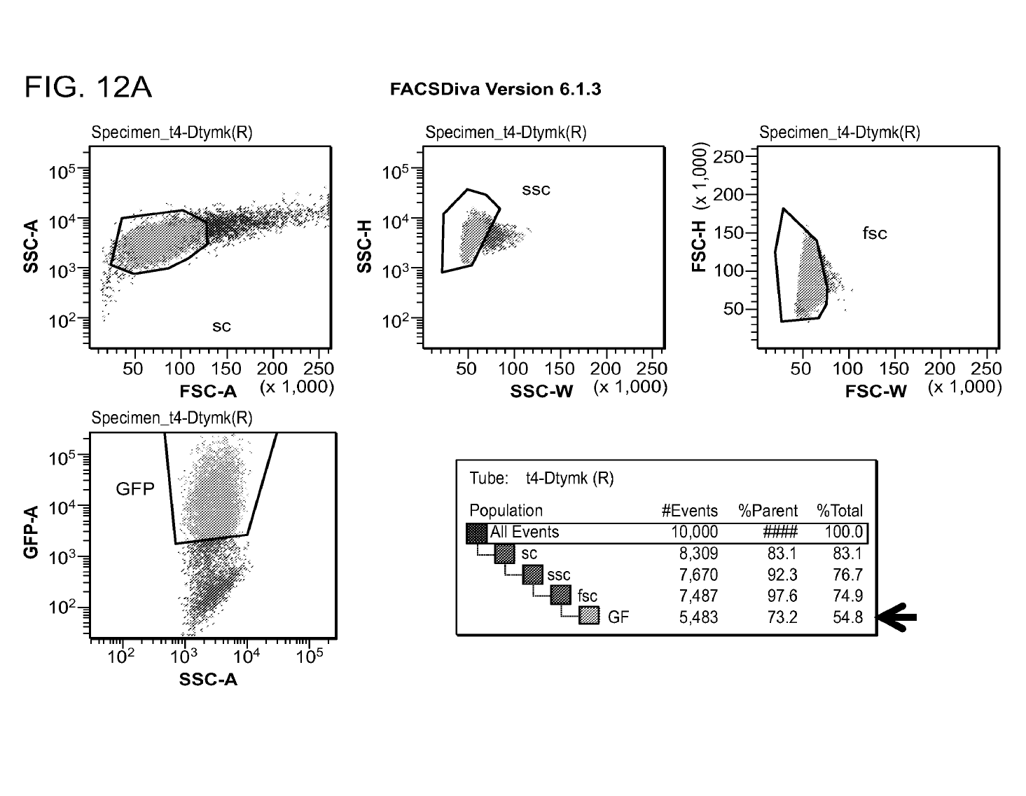

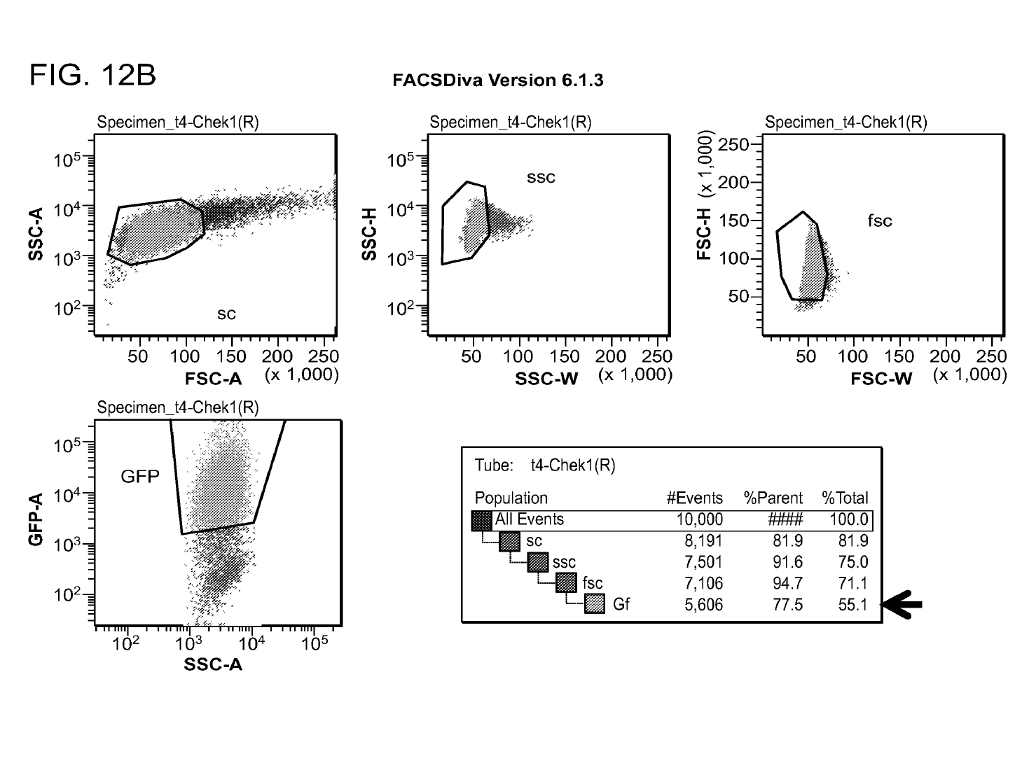

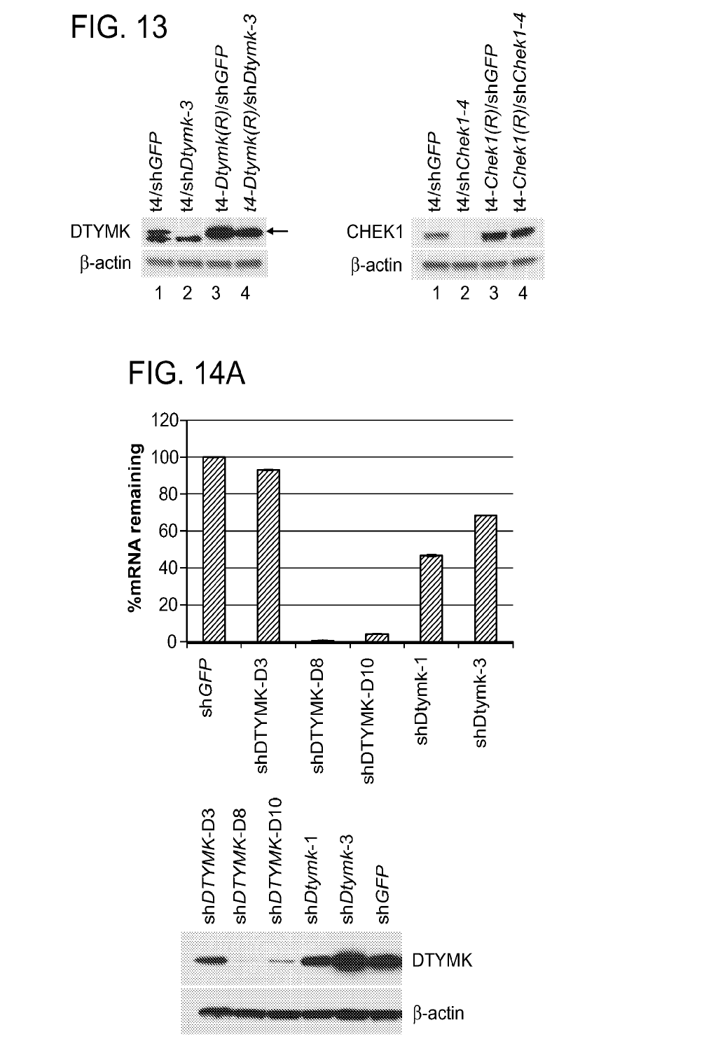

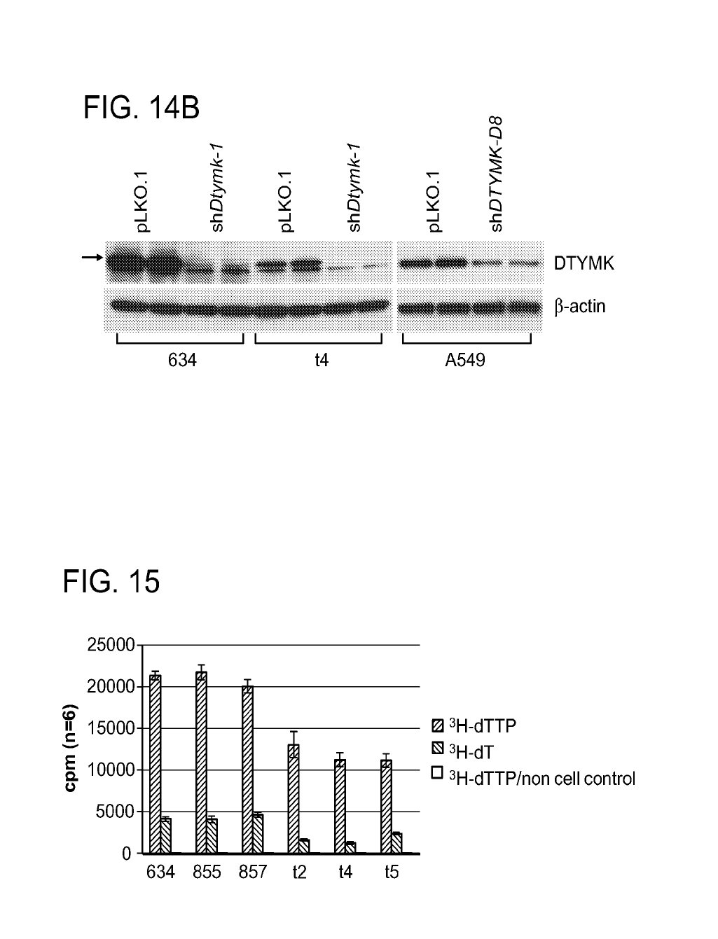

To identify Lkb1-null-selective essential genes, a synthetic lethal screen was performed using a pooled 40K murine shRNA lentiviral library for each of the Lkb1-wt and Lkb1-null cell lines described above. Relative abundance of shRNAs in each cell line sample was determined by deep-sequencing analysis, and for every shRNA, a log 2 Fold Change (log 2FC) value was calculated from the difference in relative abundance at a late time point after infection versus the initial shRNA-infected sample. An unsupervised hierarchical clustering analysis of the ranked hairpins from the triplicate pooled shRNA library screens of Lkb1-wt and Lkb1-null mouse cancer cells is shown in FIG. 1A. The blue-color in the top-right corner represents genes for which the abundance of shRNAs is significantly reduced in all 3 Lkb1-null cultures, suggesting a specific effect in the inhibition of Lkb1-null cell growth (FIG. 1A). The ranked hairpins were collapsed by using two methods, a RIGER analysis (KS t-test based statistics) and a weighted second best analysis to rank genes that selectively impaired proliferation/viability in Lkb1-null cells. A union of 344 genes, identified by the top 100 individual hairpins for 88 genes (Table 1) and the top 200 genes from both the KS (Table 2) and weighted second best (Table 3), was nominated as the initial prioritized list (FIG. 1B). 340 shRNAs, targeting 70 candidate genes from this prioritized list, were chosen for validation (Table 4). The 70 genes consisted of the top 10 candidates from the KS analysis, as well as 60 others involved in a range of biological processes in an attempt to represent all biological categories in the validation process. Validation was performed in an array format with an assay of relatively short duration (5 days post infection) compared to the primary pooled screen (28 days), which should be a more stringent selection, as it required the anti-proliferative effects to manifest in a short time period. The validation identified 13 genes that displayed 2 or more hairpins with a significant growth disadvantage in the Lkb1-null cells (Table 5). Dtymk, Check1, and Pdhb are the top 3 candidate genes, each with 3 hairpins that scored in the validation assay (FIG. 1C).

### Example 4

### Complementary Analyses Also Implicate DTYMK and Chek1 as Critical Genes in Lkb1-Null Cells

To provide additional, orthogonal information on potential selective targets in Lkb1-null cells, a high-throughput screen of a protein kinase inhibitor-enriched small molecule library was performed in parallel. The library comprised approximately 1,000 small molecule kinase inhibitors, including protein kinase inhibitors in preclinical studies and those approved for clinical use, as well as in-house tool-like pharmacophore kinase inhibitors, which in aggregate target a significant fraction of the kinome. As shown in FIG. 2A, at a fixed dose of 660 nM for all compounds, 11 compounds inhibited the growth of both Lkb1-wt and Lkb1-null GEMM cell lines. Some kinase inhibitors had greater growth inhibitory effects on the Lkb1-null than Lkb1-wt cells in this assay, including Kin177 (AZD7762), which inhibits CHEK1 kinase, a candidate gene identified in the shRNA screen.

LKB1 is reported to be involved in metabolic reprogramming (Gurumurthy et al., 2010; Jansen et al., 2009), therefore the metabolic profile of Lkb1-wt and Lkb1-null cells was assessed. A set of 58 metabolites, including nucleotide metabolites IMP, AMP, ADP, GMP, dGMP, UMP, UDP, CDP, dCDP, and dTDP, was discovered that were present at consistently lower levels in Lkb1-null cells (FIG. 2B). Pathway enrichment analysis demonstrated that metabolites in both purine and pyrimidine metabolism were significantly reduced in Lkb1-null compared to Lkb1-wt cells (FIG. 2B, P=3.5×10⁻⁷ and 3.4×10⁻⁵, respectively). In particular, Lkb1-null cells had a lower level of dTDP, which is the product of deoxythymidylate kinase (DTYMK), also known as thymidylate kinase (TMPK) or dTMP kinase (FIG. 2C). Dtymk is one of the candidate genes with strongest synthetic lethality towards Lkb1-null cells in the RNAi screen. Despite lower nucleotide levels, Lkb1-null cells have a similar doubling time as Lkb1-wt cells (FIG. 10), suggesting that although DNA biosynthesis can still match cell proliferation, the Lkb1-null cells may be more sensitive to changes in DTYMK activity. Collectively, these two independent sets of data suggest that Dtymk and Chek1 are essential genes in the Lkb1-null context, and therefore have potential as important targets in Lkb1-null lung cancer.

### Example 5

### Dtymk and Chek1 are Synthetic Lethal Genes Selectively Required for Lkb1-Null Cell Proliferation In Vitro and In Vitro

To determine the knockdown efficiency of shDtymk and shChek1, a set of 5 shRNAs for Dtymk or Chek1 was packaged individually and transduced into Lkb1-wt 634 cells (Table 6). After 2-3 days post puromycin selection, Western blot analysis of the cells showed that at least two shRNAs from each set knocked down DTYMK or CHEK1 to undetectable levels (FIG. 3A and FIG. 11A). This data confirmed that the shDtymks and shChek1s do indeed target Dtymk and Chek1, respectively.

To investigate if reduced expression of Dtymk or Chek1 inhibited cell growth in vitro, proliferation assays were performed in Lkb1-wt (634, 855, and 857) and Lkb1-null (t2, t4, and t5) cells transduced with the top two shRNAs for Dtymk or Chek1. Both shDtymk-1 and shDtymk-3 inhibited Lkb1-wt and Lkb1-null cell growth compared to shGFP control, but the inhibition was stronger in Lkb1-null cells (FIG. 3B). Similarly, both shChek1-1 and shChek1-4 inhibited Lkb1-null cell growth more strongly than the growth of Lkb1-wt cells, except for Lkb1-wt 855 cells, which showed inhibition similar to that of the Lkb1-null cells (FIG. 3B). Depletion of DTYMK and CHEK1 was confirmed by Western blot (FIG. 11B).

To investigate whether reduced expression of Dtymk or Chek1 inhibited tumor development in vivo, Lkb1-wt (634 and 857) and Lkb1-null (t2 and t4) cells transduced with pTetOn-shGFP, pTetOn-shDtymk-3, or pTetOn-shChek1-4 were implanted into athymic nude mice. Consistent with the in vitro proliferation assay, after doxycycline treatment for 3 weeks, Lkb1-null tumors expressing shDtymk or shChek1 grew significantly slower than Lkb1-null tumors expressing shGFP and Lkb1-wt tumors (FIGS. 3C and 3D).

To determine whether overexpression of a shRNA-resistant cDNA allele of Dtymk or Chek1, Dtymk(R) or Chek1(R), could rescue the Dtymk or Chek1 knockdown phenotype, Lkb1-null t4 cells were transduced with either pCDH-Dtymk(R) or pCDH-Chek1 (R) that both co-express GFP, and the resulting t4-Dtymk(R) and t4-Chek1(R) cells were collected by FACS. Proliferation assays showed that growth of t4-Dtymk(R) and t4-Chek1 (R) cells upon shRNA transduction was dramatically increased, but not fully restored to the rates of t4/shGFP cells (FIG. 3E). Further FACS analysis of the t4-Dtymk(R) and t4-Chek1(R) cells used in the rescue assay showed that only approximately 55% of the population was either Dtymk(R)/GFP or Chek1(R)/GFP positive (FIG. 12), providing one explanation for the significant, although incomplete rescue. Depletion of endogenous DTYMK and CHEK1 and overexpression of exogenous resistant DTYMK and CHEK1 in the rescue assay were confirmed by Western blot (FIG. 13). Collectively, these data suggest that Dtymk and Chek1 are selective synthetic lethal genes of Lkb1-null cells.

### Example 6

### Knockdown of Dtymk/DTMK Alters Pyrimidine Metabolism

DTYMK catalyzes the phosphorylation of dTMP to form dTDP, and it is the first merged step of both the de novo and salvage pathways in the production of dTTP nucleotides for DNA synthesis. (FIG. 2C). It was expected that Dtymk knockdown would inhibit this pathway and lead to accumulation of the substrate dTMP and decrease of the product dTDP. To test this, Lkb1-wt 634 and Lkb1-null t4 cells were transduced with shDtymk-1. Metabolite analysis of the cells revealed the expected significant increase in dTMP and moderate decrease in dTDP levels (FIG. 4A), indicating that Dtymk was depleted to a level sufficient to reduce enzyme activity in both Lkb1-wt and Lkb1-null cells. The knockdown of Dtymk was confirmed by Western blot (FIG. 14). Furthermore, knockdown of DTYMK in human LKB1-deficient NSCLC A549 cells also reduced dTDP levels (FIG. 4A and FIG. 14). This finding indicates that DTYMK is a major source of dTDP in human lung cancer cells and underscores the importance of this gene in cancer cell proliferation, as dTDP is required for production of dTTP for DNA synthesis. Collectively, these results indicate that knockdown of Dtymk/DTYMK in both mouse and human lung cancer cells sufficiently lowers protein expression and enzyme activity to significantly inhibit pyrimidine metabolism.

### Example 7

### dTTP Rescues shDtymk Growth Phenotype

Next it was determined whether adding dTTP to the media could rescue cell death after Dtymk knockdown. Three Lkb1-null cell lines were transduced with shGFP or shDtymk-1 and then selectively cultured in puromycin medium in the presence or absence of 150 μM dTTP for 3 days (Taricani et al., 2010). The amount of dTTP used in the rescue assay was determined not to be toxic as the shGFP-transduced cells grew normally in the same dTTP medium (FIG. 4B). All shDtymk-transduced Lkb1-null cells grew poorly without additional dTTP; however, with exogenous dTTP, they grew as well as shGFP-transduced cells (FIG. 4B). Expression of DTYMK in the shDtymk+dTTP cells, determined by qPCR and Western blot, was not detectable, suggesting that growth was dependent on the addition of dTTP to the culture medium (FIG. 4C). Expression of DTYMK in the shDtymk-1 cells was not determined because the remaining cells were too few for RNA and protein extraction (FIG. 4B). Incorporation of the exogenous ³H-dTTP into genomic DNA confirmed that the radiolabeled dTTP had passed through the cell membrane (FIG. 15). Therefore, rescue of the shDtymk growth-deficient phenotype by exogenous dTTP provides additional evidence that the effect of the shRNA is on-target, and demonstrates that Dtymk is required for its enzymatic activity in these cells.

### Example 8

### Lkb1-Null Cells are More Prone to DNA Damage than Lkb1-Wt Cells

To understand possible mechanisms behind the synthetic lethal interaction between Lkb1-null and deletion of Dtymk or Chek1, the replication stress in Lkb1-wt and Lkb1-null cells was characterized, starting with a set of Western blot analyses. Ribonucleotide reductase (RNR) catalyzes the formation of deoxyribonucleotide (dADP, dGDP, dCDP, and dUDP) from ribonucleotide (ADP, GDP, CDP, and UDP), whereas dTDP is synthesized from dTMP by DTYMK (Elledge et al., 1992; Su and Sclafani, 1991). Hu et al recently reported that in cancer cells expressing high levels of the RNR-R2 subunit and deficient in DTYMK, dUTP is misincorporated into DNA in place of dTTP (Hu et al., 2012). Therefore, the expression of RNR-R2 and DTYMK in Lkb1-null and Lkb1-wt cells was investigated. As shown in FIG. 5A, Lkb1-null and Lkb1-wt cells have similar RNR-R2 expression, but Lkb1-null cells have much lower DTYMK expression, enabling a cellular state in which dUTP is misincorporated into DNA. It has been established that if two dUTP nucleotides are misincorporated in proximity to each other, uracil-DNA glycosylase-mediated DNA nucleotide excision repair will result in DNA double-strand breaks (DSBs) (Marenstein et al., 2004). As such, densitometric analysis of phospho-CHEK1 (p-CHEK1) Western blot revealed slightly increased basal p-CHEK1 in Lkb1-null compared to Lkb1-wt cells (1.2, 3.7, 1.4 vs. 1.3, 1.0, 1.0). In addition, flow cytometry analysis of asynchronous Lkb1-wt and Lkb1-null cells revealed a large 4N peak in Lkb1-null cells (FIG. 5B). Because Lkb1-null and Lkb1-wt cell lines have a similar doubling time, the 4N peak suggests a G2 delay for repairing damaged DNA generated during replication in Lkb1-null cells. Collectively, these data support that Lkb1-null cells have higher levels of baseline DNA damage than Lkb1-wt cells.

Next, the baseline level of γH2AX in Lkb1-wt and Lkb1-null cells was determined. γH2AX is a selective marker of DNA DSBs, acting at DNA DSB sites to recruit other DNA damage response proteins for repair (Liu et al., 2008; Rogakou et al., 1998; Wu et al., 2005). Although the data shown above indicated that Lkb1-null cells have higher levels of DNA damage, Western blot revealed that Lkb1-wt and Lkb1-null cells have similar levels of baseline γH2AX, suggesting the levels of DNA DSBs are similar (FIG. 5A). These data suggest that DNA DSBs are not responsible for the large 4N peak in Lkb1-null cells. Upon knockdown of Dtymk, the phosphorylation of both H2AX and CHEK1 increased, suggesting more DNA DSBs in both Lkb1-wt and Lkb1-null cells (FIG. 5A). These data further suggest that DTYMK and CHEK1 are functionally related.

Replication protein A (RPA) associates with and stabilizes single-stranded DNA during DNA replication, recombination, and repair (Wold, 1997). RPA32, the 32 kDa subunit of RPA, is phosphorylated upon DNA damage or replication stress by kinases including ATM, ATR, and DNA-PK (Zou et al., 2006). Western blot revealed slightly higher total RPA32 (t-RPA32) expression in Lkb1-wt cells (FIG. 5A), whereas indirect immunofluorescence microscopy revealed a slightly higher proportion of Lkb1-null than Lkb1-wt cells showing RPA foci (FIGS. 5C and 5D), suggesting more DNA damage in Lkb1-null cells. Upon knockdown of Dtymk or Chek1, phosphorylation of RPA32 increased in both Lkb1-wt and Lkb1-null cells (FIG. 5A), indicating that depletion of Dtymk or Chek1 leads to DNA damage or replication stress in both genotypes. Notably, a significantly larger increase of phospho-RPA32 is observed in the Dtymk-depleted Lkb1-null versus Dtymk-depleted Lkb1-wt cells (FIG. 5A). These data further suggest Lkb1 loss sensitizes cells to Dtymk deletion-induced DNA damage and replication stress, as equivalent depletion of Dtymk in Lkb1-null and Lkb1-wt cells leads to more robust DNA damage in the Lkb1-null cell lines. In addition, we noticed the expression of t-RPA32 increased in Lkb1-wt cells upon knockdown of Dtymk or Chek1, and stronger t-RPA32 correlated to weaker p-RPA32, except in 855 cells (FIG. 5A).

### Example 9

### DNA Replication is More Sensitive to Dtymk Knockdown in Lkb1-Null than in Lkb1-Wt Cells

The lower expression of DTYMK in Lkb1-null cells causes the cells to be in jeopardy of DNA damage. To investigate how further knockdown of Dtymk and the consequent decrease in the dTTP pool could affect DNA metabolism, IdU pulse-labeling in Lkb1-wt and Lkb1-null cells was performed 2.5 and 3.5 days post-transduction with shDtymk-1. After indirect immunostaining with anti-BrdU, fluorescence microscopy revealed that the proportion of cells labeled with IdU dropped dramatically upon Dtymk knockdown. As shown in FIG. 5E, the proportions of labeled Lkb1-wt cells at day 0, 2.5, and 3.5 post infection were 57.7%, 46.3%, and 22.3% (mean), dropping 61.2% in 3.5 days, and the proportions of labeled Lkb1-null cells were 43.1%, 17.2%, and 5.8% (mean), dropping 86.5% in 3.5 days. Over these 3.5 days, fewer and fewer of the attached cells were labeled, and most of the unlabeled nuclei in Lkb1-null cells were deformed and fragmented, suggesting thymineless death (Kuong and Kuzminov, 2012). The representative images of Lkb1-wt and Lkb1-null cells co-stained for IdU and DAPI are shown (FIG. 5F). In addition, an overall lower proportion of labeled Lkb1-null than Lkb1-wt cells (43.1% vs. 57.7%) was observed, which may be related to the lower dNTP levels in Lkb1-null cells. Collectively, these data suggest that DNA replication in Lkb1-null lines is more sensitive to Dtymk knockdown than in Lkb1-wt lines.

### Example 10

### Lkb1 Mutant Cells are Hypersensitive to Chek1 Inhibition

Multiple small molecule inhibitors of CHEK1 have been developed and are suitable tools to evaluate Lkb1-null cell sensitivity to CHEK1 inhibition. Two specific ATP-competitive small molecule inhibitors of CHEK1, AZD7762 and CHIR124 (Tse et al., 2007; Zabludoff et al., 2008), were selected to validate the importance of CHEK1 function in Lkb1-null cell growth and survival. It was determined that both AZD7762 and CHIR124 inhibited Lkb1-null cells 3-fold stronger than Lkb1-wt cells with 50% growth inhibition (GI50) concentrations of 90 nM versus 275 nM (mean) for AZD6244 and 19 nM versus 56 nM for CHIR124, respectively (FIG. 6A). These studies were extended to human cancer cell lines harboring similar mutation profiles: KRAS activation versus KRAS activation/LKB1-deficient with and without TP53 mutations. Consistent with the results in the mouse lung cancer cell lines, the LKB1-deficient NSCLC cell lines H23, H2122, and A549 showed greater growth inhibition in response to CHEK1 inhibitors than the LKB1-wt NSCLC cell lines H1792, Calu-1, and H358 (FIG. 6A). Interestingly, A549, a TP53-wt cell line, showed the greatest sensitivity to the CHEK1 inhibitors. These data may suggest that TP53 loss is not required for the synthetic lethal interaction between the inhibition of CHEK1 and LKB1 loss. Although the human cell lines were overall less sensitive to the drugs tested, the difference between the LKB1-deficient and LKB1-wt lines was greater than for the mouse cell lines (5-20 fold versus 3 fold), indicating a higher relative selectivity. Western blot confirmed that AZD7762 and CHIR124 treatments for 3 h at the G150 concentration induced phosphorylation of H2AX (FIG. 6B). These data, in agreement with the Chek1 knockdown, suggest that CHEK1 inhibition leads to significant DNA DSBs, likely contributing to reduced cell growth.

Next, the mechanism behind why the inhibition of CHEK1 killed more Lkb1-null than Lkb1-wt cells was investigated. Current characterization of Lkb1-null cells has suggested more DNA damage, making cells more dependent on the DNA repair system. After AZD7762 treatment for 3 h, Western blot did not reveal a noticeable difference on γH2AX between Lkb1-null and Lkb1-wt cells. However, the more sensitive analysis by flow cytometry confirmed higher rates of baseline DNA damage in Lkb1-null than Lkb1-wt cells (2.57%, 8.53%, 5.08% vs. 1.80%, 1.71%, 2.01%) (FIG. 6C, upper panels). Further 3 h treatment with AZD7762 results in more cells having DNA DSBs in Lkb1-null cells than Lkb1-wt cells (7.76%, 17.10%, 10.30% vs. 3.06%, 4.42%, 2.88%) (FIG. 6C, lower panels). These resultant levels of DNA damage, especially DNA DSBs, may cause the observed synthetic lethal effect of Chek1 knockdown in Lkb1-null cells.

### Example 11

### Combination Treatment Diminishes the Size of Lkb1-Null Tumors

The change in uptake of ¹⁸F-fluoro-2-deoxy-glucose (18-FDG) estimated by positron emission tomography (PET) has been demonstrated to be a biomarker for treatment response (Chen et al., 2012; Vansteenkiste et al., 1999). This method was used to examine the therapeutic efficacy of CHEK1 inhibition on Lkb1-null tumors in vivo. A total of 9 mice (3 Kras/p53, 3 Kras/Lkb1, and 3 Kras/p53/Lkb1) with lung cancer were imaged before treatment and each mouse showed at least one hypermetabolic tumor nodule (FIG. 7A, arrowhead). After receiving 2 doses of AZD7622, the mice were imaged again, revealing notable, genotype-specific differences in the 18-FDG uptake of their tumors (FIG. 7A). Specifically, short term AZD7622 treatment was most effective in reducing 18-FDG uptake in Kras/p53/Lkb1 tumors, followed by little or no response in Kras/Lkb1 and Kras/p53 tumors, respectively. Of the three tumor genotypes, only triple-mutant tumors showed an overall decrease in hypermetabolic activity post-treatment. These results are consistent with our in vitro data that cell lines derived from Kras/p53/Lkb1 tumors are most responsive to AZD7622 treatment.

As CHEK1 inhibitors have been used clinically to enhance the effect of radiotherapy or genotoxic drugs, an in vitro study was performed to search for suitable combination treatments with CHEK1 inhibitor AZD7762. Gemcitabine (a deoxycytidine-analogue) was identified to be moderately synergistic with AZD7762 in the treatment of Lkb1-null cells (data not shown). To test the clinical applicability of this observation to KRAS-driven, LKB1-deficient human lung cancer, xenograft studies were performed using two LKB1-deficient human NSCLC cell lines, A549 and H2122, in comparison with Lkb1-null murine lines (t2, t4, and t5). Synergistic treatment effects with AZD7762 and gemcitabine combination in both human and mouse xenografts was observed (FIGS. 7B and 7C). These data provide an additional support for potential clinical application of this combination for the subset of KRAS-driven lung cancer patients with concurrent LKB1 loss.

TABLE 1
Top 100 shRNAs

| | | | | | # | # | # | # |
|---|---|---|---|---|---|---|---|---|
| | | # | | Hairpin | Hairpins | Hairpins | Hairpins | Hairpins |
| Hairpin | Gene | Hairpins | Hairpin ranks | rank | 500 | 1000 | 5000 | 10000 |
| TRCN0000042724 | Tsc2 | 3 | 24573, 26660, 1 | 1 | 1 | 1 | 1 | 1 |
| TRCN0000065525 | Kcnk13 | 4 | 17671, 26655, 4330, 2 | 2 | 1 | 1 | 2 | 2 |
| TRCN0000042598 | Cdkn2b | 4 | 4883, 11284, 3588, 3 | 3 | 1 | 1 | 3 | 3 |
| TRCN0000012089 | Nfib | 5 | 5346, 27131, 32330, 33927, 4 | 4 | 1 | 1 | 1 | 2 |
| TRCN0000104023 | Trmt2b | 5 | 13091, 18857, 30595, 32469, 5 | 5 | 1 | 1 | 1 | 1 |
| TRCN0000095162 | Pcgf5 | 5 | 18901, 6948, 19738, 6, 22384 | 6 | 1 | 1 | 1 | 2 |
| TRCN0000025401 | Cmpk1 | 4 | 7805, 21438, 33733, 7 | 7 | 1 | 1 | 1 | 2 |
| TRCN0000095044 | E430018J23Rik | 2 | 8, 32974 | 8 | 1 | 1 | 1 | 1 |
| TRCN0000077063 | Ifng | 5 | 26405, 6934, 29781, 33241, 9 | 9 | 1 | 1 | 1 | 2 |
| TRCN0000066676 | Cd83 | 5 | 20018, 25899, 9010, 9232, 10 | 10 | 1 | 1 | 1 | 3 |
| TRCN0000087343 | LOC436441 | 3 | 24616, 6640, 11 | 11 | 1 | 1 | 1 | 2 |
| TRCN0000091186 | Krt26 | 4 | 28196, 30868, 12, 14882 | 12 | 1 | 1 | 1 | 1 |
| TRCN0000022971 | LOC329302 | 3 | 18575, 29957, 13 | 13 | 1 | 1 | 1 | 1 |
| TRCN0000105531 | Mup21 | 5 | 5137, 16197, 23302, 14, 33659 | 14 | 1 | 1 | 1 | 2 |
| TRCN0000098846 | Oplah | 5 | 19930, 24932, 25915, 26711, 15 | 15 | 1 | 1 | 1 | 1 |
| TRCN0000094444 | Pcdhb15 | 5 | 32882, 9262, 16, 8485, 1670 | 16 | 1 | 1 | 2 | 4 |
| TRCN0000097345 | Hsh2d | 5 | 20171, 42, 17, 3469, 1308 | 17 | 2 | 2 | 4 | 4 |
| TRCN0000092488 | Pdcl3 | 4 | 9302, 251, 30142, 18 | 18 | 2 | 2 | 2 | 3 |
| TRCN0000066487 | Tnfrsf8 | 5 | 19, 9783, 280, 5877, 5922 | 19 | 2 | 2 | 2 | 5 |
| TRCN0000103560 | Foxj1 | 5 | 12270, 9392, 25640, 24869, 20 | 20 | 1 | 1 | 1 | 2 |
| TRCN0000042546 | Rb1 | 6 | 9047, 7122, 2696, 2236, 21, 971 | 21 | 1 | 2 | 4 | 6 |
| TRCN0000012563 | Bmi1 | 4 | 10014, 1 [symbol] 712, 11148, 22 | 22 | 1 | 1 | 1 | 1 |
| TRCN0000070416 | Hoxd12 | 5 | 17899, 23, 26599, 21756, 29892 | 23 | 1 | 1 | 1 | 1 |
| TRCN0000094702 | Pcdha2 | 4 | 27780, 24, 30160, 32030 | 24 | 1 | 1 | 1 | 1 |
| TRCN0000088149 | Cdc16 | 5 | 16735, 31994, 7452, 31454, 25 | 25 | 1 | 1 | 1 | 2 |
| TRCN0000025435 | Dgki | 5 | 354, 31017, 2028, 26, 34166 | 26 | 2 | 2 | 3 | 3 |
| TRCN0000103628 | Hcls1 | 4 | 9297, 15681, 30131, 27 | 27 | 1 | 1 | 1 | 2 |
| TRCN0000041007 | Pja2 | 3 | 28, 29 [symbol] 26, 3026 | 28 | 1 | 1 | 2 | 2 |
| TRCN0000077588 | Rasgrf1 | 2 | 18016, 29 | 29 | 1 | 1 | 1 | 1 |
| TRCN0000091867 | Arp [symbol] 1 | 4 | 15384, 29362, 29332, 30 | 30 | 1 | 1 | 1 | 1 |
| TRCN0000022987 | Cdk13 | 5 | 5591, 4242, 22081, 31, 1173 | 31 | 1 | 1 | 3 | 4 |
| TRCN0000055266 | Hnmpa1 | 5 | 486, 31915, 33099, 24020, 32 | 32 | 2 | 2 | 2 | 2 |
| TRCN0000101337 | Fabp9 | 4 | 23270, 416, 32119, 33 | 33 | 2 | 2 | 2 | 2 |
| TRCN0000092233 | LOC432889 | 5 | 11274, 9799, 20792, 34, 30456 | 34 | 1 | 1 | 1 | 2 |
| TRCN0000091224 | Grid2ip | 4 | 14756, 16364, 16110, 35 | 35 | 1 | 1 | 1 | 1 |
| TRCN0000079507 | Slc35d1 | 4 | 20923, 9933, 3700, 36 | 36 | 1 | 1 | 2 | 3 |
| TRCN0000023870 | Riok3 | 4 | 7509, 30818, 33652, 37 | 37 | 1 | 1 | 1 | 2 |
| TRCN0000099729 | Zfp292 | 5 | 38, 30015, 4872, 1667, 2634 | 38 | 1 | 1 | 4 | 4 |
| TRCN0000089883 | Tuba3b | 4 | 12199, 16436, 22234, 39 | 39 | 1 | 1 | 1 | 1 |
| TRCN0000071307 | Terf2 | 5 | 27110, 27310, 40, 15699, 338 | 40 | 2 | 2 | 2 | 2 |
| TRCN0000097348 | Hsh2d | 5 | 20171, 42, 17, 3469, 1308 | 41 | 2 | 2 | 4 | 4 |
| TRCN0000030910 | Usp54 | 9 | 9399, 4804, 23756, 22698, 27158, | 42 | 1 | 1 | 3 | 4 |
| | | | 29817, 43, 32216, 3131 | | | | | |
| TRCN0000071317 | Adar | 4 | 25676, 44, 31960, 2301 | 43 | 1 | 1 | 2 | 2 |
| TRCN0000086359 | Zfat | 5 | 34131, 1445, 208, 34245, 45 | 44 | 2 | 2 | 3 | 3 |
| TRCN0000030435 | Olfr125 [symbol] | 4 | 7266, 2389, 18465, 46 | 45 | 1 | 1 | 2 | 3 |
| TRCN0000041829 | Pdhb | 4 | 12474, 5193, 14721, 47 | 46 | 1 | 1 | 1 | 2 |
| TRCN0000040972 | Zswim2 | 3 | 27141, 48, 33882 | 47 | 1 | 1 | 1 | 1 |
| TRCN0000023516 | Ntrk3 | 2 | 8462, 49 | 48 | 1 | 1 | 1 | 2 |
| TRCN0000091007 | 4732456N10Rik | 4 | 21340, 24037, 17584, 50 | 49 | 1 | 1 | 1 | 1 |
| TRCN0000081987 | Nfe2 | 5 | 27993, 31616, 28479, 51, 1154 | 50 | 1 | 1 | 2 | 2 |
| TRCN0000094496 | Pcdhb12 | 5 | 20116, 23619, 12824, 52, 13293 | 51 | 1 | 1 | 1 | 1 |
| TRCN0000040725 | Rnf146 | 5 | 1793, 3390, 3763, 6435, 53 | 52 | 1 | 1 | 4 | 5 |
| TRCN0000066222 | Ifna2 | 5 | 10320, 26687, 26956, 5617, 55 | 53 | 1 | 1 | 1 | 2 |
| TRCN0000094712 | Tspan33 | 2 | 31106, 56 | 54 | 1 | 1 | 1 | 1 |
| TRCN0000012739 | Nedd8 | 3 | 9680, 57, 6779 | 55 | 1 | 1 | 1 | 3 |
| TRCN0000094729 | Cdh4 | 5 | 11343, 4937, 29012, 58, 33201 | 56 | 1 | 1 | 2 | 2 |
| TRCN0000012006 | Fstl1 | 5 | 7250, 32604, 59, 33219, 34170 | 57 | 1 | 1 | 1 | 2 |
| TRCN0000102470 | Pspc1 | 2 | 29066, 60 | 58 | 1 | 1 | 1 | 1 |
| TRCN0000086895 | H60 [symbol] | 5 | 21073, 19729, 27589, 81, 2481 | 59 | 1 | 1 | 2 | 2 |
| TRCN00000243 [symbol] | Tssk1 | 4 | 7660, 24712, 32333, 62 | 60 | 1 | 1 | 1 | 2 |
| TRCN0000040745 | Znrf1 | 3 | 21577, 33153, 63 | 61 | 1 | 1 | 1 | 1 |
| TRCN0000040629 | Usp24 | 4 | 22191, 17559, 19572, 64 | 62 | 1 | 1 | 1 | 1 |
| TRCN0000096130 | Zfp85-rs1 | 5 | 1114, 65, 1628, 25701, 31608 | 63 | 1 | 1 | 3 | 3 |
| TRCN0000102700 | Rhoh | 5 | 8007, 18501, 23532, 30830, 66 | 64 | 1 | 1 | 1 | 2 |
| TRCN0000025391 | LOC381757 | 4 | 18804, 20444, 67, 1552 | 65 | 1 | 1 | 2 | 2 |
| TRCN0000091053 | Kiss1 | 4 | 12472, 68, 28452, 21892 | 66 | 1 | 1 | 1 | 1 |
| TRCN0000022580 | Map3K7 | 4 | 20793, 69, 29059, 32638 | 67 | 1 | 1 | 1 | 1 |
| TRCN0000088943 | Mgp | 4 | 4736, 2776, 70, 28075 | 68 | 1 | 1 | 3 | 3 |
| TRCN0000099162 | Cbr4 | 4 | 22070, 4013, 21153, 71 | 69 | 1 | 1 | 2 | 2 |
| TRCN0000065794 | Clec2g | 4 | 24674, 72, 32680, 1459 | 70 | 1 | 1 | 2 | 2 |
| TRCN0000012128 | Nfe2l2 | 9 | 11732, 13344, 1687, 24782, 28676, | 71 | 1 | 1 | 3 | 3 |
| | | | 1684, 26365, 31232, 73 | | | | | |
| TRCN0000070819 | Zeb1 | 5 | 13623, 12678, 29498, 3511, 74 | 72 | 1 | 1 | 2 | 2 |
| TRCN0000087484 | Gm6194 | 5 | 10271, 17753, 7310, 75, 29200 | 73 | 1 | 1 | 1 | 2 |
| TRCN0000095311 | Ppargc1a | 5 | 13882, 337, 29127, 76, 28575 | 74 | 2 | 2 | 2 | 2 |
| TRCN0000093764 | [symbol] gcg | 5 | 16838, 77, 19225, 29226, 21994 | 75 | 1 | 1 | 1 | 1 |
| TRCN0000092795 | Sp100 | 5 | 15141, 29499, 78, 6450, 22526 | 76 | 1 | 1 | 1 | 2 |
| TRCN0000079320 | Slc15a2 | 4 | 11166, 29505, 31633, 79 | 77 | 1 | 1 | 1 | 1 |
| TRCN0000104628 | Gnptg | 5 | 2376, 24944, 31636, 701, 80 | 78 | 1 | 2 | 3 | 3 |
| TRCN0000095 [symbol] 9 [symbol] | 2610008E11Rik | 5 | 22476, 29796, 4782, 16465, 81 | 79 | 1 | 1 | 2 | 2 |
| TRCN0000023639 | Fert2 | 5 | 82, 18572, 33682, 33372, 20045 | 80 | 1 | 1 | 1 | 1 |
| TRCN0000041103 | 1700045l19Rik | 4 | 12167, 14828, 83, 33957 | 81 | 1 | 1 | 1 | 1 |
| TRCN0000022612 | Araf | 6 | 13139, 24458, 6810, 3106, 32936, 84 | 82 | 1 | 1 | 2 | 3 |
| TRCN0000081995 | Dmbx1 | 3 | 25725, 23980, 85 | 83 | 1 | 1 | 1 | 1 |
| TRCN0000022508 | Tesk2 | 4 | 6591, 32055, 86, 30300 | 84 | 1 | 1 | 1 | 2 |
| TRCN0000102131 | Sec14l4 | 5 | 6082, 31752, 27834, 30605, 87 | 85 | 1 | 1 | 1 | 2 |
| TRCN0000086619 | Zkscan5 | 5 | 20736, 16621, 751, 88, 4807 | 86 | 1 | 2 | 3 | 3 |
| TRCN0000079267 | Slco4c1 | 3 | 8400, 30571, 89 | 87 | 1 | 1 | 1 | 2 |
| TRCN0000094352 | Pcdha4 | 3 | 19855, 21576, 90 | 88 | 1 | 1 | 1 | 1 |
| TRCN0000103535 | Kdm4b | 5 | 14611, 7574, 30612, 11437, 91 | 89 | 1 | 1 | 1 | 2 |
| TRCN0000024857 | Prkar2a | 5 | 14529, 27449, 27283, 92, 33924 | 90 | 1 | 1 | 1 | 1 |
| TRCN0000012725 | Crebbp | 5 | 8296, 93, 2265, 33276, 7201 | 91 | 1 | 1 | 2 | 4 |
| TRCN0000081636 | Ebf4 | 4 | 12366, 15177, 33004, 94 | 92 | 1 | 1 | 1 | 1 |
| TRCN0000067762 | Il13ra1 | 5 | 19278, 11748, 2868, 95, 30454 | 93 | 1 | 1 | 2 | 2 |
| TRCN0000071203 | Ep300 | 5 | 9270, 26544, 96, 3200, 211 | 94 | 2 | 2 | 3 | 4 |
| TRCN0000097295 | Dvl3 | 3 | 23191, 24501, 97 | 95 | 1 | 1 | 1 | 1 |
| TRCN0000028991 | Pten | 4 | 5080, 98, 7407, 481 | 96 | 2 | 2 | 2 | 4 |
| TRCN0000096474 | Ncor1 | 5 | 18426, 11856, 6890, 521, 99 | 97 | 1 | 2 | 2 | 3 |
| TRCN0000024616 | Dtymk | 4 | 5740, 22800, 21302, 100 | 98 | 1 | 1 | 1 | 2 |
| TRCN0000037275 | Rnf31 | 4 | 4505, 17491, 101, 32573 | 99 | 1 | 1 | 2 | 2 |
| TRCN0000030486 | Ren1 | 4 | 9911, 3960, 102, 7590 | 100 | 1 | 1 | 2 | 4 |

[symbol]

indicates data missing or illegible when filed

TABLE 2
Top 200 genes by KS

| | | | | | | | | # Hairpins | # Hairpins | # Hairpins | # Hairpins |
|---|---|---|---|---|---|---|---|---|---|---|---|
| Gene | Hairpins | # Hairpins | Hairpin ranks | NES | Gene rank | p-value | p-value rank | 500 | 1000 | 5000 | 10000 |
| Grid2ip | TRCN0000091223, | 4 | 14756, 16364, 16110, 35 | 1.64 | 10 | 0.00007 | 1 | 1 | 1 | 1 | 1 |
| Tsc2 | TRCN0000042727, | 3 | 24573, 26660, 1 | 1.56 | 65 | 0.00016 | 2 | 1 | 1 | 1 | 1 |
| Pcgf5 | TRCN0000095160, | 5 | 18901, 6948, 19738, 6, 22384 | 1.69 | 1 | 0.00034 | 3 | 1 | 1 | 1 | 2 |
| Kcnk13 | TRCN0000065526, | 4 | 17671, 26655, 4330, 2 | 1.63 | 16 | 0.00061 | 4 | 1 | 1 | 2 | 2 |
| Bmi1 | TRCN0000012564, | 4 | 10014, 19712, 11148, 22 | 1.63 | 17 | 0.0007 | 5 | 1 | 1 | 1 | 1 |
| Rasgrt1 | TRCN0000077591, | 2 | 18016, 29 | 1.46 | 239 | 0.00093 | 6 | 1 | 1 | 1 | 1 |
| Cd83 | TRCN0000066675, | 5 | 20018, 25899, 9010, 9232, 10 | 1.68 | 2 | 0.00095 | 7 | 1 | 1 | 1 | 3 |
| Cdkn2b | TRCN0000042599, | 4 | 4883, 11284, 3588, 3 | 1.62 | 19 | 0.0011 | 8 | 1 | 1 | 3 | 3 |
| Slc7a10 | TRCN0000079468, | 5 | 12876, 13533, 106, 10453, 14857 | 1.67 | 3 | 0.0012 | 9 | 1 | 1 | 1 | 1 |
| Tuba3b | TRCN0000089884, | 4 | 12199, 16436, 22234, 39 | 1.62 | 22 | 0.0013 | 10 | 1 | 1 | 1 | 1 |
| Trmt2b | TRCN0000104021, | 5 | 13091, 18857, 30596, 32469, 5 | 1.67 | 4 | 0.0014 | 11 | 1 | 1 | 1 | 1 |
| Ptprg | TRCN0000029952, | 3 | 14299, 14563, 247 | 1.55 | 80 | 0.0015 | 12 | 1 | 1 | 1 | 1 |
| Foxj1 | TRCN0000103562, | 5 | 12270, 9392, 25640, 24869, 20 | 1.66 | 5 | 0.0017 | 13 | 1 | 1 | 1 | 2 |
| Tnfrsf8 | TRCN0000066487, | 5 | 19, 9783, 280, 5877, 5922 | 1.66 | 6 | 0.002 | 14 | 2 | 2 | 2 | 5 |
| Pcdhb12 | TRCN0000094497, | 5 | 20116, 23619, 12824, 52, 13293 | 1.66 | 7 | 0.0021 | 15 | 1 | 1 | 1 | 1 |
| LOC438441 | TRCN0000087344, | 3 | 24616, 8640, 11 | 1.54 | 87 | 0.0022 | 16 | 1 | 1 | 1 | 2 |
| Oplah | TRCN0000098847, | 5 | 19930, 24932, 25915, 28711, 15 | 1.55 | 8 | 0.0025 | 17 | 1 | 1 | 1 | 1 |
| LOC433793 | TRCN0000080996, | 3 | 12266, 13448, 159 | 1.54 | 90 | 0.0027 | 18 | 1 | 1 | 1 | 1 |
| Cmpk1 | TRCN0000025402, | 4 | 7805, 21438, 33733, 7 | 1.6 | 28 | 0.0029 | 19 | 1 | 1 | 1 | 2 |
| Pdcl3 | TRCN0000092490, | 4 | 9302, 251, 30142, 18 | 1.6 | 29 | 0.003 | 20 | 2 | 2 | 2 | 3 |
| Usp24 | TRCN0000040631, | 4 | 22191, 17559, 19572, 64 | 1.6 | 31 | 0.0032 | 21 | 1 | 1 | 1 | 1 |
| Pdhb | TRCN0000041832, | 4 | 12474, 5193, 14721, 47 | 1.6 | 32 | 0.0033 | 22 | 1 | 1 | 1 | 2 |
| Tfpi2 | TRCN0000080031, | 5 | 12576, 285, 6502, 558, 254 | 1.64 | 9 | 0.0034 | 23 | 2 | 3 | 3 | 4 |
| LOC329302 | TRCN0000022973, | 3 | 18575, 29957, 13 | 1.53 | 95 | 0.0034 | 24 | 1 | 1 | 1 | 1 |
| 4732456N10Rik | TRCN0000091004, | 4 | 21340, 24037, 17584, 50 | 1.6 | 35 | 0.0037 | 25 | 1 | 1 | 1 | 1 |
| Slc25a28 | TRCN0000069611, | 3 | 13954, 15676, 440 | 1.53 | 97 | 0.0037 | 26 | 1 | 1 | 1 | 1 |
| Ntrk3 | TRCN0000023518, | 2 | 8462, 49 | 1.45 | 279 | 0.0037 | 27 | 1 | 1 | 1 | 2 |
| Nfib | TRCN0000012091, | 5 | 5346, 27131, 32330, 33927, 4 | 1.64 | 11 | 0.0038 | 28 | 1 | 1 | 1 | 2 |
| Ncor1 | TRCN0000096475, | 5 | 18426, 11856, 6890, 521, 99 | 1.54 | 12 | 0.0039 | 29 | 1 | 2 | 2 | 3 |
| Krt26 | TRCN0000091184, | 4 | 28196, 30868, 12, 14882 | 1.59 | 38 | 0.0039 | 30 | 1 | 1 | 1 | 1 |
| Calm4 | TRCN0000104619, | 3 | 13985, 10687, 181 | 1.53 | 99 | 0.0039 | 31 | 1 | 1 | 1 | 1 |
| Hcls1 | TRCN0000103626, | 4 | 9297, 15681, 30131, 27 | 1.59 | 40 | 0.0041 | 32 | 1 | 1 | 1 | 2 |
| Hist1h1a | TRCN0000097053, | 3 | 16825, 11658, 196 | 1.53 | 100 | 0.0041 | 33 | 1 | 1 | 1 | 1 |
| Terf2 | TRCN0000071304, | 5 | 27110, 27310, 40, 15699, 338 | 1.63 | 14 | 0.0042 | 34 | 2 | 2 | 2 | 2 |
| Hoxd12 | TRCN0000070415, | 5 | 17899, 23, 26599, 21756, 29892 | 1.63 | 15 | 0.0043 | 35 | 1 | 1 | 1 | 1 |
| Slc36a3 | TRCN0000068343, | 2 | 11399, 137 | 1.44 | 288 | 0.005 | 36 | 1 | 1 | 1 | 1 |
| LOC432889 | TRCN0000092237, | 5 | 11274, 9799, 20792, 34, 30456 | 1.62 | 18 | 0.0051 | 37 | 1 | 1 | 1 | 2 |
| Gmeb1 | TRCN0000081631, | 3 | 15765, 496, 877 | 1.52 | 113 | 0.0051 | 38 | 1 | 2 | 2 | 2 |
| Nedd8 | TRCN0000012742, | 3 | 9680, 57, 6779 | 1.52 | 115 | 0.0052 | 39 | 1 | 1 | 1 | 3 |
| Hsh2d | TRCN0000097347, | 5 | 20171, 42, 17, 3469, 1308 | 1.62 | 20 | 0.0054 | 40 | 2 | 2 | 4 | 4 |
| Flnb | TRCN0000091374, | 5 | 14978, 22564, 18232, 16023, 189 | 1.62 | 21 | 0.0055 | 41 | 1 | 1 | 1 | 1 |
| Ifng | TRCN0000077057, | 5 | 26405, 6934, 29781, 33241, 9 | 1.62 | 23 | 0.0058 | 42 | 1 | 1 | 1 | 2 |
| Cd84 | TRCN0000066282, | 4 | 9400, 15105, 788, 425 | 1.58 | 50 | 0.0059 | 43 | 1 | 2 | 2 | 3 |
| Ift57 | TRCN0000100246, | 4 | 11465, 16596, 782, 749 | 1.58 | 51 | 0.0061 | 44 | 0 | 2 | 2 | 2 |
| Pcdha4 | TRCN0000094349, | 3 | 19855, 21576, 90 | 1.52 | 121 | 0.0062 | 45 | 1 | 1 | 1 | 1 |
| Atp1b1 | TRCN0000054955, | 3 | 14656, 1020, 435 | 1.52 | 122 | 0.0063 | 46 | 1 | 1 | 2 | 2 |
| Ikbke | TRCN0000026726, | 4 | 8965, 8075, 17662, 112 | 1.58 | 53 | 0.0064 | 47 | 1 | 1 | 1 | 3 |
| Actn2 | TRCN0000072024, | 4 | 11458, 15109, 6095, 111 | 1.57 | 54 | 0.0065 | 48 | 1 | 1 | 1 | 2 |
| Mup21 | TRCN0000105533, | 5 | 5137, 16197, 23302, 14, 33659 | 1.61 | 24 | 0.0067 | 49 | 1 | 1 | 1 | 2 |
| Rasgrf2 | TRCN0000077593, | 5 | 10322, 18435, 342, 15164, 13168 | 1.61 | 25 | 0.0067 | 50 | 1 | 1 | 1 | 1 |
| E430018J23Rik | TRCN0000095044, | 2 | 8, 32874 | 1.44 | 309 | 0.0072 | 51 | 1 | 1 | 1 | 1 |
| Slc35d1 | TRCN0000079506, | 4 | 20923, 9933, 3700, 38 | 1.57 | 62 | 0.0077 | 52 | 1 | 1 | 2 | 3 |
| Ppp2r4 | TRCN0000077223, | 5 | 27244, 7023, 113, 16308, 436 | 1.6 | 27 | 0.0078 | 53 | 2 | 2 | 2 | 3 |
| Bnip3l | TRCN0000009730, | 3 | 11846, 15323, 517 | 1.51 | 134 | 0.008 | 54 | 0 | 1 | 1 | 1 |
| Cxcl14 | TRCN0000065371, | 5 | 11935, 14043, 12433, 18415, 391 | 1.6 | 30 | 0.008 | 55 | 1 | 1 | 1 | 1 |
| Sult1d1 | TRCN0000103272, | 5 | 17571, 17805, 16728, 152, 1642 | 1.6 | 33 | 0.0082 | 56 | 1 | 1 | 2 | 2 |
| Trhr | TRCN0000027853, | 3 | 19412, 19006, 184 | 1.51 | 137 | 0.0082 | 57 | 1 | 1 | 1 | 1 |
| Arhgap8 | TRCN0000097305, | 5 | 15079, 264, 27349, 7684, 302 | 1.6 | 34 | 0.0085 | 58 | 2 | 2 | 2 | 3 |
| Zkscan5 | TRCN0000086622, | 5 | 20736, 16621, 751, 88, 4807 | 1.59 | 36 | 0.0087 | 59 | 1 | 2 | 3 | 3 |
| Sh2d1b1 | TRCN0000081259, | 5 | 15974, 6119, 303, 125, 1572 | 1.59 | 37 | 0.0087 | 60 | 2 | 2 | 3 | 4 |
| Arpm1 | TRCN0000091864, | 4 | 15384, 29362, 29332, 30 | 1.56 | 68 | 0.0088 | 61 | 1 | 1 | 1 | 1 |
| Gm13232 | TRCN0000092939, | 4 | 17528, 624, 14367, 16798 | 1.56 | 69 | 0.0089 | 62 | 0 | 1 | 1 | 1 |
| Mark1 | TRCN0000024171, | 4 | 12821, 13331, 16244, 626 | 1.56 | 71 | 0.009 | 63 | 0 | 1 | 1 | 1 |
| Cdc16 | TRCN0000088152, | 5 | 18735, 31994, 7452, 31454, 25 | 1.59 | 39 | 0.009 | 64 | 1 | 1 | 1 | 2 |
| Phlda2 | TRCN0000055085, | 5 | 11265, 17575, 987, 19321, 482 | 1.59 | 41 | 0.0092 | 65 | 1 | 2 | 2 | 2 |
| Myt1l | TRCN0000012110, | 3 | 14205, 17081, 649 | 1.5 | 148 | 0.0093 | 66 | 0 | 1 | 1 | 1 |
| Ppargc1a | TRCN0000095310, | 5 | 13882, 337, 29127, 78, 28575 | 1.59 | 42 | 0.0095 | 67 | 2 | 2 | 2 | 2 |
| Fabp9 | TRCN0000101 [symbol] 36, | 4 | 23270, 416, 32119, 33 | 1.56 | 72 | 0.0095 | 68 | 2 | 2 | 2 | 2 |
| Nqo1 | TRCN0000041865, | 5 | 11346, 15157, 17204, 6848, 230 | 1.59 | 43 | 0.0096 | 69 | 1 | 1 | 1 | 2 |
| Kiss1 | TRCN0000091055, | 4 | 12472, 68, 28452, 21892 | 1.55 | 73 | 0.0097 | 70 | 1 | 1 | 1 | 1 |
| Slc35a1 | TRCN0000079790, | 3 | 17946, 8288, 183 | 1.5 | 149 | 0.0097 | 71 | 1 | 1 | 1 | 2 |
| Pgc | TRCN0000030478, | 2 | 11712, 311 | 1.43 | 330 | 0.0098 | 72 | 1 | 1 | 1 | 1 |
| Ugcg | TRCN0000093767, | 5 | 16838, 77, 19225, 29226, 21994 | 1.59 | 44 | 0.0099 | 73 | 1 | 1 | 1 | 1 |
| Krt78 | TRCN0000091189, | 5 | 5226, 19437, 135, 24499, 355 | 1.59 | 45 | 0.0099 | 74 | 2 | 2 | 2 | 3 |
| Pten | TRGN0000028993, | 4 | 5080, 98, 7407, 481 | 1.55 | 75 | 0.01 | 75 | 2 | 2 | 2 | 4 |
| Gm6194 | TRCN0000087486, | 5 | 10271, 17753, 7310, 75, 29200 | 1.58 | 46 | 0.01 | 76 | 1 | 1 | 1 | 2 |
| Rab11a | TRCN0000100341, | 3 | 19345, 8384, 161 | 1.5 | 152 | 0.01 | 77 | 1 | 1 | 1 | 2 |
| Scamp3 | TRCN0000105364, | 4 | 14146, 225, 15537, 1911 | 1.55 | 78 | 0.011 | 78 | 1 | 1 | 2 | 2 |
| Ptges3 | TRCN0000071289, | 5 | 8150, 2092, 1402, 738, 226 | 1.58 | 47 | 0.011 | 79 | 1 | 2 | 4 | 5 |
| Gm5689 | TRCN0000092057, | 2 | 587, 16383 | 1.43 | 341 | 0.011 | 80 | 0 | 1 | 1 | 1 |
| Cd38 | TRCN0000068230, | 5 | 15467, 201, 2617, 894, 1456 | 1.58 | 48 | 0.011 | 81 | 1 | 2 | 4 | 4 |
| LOC381757 | TRCN0000025392, | 4 | 18804, 20444, 87, 1552 | 1.55 | 81 | 0.011 | 82 | 1 | 1 | 2 | 2 |
| Cd22 | TRCN0000067947, | 5 | 17599, 17396, 2061, 14799, 232 | 1.58 | 49 | 0.011 | 83 | 1 | 1 | 2 | 2 |
| Zfp955a | TRCN0000104861, | 3 | 13945, 235, 1470 | 1.49 | 162 | 0.011 | 84 | 1 | 1 | 2 | 2 |
| Pcdhb15 | TRCN0000094447, | 5 | 32582, 9262, 16, 8485, 1670 | 1.58 | 52 | 0.012 | 85 | 1 | 1 | 2 | 4 |
| Rb1 | TRCN0000055378, | 6 | 9047, 7122, 2696, 2236, 21, 971 | 1.6 | 26 | 0.012 | 86 | 1 | 2 | 4 | 6 |
| | TRCN0000055380, | | | | | | | | | | |
| Uba1 | TRCN0000012744, | 4 | 17864, 200, 17663, 23474 | 1.54 | 84 | 0.012 | 87 | 1 | 1 | 1 | 1 |
| Cd160 | TRCN0000066917, | 4 | 15779, 16462, 941, 1767 | 1.54 | 86 | 0.012 | 88 | 0 | 1 | 2 | 2 |
| Cx3cr1 | TRCN0000026669, | 2 | 995, 756 | 1.42 | 359 | 0.012 | 89 | 0 | 2 | 2 | 2 |
| Zfp119a | TRCN0000085401, | 5 | 3213, 144, 1232, 939, 287 | 1.57 | 55 | 0.012 | 90 | 2 | 3 | 5 | 5 |
| Zfp354c | TRCN0000082343, | 3 | 15588, 1606, 204 | 1.49 | 165 | 0.012 | 91 | 1 | 1 | 2 | 2 |
| Ankrd7 | TRCN0000103882, | 5 | 24725, 7505, 329, 19140, 452 | 1.57 | 56 | 0.012 | 92 | 2 | 2 | 2 | 3 |
| Fars2 | TRCN0000076317, | 2 | 18676, 332 | 1.42 | 361 | 0.012 | 93 | 1 | 1 | 1 | 1 |
| Ep300 | TRCN0000071205, | 5 | 9270, 26544, 96, 3200, 211 | 1.57 | 57 | 0.012 | 94 | 2 | 2 | 3 | 4 |
| Lfb4r2 | TRCN0000027908, | 2 | 10933, 339 | 1.42 | 363 | 0.013 | 95 | 1 | 1 | 1 | 1 |
| Itga7 | TRCN0000066189, | 3 | 12449, 18211, 472 | 1.49 | 167 | 0.013 | 96 | 1 | 1 | 1 | 1 |
| Cdk13 | TRCN0000022984, | 5 | 5591, 4242, 22081, 31, 1173 | 1.57 | 59 | 0.013 | 97 | 1 | 1 | 3 | 4 |
| Ifna2 | TRCN0000066218, | 5 | 10320, 26687, 28958, 5617, 55 | 1.57 | 58 | 0.013 | 98 | 1 | 1 | 1 | 2 |
| Slc25a30 | TRCN0000068748, | 4 | 14527, 15124, 1453, 2141 | 1.54 | 91 | 0.013 | 99 | 0 | 0 | 2 | 2 |
| Chek1 | TRCN0000012648, | 5 | 18074, 20941, 18114, 12257, 327 | 1.57 | 61 | 0.013 | 100 | 1 | 1 | 1 | 1 |
| Ly6a | TRCN0000100120, | 4 | 6348, 1478, 855, 162 | 1.54 | 92 | 0.013 | 101 | 1 | 2 | 3 | 4 |
| Pja1 | TRCN0000098562, | 5 | 14694, 5037, 143, 18688, 19267 | 1.57 | 63 | 0.013 | 102 | 1 | 1 | 1 | 2 |
| Atrx | TRCN0000081909, | 4 | 10815, 21625, 13720, 304 | 1.53 | 93 | 0.013 | 103 | 1 | 1 | 1 | 1 |
| Icos | TRCN0000066859, | 5 | 17479, 5865, 1874, 658, 224 | 1.56 | 64 | 0.014 | 104 | 1 | 2 | 3 | 4 |
| Rhoh | TRCN0000102701, | 5 | 8007, 18501, 23532, 30830, 88 | 1.56 | 88 | 0.014 | 105 | 1 | 1 | 1 | 2 |
| Dvl3 | TRCN0000097297, | 3 | 23191, 24501, 97 | 1.48 | 180 | 0.014 | 106 | 1 | 1 | 1 | 1 |
| Sp100 | TRCN0000092797, | 5 | 15141, 29499, 78, 6450, 22526 | 1.56 | 67 | 0.014 | 107 | 1 | 1 | 1 | 2 |
| Kdm4b | TRCN0000103537, | 5 | 14611, 7574, 30612, 11437, 91 | 1.56 | 70 | 0.014 | 108 | 1 | 1 | 1 | 2 |
| Pcdha2 | TRCN0000094699, | 4 | 27780, 24, 30160, 32030 | 1.53 | 98 | 0.015 | 109 | 1 | 1 | 1 | 1 |
| Dtymk | TRCN0000024618, | 4 | 5740, 22800, 21302, 100 | 1.53 | 103 | 0.015 | 110 | I | 1 | 1 | 2 |
| Dmbx1 | TRCN0000081994, | 3 | 25726, 23980, 85 | 1.48 | 188 | 0.015 | 111 | 1 | 1 | 1 | 1 |
| Med10 | TRCN0000082032, | 4 | 26707, 188, 27196, 271 | 1.53 | 105 | 0.015 | 112 | 2 | 2 | 2 | 2 |
| Cbr4 | TRCN0000099160, | 4 | 22070, 4013, 21153, 71 | 1.53 | 106 | 0.015 | 113 | 1 | 1 | 2 | 2 |
| Dok2 | TRCN0000077432, | 5 | 14484, 21493, 1294, 18506, 1073 | 1.55 | 74 | 0.015 | 114 | 0 | 0 | 2 | 2 |
| Usp35 | TRCN0000092451, | 4 | 13597, 9316, 5631, 156 | 1.53 | 107 | 0.015 | 115 | 1 | 1 | 1 | 3 |
| Gm7358 | TRCN0000024085, | 4 | 20504, 17853, 13539, 451 | 1.52 | 109 | 0.016 | 116 | 1 | 1 | 1 | 1 |
| Stx18 | TRCN0000100565, | 4 | 5875, 11040, 117, 23117 | 1.52 | 110 | 0.016 | 117 | 1 | 1 | 1 | 2 |
| Mecom | TRCN0000098096, | 10 | 8931, 9701, 357, 5860, 25749, | 1.63 | 13 | 0.016 | 118 | 2 | 2 | 4 | 8 |
| | TRCN0000085074, | | 31640, 1480, 9027, 1084, 120 | | | | | | | | |
| Sqle | TRCN0000076685, | 4 | 1441, 643, 8581, 14751 | 1.52 | 112 | 0.016 | 119 | 0 | 1 | 2 | 3 |
| Defa-rs7 | TRCN0000077067, | 3 | 18390, 320, 8794 | 1.48 | 194 | 0.016 | 120 | 1 | 1 | 1 | 2 |
| Chmp4c | TRCN0000105559, | 5 | 19371, 2564, 14352, 664, 901 | 1.55 | 76 | 0.016 | 121 | 0 | 2 | 3 | 3 |
| Hs3st1 | TRCN0000098198, | 5 | 7791, 11909, 8152, 4877, 109 | 1.55 | 77 | 0.016 | 122 | 1 | 1 | 2 | 4 |
| Plscr2 | TRCN0000105231, | 3 | 15301, 23460, 298 | 1.48 | 196 | 0.016 | 123 | 1 | 1 | 1 | 1 |
| Pfkfb2 | TRCN0000024883, | 5 | 715, 10720, 13609, 16809, 17410 | 1.55 | 79 | 0.016 | 124 | 0 | 1 | 1 | 1 |
| Mras | TRCN0000077571, | 4 | 11757, 12372, 20365, 427 | 1.52 | 118 | 0.017 | 125 | 1 | 1 | 1 | 1 |
| Otfr1256 | TRCN0000030436, | 4 | 7266, 2389, 18465, 46 | 1.52 | 119 | 0.017 | 126 | 1 | 1 | 2 | 3 |
| Kif11 | TRCN0000091775, | 5 | 11116, 19341, 13627, 25988, 217 | 1.54 | 82 | 0.017 | 127 | 1 | 1 | 1 | 1 |
| Pja2 | TRCN0000041007, | 3 | 28, 29526, 3026 | 1.47 | 209 | 0.018 | 128 | 1 | 1 | 2 | 2 |
| Rasa3 | TRCN0000034354, | 5 | 13379, 9289, 16578, 11825, 615 | 1.54 | 85 | 0.018 | 129 | 0 | 1 | 1 | 2 |
| Hint2 | TRCN0000060750, | 4 | 8674, 25558, 569, 479 | 1.51 | 129 | 0.018 | 130 | 1 | 2 | 2 | 3 |
| Hnrnpa1 | TRCN0000055265, | 5 | 486, 31915, 33099, 24020, 32 | 1.54 | 88 | 0.018 | 131 | 2 | 2 | 2 | 2 |
| Gstm1 | TRCN0000103242, | 3 | 9418, 20057, 315 | 1.47 | 214 | 0.018 | 132 | 1 | 1 | 1 | 2 |
| Pspc1 | TRCN0000102472, | 2 | 29068, 60 | 1.41 | 397 | 0.018 | 133 | 1 | 1 | 1 | 1 |
| Hspa1a | TRCN0000008515, | 4 | 9242, 13558, 13356, 648 | 1.51 | 130 | 0.018 | 134 | 0 | 1 | 1 | 2 |
| Pcdhb14 | TRCN0000094195, | 5 | 8649, 10612, 27307, 107, 25596 | 1.54 | 89 | 0.018 | 135 | 1 | 1 | 1 | 2 |
| Tia1 | TRCN0000077158, | 3 | 17082, 11428, 703 | 1.47 | 217 | 0.019 | 136 | 0 | 1 | 1 | 1 |
| Tsc22d3 | TRCN0000085746, | 4 | 16556, 6963, 23104, 192 | 1.51 | 131 | 0.019 | 137 | 1 | 1 | 1 | 2 |
| Cacna1d | TRCN0000088879, | 4 | 17597, 14258, 538, 2280 | 1.51 | 132 | 0.019 | 138 | 0 | 1 | 2 | 2 |
| Fgf16 | TRCN0000067083, | 3 | 10727, 16608, 700 | 1.47 | 222 | 0.019 | 139 | 0 | 1 | 1 | 1 |
| Zmynd8 | TRCN0000088515, | 4 | 8308, 20728, 25430, 139 | 1.51 | 135 | 0.019 | 140 | 1 | 1 | 1 | 2 |
| LOC436224 | TRCN0000090159, | 4 | 14954, 13404, 14572, 1722 | 1.51 | 136 | 0.019 | 141 | 0 | 0 | 1 | 1 |
| Dhdds | TRCN0000076081, | 3 | 12315, 20422, 455 | 1.47 | 224 | 0.019 | 142 | 1 | 1 | 1 | 1 |
| Mrps5 | TRCN0000104208, | 4 | 14273, 1535, 19771, 1311 | 1.51 | 138 | 0.019 | 143 | 0 | 0 | 2 | 2 |
| Mocs2 | TRCN0000076258, | 4 | 20843, 471, 7692, 966 | 1.51 | 139 | 0.019 | 144 | 1 | 2 | 2 | 3 |
| Rab35 | TRCN0000100533, | 5 | 11106, 2861, 8745, 430, 165 | 1.53 | 94 | 0.019 | 145 | 2 | 2 | 3 | 4 |
| Zeb1 | TRCN0000070822, | 5 | 13623, 12678, 29498, 3511, 74 | 1.53 | 96 | 0.019 | 146 | 1 | 1 | 2 | 2 |
| Fbxo16 | TRCN0000098960, | 4 | 7372, 12648, 9914, 309 | 1.51 | 141 | 0.02 | 147 | 1 | 1 | 1 | 3 |
| Gm5308 | TRCN0000091365, | 3 | 15252, 1465, 2217 | 1.46 | 230 | 0.02 | 148 | 0 | 0 | 2 | 2 |
| Kcnj2 | TRCN0000069704, | 2 | 23914, 163 | 1.4 | 406 | 0.02 | 149 | 1 | 1 | 1 | 1 |
| Krt33b | TRCN0000090466, | 4 | 14598, 20944, 28385, 145 | 1.51 | 143 | 0.02 | 150 | 1 | 1 | 1 | 1 |
| Asrgl1 | TRCN0000032310, | 5 | 10146, 21355, 16236, 13221, 519 | 1.53 | 101 | 0.021 | 151 | 0 | 1 | 1 | 1 |
| Phkg2 | TRCN0000024369, | 4 | 8383, 151, 20936, 25288 | 1.5 | 147 | 0.021 | 152 | 1 | 1 | 1 | 2 |
| 2610008E11Rik | TRCN0000095895, | 5 | 22476, 29796, 4782, 16465, 81 | 1.53 | 102 | 0.021 | 153 | 1 | 1 | 2 | 2 |
| Slc36a4 | TRCN0000068414, | 5 | 9735, 15454, 12410, 433, 2437 | 1.53 | 104 | 0.021 | 154 | 1 | 1 | 2 | 3 |
| Coro2b | TRCN0000090500, | 5 | 9954, 16387, 17836, 1493, 1583 | 1.52 | 108 | 0.021 | 155 | 0 | 0 | 2 | 3 |
| Slc35f2 | TRCN0000068900, | 3 | 16769, 17169, 1015 | 1.46 | 238 | 0.022 | 156 | 0 | 0 | 1 | 1 |
| Has1 | TRCN0000028846, | 3 | 12852, 9019, 544 | 1.46 | 240 | 0.022 | 157 | 0 | 1 | 1 | 2 |
| Zfp276 | TRCN0000081880, | 4 | 15222, 8387, 1482, 1332 | 1.5 | 154 | 0.022 | 158 | 0 | 0 | 2 | 3 |
| Casp9 | TRCN0000012249, | 5 | 6008, 24968, 132, 14897, 6537 | 1.52 | 111 | 0.022 | 159 | 1 | 1 | 1 | 3 |
| Hrsp12 | TRCN0000096921, | 5 | 14632, 22651, 26904, 13153, 220 | 1.52 | 114 | 0.022 | 160 | 1 | 1 | 1 | 1 |
| Zfp85-rs1 | TRCN0000096132, | 5 | 1114, 65, 1628, 25701, 31608 | 1.52 | 116 | 0.022 | 161 | 1 | 1 | 3 | 3 |
| Rab5c | TRCN0000100745, | 4 | 10722, 341, 22561, 18698 | 1.5 | 156 | 0.022 | 162 | 1 | 1 | 1 | 1 |
| Ren1 | TRCN0000030485, | 4 | 9911, 3960, 102, 7590 | 1.5 | 158 | 0.023 | 163 | 1 | 1 | 2 | 4 |
| Appbp2 | TRCN0000100448, | 4 | 10859, 2416, 253, 2743 | 1.5 | 159 | 0.023 | 164 | 1 | 1 | 3 | 3 |
| S100310 | TRCN0000097666, | 5 | 23088, 22029, 4738, 366, 428 | 1.52 | 117 | 0.023 | 165 | 2 | 2 | 3 | 3 |
| Neu1 | TRCN0000101688, | 5 | 20417, 7835, 582, 2000, 1885 | 1.52 | 120 | 0.023 | 166 | 0 | 1 | 3 | 4 |
| Adh7 | TRCN0000042021, | 2 | 10105, 515 | 1.4 | 428 | 0.023 | 167 | 0 | 1 | 1 | 1 |
| Rab1 | TRCN0000100863, | 3 | 12714, 14124, 1188 | 1.45 | 253 | 0.023 | 168 | 0 | 0 | 1 | 1 |
| Gpr88 | TRCN0000027956, | 3 | 22988, 28177, 104 | 1.45 | 255 | 0.023 | 169 | 1 | 1 | 1 | 1 |
| Atp8b3 | TRCN0000101401, | 5 | 17210, 9771, 18581, 464, 8738 | 1.52 | 123 | 0.023 | 170 | 1 | 1 | 1 | 3 |
| Strap | TRCN0000088835, | 5 | 19733, 13494, 25813, 351, 1340 | 1.52 | 124 | 0.023 | 171 | 1 | 1 | 2 | 2 |
| Wfikkn1 | TRCN0000092566, | 5 | 13674, 12444, 2610, 12676, 807 | 1.52 | 125 | 0.024 | 172 | 0 | 1 | 2 | 2 |
| Prss16 | TRCN0000032524, | 5 | 19108, 1105, 123, 19116, 30956 | 1.52 | 126 | 0.024 | 173 | 1 | 1 | 2 | 2 |
| Hist1h2bb | TRCN0000093064, | 5 | 14887, 2864, 16213, 2541, 3474 | 1.51 | 127 | 0.024 | 174 | 0 | 0 | 3 | 3 |
| Myl6 | TRCN0000090212, | 3 | 12615, 22219, 426 | 1.45 | 268 | 0.024 | 175 | 1 | 1 | 1 | 1 |
| Cul1 | TRCN0000012772, | 5 | 14909, 28731, 1150, 595, 1542 | 1.51 | 128 | 0.024 | 176 | 0 | 1 | 3 | 3 |
| Sesn3 | TRCN0000088250, | 4 | 15574, 22547, 979, 1450 | 1.49 | 188 | 0.024 | 177 | 0 | 1 | 2 | 2 |
| Tnfrsf9 | TRCN0000066541, | 4 | 15394, 6089, 1471, 403 | 1.49 | 189 | 0.025 | 178 | 1 | 1 | 2 | 3 |
| Slc12a2 | TRCN0000055390, | 4 | 18751, 19466, 24188, 291 | 1.49 | 172 | 0.025 | 179 | 1 | 1 | 1 | 1 |
| C1ra | TRCN0000031474, | 2 | 25913, 140 | 1.39 | 440 | 0.025 | 180 | 1 | 1 | 1 | 1 |
| Nfe2l2 | TRCN0000012131, | 9 | 11732, 13344, 1687, 24782, 28676, 1 [symbol] | 1.57 | 60 | 0.025 | 181 | 1 | 1 | 3 | 3 |
| | TRCN0000012130, | | | | | | | | | | |
| Slco4c1 | TRCN0000079265, | 3 | 8400, 30571, 89 | 1.45 | 277 | 0.025 | 182 | 1 | 1 | 1 | 2 |
| H60a | TRCN0000086894, | 5 | 21073, 19729, 27589, 61, 2481 | 1.51 | 133 | 0.025 | 183 | 1 | 1 | 2 | 2 |
| Tssk1 | TRCN0000024357, | 4 | 7880, 24712, 32333, 82 | 1.49 | 176 | 0.025 | 184 | 1 | 1 | 1 | 2 |
| Sox5 | TRCN0000075494, | 5 | 8045, 15922, 5925, 10679, 273 | 1.51 | 140 | 0.025 | 185 | 1 | 1 | 1 | 3 |
| Tspan33 | TRCN0000094713, | 2 | 31106, 56 | 1.39 | 444 | 0.026 | 186 | 1 | 1 | 1 | 1 |
| Col9a3 | TRCN0000091629, | 3 | 8207, 14579, 653 | 1.45 | 284 | 0.026 | 187 | 0 | 1 | 1 | 2 |
| Slc39a12 | TRCN0000079773, | 5 | 16807, 6177, 24125, 25267, 147 | 1.51 | 142 | 0.026 | 188 | 1 | 1 | 1 | 2 |
| Ddx3y | TRCN0000103639, | 5 | 14514, 29977, 6660, 173, 1008 | 1.5 | 144 | 0.026 | 189 | 1 | 1 | 2 | 3 |
| Vps54 | TRCN0000092069, | 3 | 153, 31471, 434 | 1.44 | 287 | 0.026 | 180 | 2 | 2 | 2 | 2 |
| Baz2a | TRCN0000075424, | 4 | 10448, 11795, 260, 26228 | 1.48 | 184 | 0.026 | 191 | 1 | 1 | 1 | 1 |
| LOC434449 | TRCN0000091814, | 5 | 20849, 10406, 9990, 7648, 324 | 1.5 | 145 | 0.026 | 192 | 1 | 1 | 1 | 3 |
| P [symbol] nc1 | TRCN0000078950, | 5 | 13855, 15062, 17954, 10638, 1122 | 1.5 | 146 | 0.027 | 193 | 0 | 0 | 1 | 1 |
| Icam2 | TRCN0000065976, | 3 | 25077, 24261, 142 | 1.44 | 289 | 0.027 | 194 | 1 | 1 | 1 | 1 |
| Kdelr3 | TRCN0000093592, | 4 | 22227, 19120, 2083, 207 | 1.48 | 185 | 0.027 | 195 | 1 | 1 | 2 | 2 |
| Gm1914 | TRCN0000087502, | 5 | 12178, 17047, 9941, 7728, 512 | 1.5 | 150 | 0.027 | 196 | 0 | 1 | 1 | 3 |
| Ptprq | TRCN0000080585, | 5 | 771, 15452, 420, 31836, 1195 | 1.5 | 151 | 0.027 | 187 | 1 | 2 | 3 | 3 |
| Gm12597 | TRCN0000066131, | 4 | 13019, 10359, 24333, 365 | 1.48 | 189 | 0.028 | 198 | 1 | 1 | 1 | 1 |
| Cryaa | TRCN0000097273, | 3 | 9277, 2297, 116 | 1.44 | 294 | 0.028 | 199 | 1 | 1 | 2 | 3 |
| Morc3 | TRCN0000025918, | 5 | 9124, 5196, 15133, 128, 28743 | 1.5 | 153 | 0.028 | 200 | 1 | 1 | 1 | 3 |

[symbol]

indicates data missing or illegible when filed

TABLE 3
Top 200 genes by W2ndB

| | | | | | | | | # Hairpins | # Hairpins | # Hairpins | # Hairpins |
|---|---|---|---|---|---|---|---|---|---|---|---|
| Gene | Hairpins | # Hairpins | Hairpin ranks | NES | Gene rank | p-value | p-value rank | 500 | 1000 | 5000 | 10000 |
| Hsh2d | TRCN0000097347, | 5 | 20171, 42, 17, 3469, 1308 | 0.002 | 1 | 0.00003 | 1 | 2 | 2 | 4 | 4 |
| Pdcl3 | TRCN0000092490, | 4 | 9302, 251, 30142, 18 | 0.0094 | 2 | 0.00022 | 2 | 2 | 2 | 2 | 3 |
| Zfat | TRGN0000086360, | 5 | 34131, 1445, 208, 34245, 45 | 0.0095 | 3 | 0.00033 | 3 | 2 | 2 | 3 | 3 |
| Ep300 | TRCN0000071205, | 5 | 9270, 28544, 98, 3200, 211 | 0.01 | 4 | 0.00039 | 5 | 2 | 2 | 3 | 4 |
| Hunk | TRCN0000024229, | 5 | 27348, 22962, 179, 33854, 203 | 0.011 | 5 | 0.00045 | 6 | 2 | 2 | 2 | 2 |
| LOC433318 | TRCN0000087049, | 4 | 242, 33184, 30426, 198 | 0.011 | 6 | 0.00034 | 4 | 2 | 2 | 2 | 2 |
| Med10 | TRCN0000082032, | 4 | 26707, 188, 27196, 271 | 0.012 | 7 | 0.00047 | 7 | 2 | 2 | 2 | 2 |
| Tnfrsf8 | TRCN0000066487, | 5 | 19, 9783, 280, 5877, 5922 | 0.012 | 8 | 0.0005 | 8 | 2 | 2 | 2 | 5 |
| Skp1a | TRCN0000012733, | 4 | 11352, 246, 33588, 292 | 0.014 | 9 | 0.00056 | 9 | 2 | 2 | 2 | 2 |
| Zfp119a | TRCN0000085401, | 5 | 3213, 144, 1232, 939, 287 | 0.014 | 10 | 0.00065 | 11 | 2 | 3 | 5 | 5 |
| Sh2d1b1 | TRCN0000081259, | 5 | 15974, 6119, 303, 125, 1572 | 0.015 | 11 | 0.00067 | 12 | 2 | 2 | 3 | 4 |
| Terf2 | TRCN0000071304, | 5 | 27110, 27310, 40, 15699, 338 | 0.015 | 12 | 0.00071 | 13 | 2 | 2 | 2 | 2 |
| Vps54 | TRCN0000092069, | 3 | 153, 31471, 434 | 0.015 | 13 | 0.00058 | 10 | 2 | 2 | 2 | 2 |
| Ppargc1a | TRCN0000095310, | 5 | 13882, 337, 29127, 76, 28575 | 0.015 | 14 | 0.00075 | 15 | 2 | 2 | 2 | 2 |
| Dgki | TRCN0000025437, | 5 | 354, 31017, 2028, 26, 34166 | 0.016 | 15 | 0.00075 | 16 | 2 | 2 | 3 | 3 |
| Fabp9 | TRCN0000101336, | 4 | 23270, 416, 32119, 33 | 0.016 | 16 | 0.00074 | 14 | 2 | 2 | 2 | 2 |
| Tfpi2 | TRCN0000080031, | 5 | 12576, 285, 6502, 558, 254 | 0.016 | 17 | 0.00079 | 17 | 2 | 3 | 3 | 4 |
| Arhgap8 | TRCN0000097305, | 5 | 15079, 264, 27349, 7684, 302 | 0.017 | 18 | 0.00095 | 19 | 2 | 2 | 2 | 3 |
| Krt78 | TRCN0000091189, | 5 | 5226, 19437, 135, 24499, 355 | 0.017 | 19 | 0.001 | 20 | 2 | 2 | 2 | 3 |
| Pten | TRCN0000028993, | 4 | 5080, 98, 7407, 481 | 0.019 | 20 | 0.001 | 21 | 2 | 2 | 2 | 4 |
| Ppp2r4 | TRCN0000077223, | 5 | 27244, 7023, 113, 16308, 436 | 0.02 | 21 | 0.0013 | 22 | 2 | 2 | 2 | 3 |
| Rab35 | TRCN0000100533, | 5 | 11106, 2861, 8745, 430, 165 | 0.021 | 22 | 0.0014 | 23 | 2 | 2 | 3 | 4 |
| Atr | TRGN0000023913, | 5 | 16392, 28370, 30775, 370, 350 | 0.021 | 23 | 0.0014 | 24 | 2 | 2 | 2 | 2 |
| Hnrnpa1 | TRCN0000055265, | 5 | 488, 31915, 33099, 24020, 32 | 0.021 | 24 | 0.0015 | 25 | 2 | 2 | 2 | 2 |
| Zfp828 | TRCN0000071361, | 5 | 23408, 2859, 171, 33618, 443 | 0.021 | 25 | 0.0015 | 26 | 2 | 2 | 3 | 3 |
| S100a10 | TRCN0000097666, | 5 | 23088, 22029, 4738, 366, 428 | 0.024 | 26 | 0.0018 | 29 | 2 | 2 | 3 | 3 |
| Ncor1 | TRCN0000096476, | 5 | 18426, 11856, 8890, 521, 99 | 0.024 | 27 | 0.0019 | 30 | 1 | 2 | 2 | 3 |
| Ankrd7 | TRCN0000103882, | 5 | 24725, 7505, 329, 19140, 452 | 0.024 | 28 | 0.0019 | 31 | 2 | 2 | 2 | 3 |
| Ly6a | TRCN0000100120, | 4 | 8348, 1478, 655, 162 | 0.026 | 29 | 0.0017 | 27 | 1 | 2 | 3 | 4 |
| Gtf2a1l | TRCN0000082113, | 5 | 7810, 546, 2136, 236, 33524 | 0.027 | 30 | 0.0024 | 33 | 1 | 2 | 3 | 4 |
| Hint2 | TRCN0000080750, | 4 | 8674, 25558, 569, 479 | 0.027 | 31 | 0.0018 | 28 | 1 | 2 | 2 | 3 |
| Mecom | TRCN0000096096, | 10 | 8931, 9701, 357, 5860, 25749, 31640, | 0.029 | 32 | 0.0043 | 47 | 2 | 2 | 4 | 8 |
| | TRCN0000085074, | | 1480, 9027, 1084, 120 | | | | | | | | |
| Gnptg | TRCN0000104629, | 5 | 2378, 24944, 31838, 701, 80 | 0.031 | 33 | 0.0033 | 37 | 1 | 2 | 3 | 3 |
| Icos | TRCN0000066859, | 5 | 17479, 5865, 1674, 658, 224 | 0.031 | 34 | 0.0033 | 38 | 1 | 2 | 3 | 4 |
| Gmeb1 | TRCN0000081631, | 3 | 15765, 496, 877 | 0.032 | 35 | 0.0022 | 32 | 1 | 2 | 2 | 2 |
| Cx3cr1 | TRCN0000026669, | 2 | 995, 756 | 0.033 | 36 | 0.00091 | 18 | 0 | 2 | 2 | 2 |
| Zkscan5 | TRCN0000086622, | 5 | 20736, 16621, 751, 88, 4807 | 0.033 | 37 | 0.0037 | 40 | 1 | 2 | 3 | 3 |
| Cd84 | TRCN0000066282, | 4 | 9400, 15105, 788, 425 | 0.034 | 38 | 0.0029 | 36 | 1 | 2 | 2 | 3 |
| Ptges3 | TRCN0000071289, | 5 | 8150, 2092, 1402, 738, 226 | 0.035 | 39 | 0.004 | 45 | 1 | 2 | 4 | 5 |
| Kif18a | TRCN0000091624, | 3 | 1078, 32425, 178 | 0.035 | 40 | 0.0027 | 34 | 1 | 1 | 2 | 2 |
| Cdc25a | TRCN0000009528, | 5 | 9853, 24552, 5719, 623, 835 | 0.036 | 41 | 0.0043 | 48 | 0 | 2 | 2 | 4 |
| Bcort1 | TRCN0000085730, | 5 | 24449, 326, 736, 2769, 33667 | 0.036 | 42 | 0.0043 | 49 | 1 | 2 | 3 | 3 |
| Atp1b1 | TRCN0000054955, | 3 | 14656, 1020, 435 | 0.035 | 43 | 0.0028 | 35 | 1 | 1 | 2 | 2 |
| Trim28 | TRCN0000071366, | 5 | 768, 270, 30216, 33362, 948 | 0.037 | 44 | 0.0045 | 51 | 1 | 3 | 3 | 3 |
| Pstk | TRCN0000098817, | 5 | 27746, 29556, 32125, 180, 818 | 0.038 | 45 | 0.0046 | 52 | 1 | 2 | 2 | 2 |
| Ift57 | TRCN0000100246, | 4 | 11465, 16596, 782, 749 | 0.038 | 46 | 0.0037 | 41 | 0 | 2 | 2 | 2 |
| Igsf8 | TRCN0000067489, | 4 | 23040, 810, 25041, 682 | 0.038 | 47 | 0.0037 | 42 | 0 | 2 | 2 | 2 |
| Fmo9 | TRCN0000099250, | 4 | 1948, 32182, 834, 680 | 0.039 | 48 | 0.0039 | 43 | 0 | 2 | 3 | 3 |
| Ptprq | TRCN0000080585, | 5 | 771, 15452, 420, 31836, 1195 | 0.039 | 49 | 0.0049 | 55 | 1 | 2 | 3 | 3 |
| Kir3dl1 | TRCN0000066961, | 5 | 33031, 4716, 29990, 688, 689 | 0.039 | 50 | 0.005 | 57 | 0 | 2 | 3 | 3 |
| Eny2 | TRCN0000086040, | 3 | 27030, 1115, 476 | 0.039 | 51 | 0.0034 | 39 | 1 | 1 | 2 | 2 |
| Podhb5 | TRCN0000095005, | 5 | 2646, 10797, 32674, 367, 808 | 0.04 | 52 | 0.0051 | 58 | 1 | 2 | 3 | 3 |
| Hif1a | TRCN0000054449, | 5 | 4919, 1617, 841, 29703, 305 | 0.04 | 53 | 0.0052 | 59 | 1 | 2 | 4 | 4 |
| Cd38 | TRCN0000068230, | 5 | 15467, 201, 2617, 894, 1456 | 0.041 | 54 | 0.0054 | 60 | 1 | 2 | 4 | 4 |
| Mocs2 | TRCN0000076258, | 4 | 20843, 471, 7892, 966 | 0.041 | 55 | 0.0044 | 50 | 1 | 2 | 2 | 3 |
| Gm5526 | TRCN0000089641, | 5 | 22283, 29255, 794, 537, 13104 | 0.042 | 56 | 0.0055 | 61 | 0 | 2 | 2 | 2 |
| LOC385190 | TRCN0000068341, | 5 | 5761, 778, 609, 3989, 5204 | 0.042 | 57 | 0.0056 | 63 | 0 | 2 | 3 | 5 |
| Cul5 | TRCN0000012796, | 5 | 937, 15007, 32911, 2014, 167 | 0.042 | 58 | 0.0057 | 65 | 1 | 2 | 3 | 3 |
| Prl | TRCN0000065892, | 5 | 917, 250, 7003, 28574, 4891 | 0.043 | 59 | 0.0058 | 66 | 1 | 2 | 3 | 4 |
| Slt3a | TRCN0000093714, | 5 | 3877, 691, 799, 4694, 2834 | 0.044 | 60 | 0.0062 | 68 | 0 | 2 | 5 | 5 |
| Ddx3y | TRCN0000103639, | 5 | 14514, 29977, 6660, 173, 1008 | 0.046 | 61 | 0.0067 | 70 | 1 | 1 | 2 | 3 |
| Ly6 [symbol] | TRCN0000100143, | 5 | 7989, 759, 29240, 29974, 816 | 0.046 | 62 | 0.0067 | 71 | 0 | 2 | 2 | 3 |
| Coro2a | TRCN0000090419, | 5 | 1034, 146, 33812, 33654, 2240 | 0.046 | 63 | 0.0069 | 73 | 1 | 1 | 3 | 3 |
| Pcmt1 | TRCN0000097399, | 5 | 15964, 7278, 926, 475, 29701 | 0.046 | 64 | 0.0069 | 74 | 1 | 2 | 2 | 3 |
| Zhx1 | TRCN0000070860, | 5 | 8264, 10211, 30700, 745, 854 | 0.047 | 65 | 0.0071 | 77 | 0 | 2 | 2 | 3 |
| LOC433453 | TRCN0000081297, | 4 | 17272, 972, 938, 5343 | 0.047 | 66 | 0.0056 | 64 | 0 | 2 | 2 | 3 |
| Zfp697 | TRCN0000086399, | 3 | 7117, 429, 1400 | 0.048 | 67 | 0.0049 | 54 | 1 | 1 | 2 | 3 |
| Rb1 | TRCN0000055378, | 6 | 9047, 7122, 2696, 2236, 21, 971 | 0.048 | 68 | 0.0084 | 93 | 1 | 2 | 4 | 6 |
| | TRCN0000055380, | | | | | | | | | | |
| Chmp4c | TRCN000010 [symbol] 9, | 5 | 19371, 2564, 14352, 664, 901 | 0.048 | 69 | 0.0073 | 80 | 0 | 2 | 3 | 3 |
| Zfp955a | TRCN0000104861, | 3 | 13945, 235, 1470 | 0.048 | 70 | 0.0049 | 56 | 1 | 1 | 2 | 2 |
| Zfp85-rs1 | TRCN0000096132, | 5 | 1114, 65, 1628, 25701, 31608 | 0.049 | 71 | 0.0074 | 83 | 1 | 1 | 3 | 3 |
| Ube2ql1 | TRCN0000092482, | 4 | 7069, 728, 1085, 3477 | 0.049 | 72 | 0.0061 | 67 | 0 | 1 | 3 | 4 |
| Prss16 | TRCN0000032524, | 5 | 19108, 1105, 123, 19116, 30956 | 0.049 | 73 | 0.0076 | 85 | 1 | 1 | 2 | 2 |
| Phlda2 | TRCN0000055085, | 5 | 11265, 17575, 987, 19321, 482 | 0.049 | 74 | 0.0076 | 86 | 1 | 2 | 2 | 2 |
| Nfe2 | TRCN0000081985, | 5 | 27993, 31616, 28479, 51, 1154 | 0.05 | 75 | 0.0079 | 87 | 1 | 1 | 2 | 2 |
| Gpr143 | TRCN0000027789, | 5 | 13081, 1081, 26679, 31704, 289 | 0.05 | 76 | 0.008 | 88 | 1 | 1 | 2 | 2 |
| Cdk13 | TRCN0000022984, | 5 | 5591, 4242, 22081, 31, 1173 | 0.051 | 77 | 0.008 | 89 | 1 | 1 | 3 | 4 |
| Zfp354c | TRCN0000082343, | 3 | 15588, 1606, 204 | 0.052 | 78 | 0.0056 | 62 | 1 | 1 | 2 | 2 |
| LOC436255 | TRCN0000092828, | 4 | 27117, 961, 1100, 31734 | 0.052 | 79 | 0.007 | 75 | 0 | 1 | 2 | 2 |
| Serpinb6d | TRCN0000086900, | 5 | 29170, 7353, 1183, 5985, 114 | 0.052 | 80 | 0.0085 | 94 | 1 | 1 | 2 | 4 |
| Fpgs | TRCN0000076255, | 4 | 9594, 1254, 33113, 592 | 0.053 | 81 | 0.0072 | 79 | 0 | 1 | 2 | 3 |
| Odf1 | TRCN0000098378, | 4 | 4047, 616, 24818, 1281 | 0.054 | 82 | 0.0073 | 82 | 0 | 1 | 3 | 3 |
| Vps18 | TRCN0000093227, | 5 | 17927, 980, 31434, 31118, 875 | 0.054 | 83 | 0.0092 | 101 | 0 | 2 | 2 | 2 |
| Clec2g | TRCN0000065795, | 4 | 24674, 72, 32680, 1459 | 0.054 | 84 | 0.0075 | 84 | 1 | 1 | 2 | 2 |
| Lifr | TRCN0000085815, | 5 | 32492, 1130, 490, 33565, 34164 | 0.065 | 85 | 0.0096 | 104 | 1 | 1 | 2 | 2 |
| Zfp192 | TRCN0000095899, | 5 | 25214, 24870, 32935, 934, 989 | 0.056 | 86 | 0.0097 | 105 | 0 | 2 | 2 | 2 |
| Enpp6 | TRCN0000081041, | 5 | 3768, 583, 32087, 31684, 1112 | 0.056 | 87 | 0.0098 | 107 | 0 | 1 | 3 | 3 |
| Gm270 | TRCN0000024033, | 5 | 18044, 5532, 964, 18411, 990 | 0.056 | 88 | 0.0098 | 108 | 0 | 2 | 2 | 3 |
| Slc1a6 | TRCN0000079897, | 3 | 1691, 29738, 390 | 0.056 | 89 | 0.0067 | 69 | 1 | 1 | 2 | 2 |
| Csmd1 | TRCN0000101371, | 3 | 31010, 856, 1548 | 0.057 | 90 | 0.0068 | 72 | 0 | 1 | 2 | 2 |
| Slfn2 | TRCN0000088366, | 4 | 719, 20729, 29279, 1319 | 0.057 | 91 | 0.0082 | 91 | 0 | 1 | 2 | 2 |
| Swap70 | TRCN0000100107, | 5 | 6516, 1083, 2766, 33685, 769 | 0.057 | 92 | 0.01 | 112 | 0 | 1 | 3 | 4 |
| Cul1 | TRCN0000012772, | 5 | 14909, 28731, 1150, 595, 1542 | 0.058 | 93 | 0.01 | 113 | 0 | 1 | 3 | 3 |
| Gsta2 | TRCN0000103296, | 5 | 14015, 1279, 29924, 34187, 215 | 0.058 | 94 | 0.01 | 114 | 1 | 1 | 2 | 2 |
| LOC381757 | TRCN0000025392, | 4 | 18804, 20444, 67, 1552 | 0.058 | 95 | 0.0084 | 92 | 1 | 1 | 2 | 2 |
| Prss2 | TRCN0000031997, | 5 | 14596, 30080, 959, 2875, 1051 | 0.059 | 96 | 0.011 | 120 | 0 | 1 | 3 | 3 |
| Tgm5 | TRCN0000104555, | 3 | 5831, 1176, 1504 | 0.059 | 97 | 0.0072 | 78 | 0 | 0 | 2 | 3 |
| Tnfrsl9 | TRCN0000086541, | 4 | 15364, 6089, 1471, 403 | 0.059 | 98 | 0.0037 | 95 | 1 | 1 | 2 | 3 |
| Pml | TRCN0000040483, | 3 | 5125, 1716, 621 | 0.06 | 99 | 0.0073 | 81 | 0 | 1 | 2 | 3 |
| Alg12 | TRCN0000098808, | 4 | 1296, 17759, 25677, 1010 | 0.06 | 100 | 0.009 | 96 | 0 | 0 | 2 | 2 |
| Prss38 | TRCN0000092125, | 5 | 26665, 6167, 31770, 606, 1203 | 0.06 | 101 | 0.011 | 121 | 0 | 1 | 2 | 3 |
| Cldn12 | TRCN0000092013, | 4 | 28600, 27942, 1515, 397 | 0.06 | 102 | 0.0091 | 98 | 1 | 1 | 2 | 2 |
| Rock2 | TRCN0000022920, | 4 | 5314, 407, 4815, 1513 | 0.06 | 103 | 0.0091 | 97 | 1 | 1 | 3 | 4 |
| Zfp3 | TRCN0000095883, | 4 | 876, 26097, 6482, 1360 | 0.061 | 104 | 0.0092 | 99 | 0 | 1 | 2 | 3 |
| Sqle | TRCN0000076685, | 4 | 1441, 643, 8581, 14751 | 0.061 | 105 | 0.0092 | 100 | 0 | 1 | 2 | 3 |
| Dek | TRCN0000086422, | 5 | 29061, 7135, 1283, 3590, 498 | 0.062 | 106 | 0.012 | 123 | 1 | 1 | 3 | 4 |
| Rnf38 | TRCN0000040999, | 4 | 24928, 968, 1372, 2269 | 0.062 | 107 | 0.0096 | 103 | 0 | 1 | 3 | 3 |
| Strap | TRCN0000088835, | 5 | 19733, 13494, 25813, 351, 1340 | 0.062 | 108 | 0.012 | 124 | 1 | 1 | 2 | 2 |
| LOC272661 | TRCN0000037122, | 2 | 2078, 973 | 0.063 | 109 | 0.0039 | 44 | 0 | 1 | 2 | 2 |
| Erap1 | TRCN0000031121, | 4 | 11025, 1589, 381, 34261 | 0.063 | 110 | 0.01 | 109 | 1 | 1 | 2 | 2 |
| C130060K24Rik | TRCN0000027805, | 4 | 1047, 13462, 1368, 6736 | 0.063 | 111 | 0.01 | 110 | 0 | 0 | 2 | 3 |
| Acadl | TRCN0000041272, | 2 | 2197, 753 | 0.064 | 112 | 0.004 | 46 | 0 | 1 | 2 | 2 |
| Aldh3a1 | TRCN0000042078, | 4 | 10215, 30707, 323, 1648 | 0.064 | 113 | 0.01 | 115 | 1 | 1 | 2 | 2 |
| Btk | TRCN0000023689, | 4 | 5582, 6842, 1011, 1421 | 0.064 | 114 | 0.01 | 116 | 0 | 0 | 2 | 4 |
| Pex12 | TRCN0000040529, | 5 | 11257, 22831, 874, 1223, 32217 | 0.065 | 115 | 0.013 | 131 | 0 | 1 | 2 | 2 |
| Ube4b | TRCN0000008437, | 4 | 5241, 13682, 1250, 1359 | 0.065 | 116 | 0.011 | 118 | 0 | 0 | 2 | 3 |
| Sesn3 | TRCN0000088250, | 4 | 15574, 22547, 979, 1450 | 0.065 | 117 | 0.011 | 117 | 0 | 1 | 2 | 2 |
| Syt12 | TRCN0000093163, | 5 | 13924, 21197, 1181, 1046, 6980 | 0.065 | 118 | 0.013 | 135 | 0 | 0 | 2 | 3 |
| Sncaip | TRCN0000085373, | 5 | 5938, 21328, 28062, 1027, 1189 | 0.065 | 119 | 0.013 | 136 | 0 | 0 | 2 | 3 |
| Rif1 | TRCN0000071338, | 5 | 23109, 885, 1237, 3758, 32928 | 0.065 | 120 | 0.013 | 139 | 0 | 1 | 3 | 3 |
| Pgk2 | TRCN0000025433, | 5 | 32491, 30416, 16549, 286, 1452 | 0.066 | 121 | 0.014 | 142 | 1 | 1 | 2 | 2 |
| Klra2 | TRCN0000094803, | 5 | 3315, 6816, 956, 23186, 1248 | 0.067 | 122 | 0.014 | 143 | 0 | 1 | 3 | 4 |
| Nox3 | TRCN0000076593, | 3 | 1729, 1423, 22393 | 0.068 | 123 | 0.0095 | 102 | 0 | 0 | 2 | 2 |
| Arsa | TRCN0000101487, | 3 | 22458, 2101, 309 | 0.069 | 124 | 0.0097 | 106 | 1 | 1 | 2 | 2 |
| Gtf2e1 | TRCN0000085170, | 5 | 17453, 25986, 2612, 1331, 846 | 0.069 | 125 | 0.015 | 147 | 0 | 1 | 3 | 3 |
| Il1a | TRCN0000067052, | 5 | 1543, 9798, 7260, 26811, 269 | 0.07 | 126 | 0.015 | 149 | 1 | 1 | 2 | 4 |
| Akr1c14 | TRCN0000099615, | 4 | 20842, 20782, 1641, 786 | 0.07 | 127 | 0.012 | 125 | 0 | 1 | 2 | 2 |
| 2310008H04Rik | TRCN0000104163, | 5 | 3883, 958, 33910, 1320, 19968 | 0.07 | 128 | 0.015 | 151 | 0 | 1 | 3 | 3 |
| Psmd6 | TRCN0000066211, | 4 | 873, 9715, 1636, 20822 | 0.07 | 129 | 0.012 | 126 | 0 | 1 | 2 | 3 |
| Hp1bp3 | TRCN0000093004, | 5 | 4610, 12751, 674, 13385, 1424 | 0.07 | 130 | 0.016 | 157 | 0 | 1 | 3 | 3 |
| Etv4 | TRCN0000055132, | 3 | 20280, 2169, 322 | 0.071 | 131 | 0.01 | 111 | 1 | 1 | 2 | 2 |
| Dok2 | TRCN0000077432, | 5 | 14484, 21493, 1294, 16506, 1073 | 0.071 | 132 | 0.016 | 158 | 0 | 0 | 2 | 2 |
| Zfp276 | TRCN0000081880, | 4 | 15222, 8387, 1482, 1332 | 0.071 | 133 | 0.013 | 127 | 0 | 0 | 2 | 3 |
| Trim59 | TRCN0000040932, | 2 | 1059, 2366 | 0.071 | 134 | 0.0048 | 53 | 0 | 0 | 2 | 2 |
| Erbb2 | TRCN0000023386, | 4 | 27875, 21966, 520, 1765 | 0.071 | 135 | 0.013 | 128 | 0 | 1 | 2 | 2 |
| Pcdhb15 | TRCN0000094447, | 5 | 32682, 9262, 16, 8485, 1670 | 0.072 | 136 | 0.016 | 161 | 1 | 1 | 2 | 4 |
| Zfp292 | TRCN0000099729, | 5 | 38, 30015, 4872, 1667, 2634 | 0.072 | 137 | 0.016 | 162 | 1 | 1 | 4 | 4 |
| Spcs1 | TRCN0000018439, | 5 | 8531, 1561, 33459, 377, 34205 | 0.072 | 138 | 0.016 | 165 | 1 | 1 | 2 | 3 |
| Ssh2 | TRCN0000081501, | 4 | 31136, 1893, 222, 18409 | 0.072 | 139 | 0.013 | 132 | 1 | 1 | 2 | 2 |
| Mrps5 | TRCN0000104208, | 4 | 14273, 1535, 19771, 1311 | 0.072 | 140 | 0.013 | 134 | 0 | 0 | 2 | 2 |
| Sult1d1 | TRCN0000103272, | 5 | 17571, 17805, 16728, 152, 1642 | 0.072 | 141 | 0.016 | 166 | 1 | 1 | 2 | 2 |
| Cryaa | TRCN0000097273, | 3 | 9277, 2297, 118 | 0.072 | 142 | 0.011 | 119 | 1 | 1 | 2 | 3 |
| Scamp3 | TRCN0000105364, | 4 | 14146, 225, 15537, 1911 | 0.073 | 143 | 0.013 | 137 | 1 | 1 | 2 | 2 |
| Clk1 | TRCN0000023109, | 5 | 1498, 3991, 28169, 687, 33457 | 0.074 | 144 | 0.017 | 171 | 0 | 1 | 3 | 3 |
| Trav7-4 | TRCN0000099736, | 5 | 5312, 1557, 33895, 33549, 511 | 0.074 | 145 | 0.017 | 170 | 0 | 1 | 2 | 3 |
| Wnt2 | TRCN0000042624, | 7 | 1065, 10872, 16741, 26138, 20457, | 0.074 | 146 | 0.021 | 206 | 0 | 1 | 2 | 2 |
| | TRCN0000042625, | | 28091, 832 | | | | | | | | |
| Fkbp1a | TRCN0000012489, | 5 | 11893, 848, 3406, 21158, 1466 | 0.075 | 147 | 0.017 | 173 | 0 | 1 | 3 | 3 |
| Caml | TRCN0000100981, | 4 | 13117, 1690, 4922, 1152 | 0.076 | 148 | 0.015 | 144 | 0 | 0 | 3 | 3 |
| Zxdc | TRCN0000104858, | 4 | 24542, 16286, 261, 1992 | 0.076 | 149 | 0.015 | 145 | 1 | 1 | 2 | 2 |
| Cd160 | TRCN0000066917, | 4 | 15779, 16462, 941, 1767 | 0.076 | 150 | 0.015 | 146 | 0 | 1 | 2 | 2 |
| Lgals9 | TRCN0000066438, | 5 | 22590, 9427, 1390, 1217, 3047 | 0.077 | 151 | 0.018 | 182 | 0 | 0 | 3 | 4 |
| Il1f9 | TRCN0000067354, | 5 | 7091, 1508, 23332, 893, 30963 | 0.077 | 152 | 0.019 | 183 | 0 | 1 | 2 | 3 |
| Rnf146 | TRCN0000040723, | 5 | 1793, 3390, 3763, 6435, 53 | 0.077 | 153 | 0.019 | 184 | 1 | 1 | 4 | 5 |
| Chrnb2 | TRCN0000102860, | 4 | 13041, 1570, 27619, 1599 | 0.078 | 154 | 0.015 | 150 | 0 | 0 | 2 | 2 |
| Matk | TRCN0000023425, | 4 | 11387, 8385, 1956, 518 | 0.078 | 155 | 0.015 | 152 | 0 | 1 | 2 | 3 |
| Zfp715 | TRCN0000086214, | 4 | 24212, 5813, 1527, 1623 | 0.078 | 156 | 0.016 | 153 | 0 | 0 | 2 | 3 |
| Cetn2 | TRCN0000090949, | 4 | 31668, 1774, 1627, 1521 | 0.078 | 157 | 0.016 | 154 | 0 | 0 | 3 | 3 |
| Il20ra | TRCN0000067927, | 4 | 1099, 25653, 30797, 1771 | 0.078 | 158 | 0.016 | 155 | 0 | 0 | 2 | 2 |
| Klrc1 | TRCN0000066056, | 5 | 6946, 17194, 22566, 1108, 1469 | 0.079 | 159 | 0.019 | 190 | 0 | 0 | 2 | 3 |
| Actn4 | TRCN0000090215 | 5 | 3823, 28546, 1861, 228, 1764 | 0.079 | 160 | 0.019 | 191 | 1 | 1 | 4 | 4 |
| Gm4787 | TRCN0000092088, | 5 | 19169, 4864, 11031, 1569, 822 | 0.079 | 161 | 0.019 | 192 | 0 | 1 | 3 | 3 |
| Kdelr3 | TRCN0000093592, | 4 | 22227, 19120, 2083, 207 | 0.079 | 162 | 0.016 | 159 | 1 | 1 | 2 | 2 |
| Ntrk2 | TRCN0000023699, | 5 | 10518, 15897, 1585, 809, 29868 | 0.079 | 163 | 0.02 | 193 | 0 | 1 | 2 | 2 |
| Map2k7 | TRCN0000012508, | 4 | 1812, 18462, 32574, 1070 | 0.08 | 164 | 0.016 | 163 | 0 | 0 | 2 | 2 |
| Gtf2h2 | TRCN0000085831, | 5 | 10265, 4158, 3074, 1547, 944 | 0.08 | 165 | 0.02 | 196 | 0 | 1 | 4 | 4 |
| Cd79a | TRCN0000066142, | 4 | 12232, 26903, 1877, 881 | 0.08 | 166 | 0.016 | 164 | 0 | 1 | 2 | 2 |
| Rnf113a1 | TRCN0000040845, | 5 | 16509, 16168, 896, 29271, 1574 | 0.08 | 167 | 0.02 | 198 | 0 | 1 | 2 | 2 |
| Mybph | TRCN0000090224, | 4 | 16030, 1827, 22809, 1071 | 0.08 | 168 | 0.017 | 187 | 0 | 0 | 2 | 2 |
| Il19 | TRCN0000066954, | 5 | 21629, 29233, 15428, 1760, 348 | 0.08 | 169 | 0.02 | 199 | 1 | 1 | 2 | 2 |
| Dnajc7 | TRCN0000009545, | 5 | 483, 31879, 1717, 30519, 33840 | 0.08 | 170 | 0.02 | 200 | 1 | 1 | 2 | 2 |
| Rnf208 | TRCN0000040798, | 4 | 9868, 10905, 1978, 650 | 0.081 | 171 | 0.017 | 168 | 0 | 1 | 2 | 3 |
| Zbp1 | TRCN0000077362, | 4 | 300, 30076, 2122, 23207 | 0.082 | 172 | 0.017 | 172 | 1 | 1 | 2 | 2 |
| Gp6 | TRCN0000089059, | 5 | 1243, 1494, 28586, 24992, 27681 | 0.082 | 173 | 0.021 | 204 | 0 | 0 | 2 | 2 |
| Adck2 | TRCN0000023855, | 3 | 25963, 2062, 1712 | 0.082 | 174 | 0.013 | 129 | 0 | 0 | 2 | 2 |
| Ddx3x | TRCN0000103750, | 5 | 13588, 21642, 1679, 23129, 710 | 0.082 | 175 | 0.021 | 205 | 0 | 1 | 2 | 2 |
| Ssh3 | TRCN0000081232, | 4 | 17412, 27708, 1677, 1671 | 0.082 | 176 | 0.018 | 174 | 0 | 0 | 2 | 2 |
| Efnb1 | TRCN0000066445, | 4 | 8855, 1798, 29737, 1328 | 0.082 | 177 | 0.018 | 175 | 0 | 0 | 2 | 3 |
| Ybx2 | TRCN0000095323, | 4 | 16355, 7600, 1069, 1904 | 0.083 | 178 | 0.018 | 176 | 0 | 0 | 2 | 3 |
| Pbld1 | TRCN0000099508, | 3 | 23004, 1186, 2286 | 0.083 | 179 | 0.013 | 133 | 0 | 0 | 2 | 2 |
| Csfsrb2 | TRCN0000067077, | 4 | 9317, 1929, 1021, 3692 | 0.083 | 180 | 0.018 | 177 | 0 | 0 | 3 | 4 |
| Floxo3 | TRCN0000092061, | 4 | 2155, 8816, 30304, 372 | 0.084 | 181 | 0.018 | 178 | 1 | 1 | 2 | 3 |
| Poron | TRCN0000093489, | 4 | 17000, 1984, 903, 27728 | 0.084 | 182 | 0.016 | 179 | 0 | 1 | 2 | 2 |
| Gm5308 | TRCN0000091365, | 3 | 15252, 1465, 2217 | 0.084 | 183 | 0.013 | 140 | 0 | 0 | 2 | 2 |
| Tgtp1 | TRCN0000077399, | 5 | 17610, 30367, 1912, 17421, 154 | 0.084 | 184 | 0.022 | 216 | 1 | 1 | 2 | 2 |
| LOC436589 | TRCN0000087621, | 5 | 29315, 28049, 1720, 730, 4133 | 0.084 | 185 | 0.022 | 217 | 0 | 1 | 3 | 3 |
| LOC436127 | TRCN0000078999, | 4 | 1584, 24376, 31374, 1761 | 0.084 | 186 | 0.018 | 180 | 0 | 0 | 2 | 2 |
| Poteg | TRCN0000103811, | 3 | 28765, 1982, 2063 | 0.084 | 187 | 0.014 | 141 | 0 | 0 | 2 | 2 |
| Adar | TRCN0000071315, | 4 | 25676, 44, 31960, 2301 | 0.085 | 188 | 0.019 | 185 | 1 | 1 | 2 | 2 |
| Ceacam11 | TRCN0000094721, | 4 | 8742, 772, 206 [symbol] , 33117 | 0.085 | 189 | 0.019 | 186 | 0 | 1 | 2 | 3 |
| Rab33a | TRCN0000100728, | 4 | 14654, 2025, 32113, 892 | 0.085 | 190 | 0.019 | 187 | 0 | 1 | 2 | 2 |
| Tap1 | TRCN0000066348, | 5 | 7853, 594, 1796, 28924, 33003 | 0.085 | 191 | 0.023 | 219 | 0 | 1 | 2 | 3 |
| Dupd1 | TRCN0000081356, | 5 | 26434, 29425, 4491, 1595, 1233 | 0.086 | 192 | 0.023 | 222 | 0 | 0 | 3 | 3 |
| Mup3 | TRCN0000105507, | 5 | 19179, 16274, 1245, 4298, 1608 | 0.086 | 193 | 0.024 | 227 | 0 | 0 | 3 | 3 |
| Zfp764 | TRCN0000095294, | 5 | 1935, 23521, 28763, 28534, 295 | 0.087 | 194 | 0.024 | 229 | 1 | 1 | 2 | 2 |
| Ppptr15b | TRCN0000103621, | 4 | 4240, 31618, 1856, 1541 | 0.087 | 195 | 0.02 | 195 | 0 | 0 | 3 | 3 |
| Tbx4 | TRCN0000084571, | 5 | 1862, 14049, 5493, 560, 31317 | 0.088 | 196 | 0.024 | 232 | 0 | 1 | 2 | 3 |
| Tceb3 | TRCN0000081831, | 2 | 808, 3101 | 0.088 | 197 | 0.0071 | 76 | 0 | 1 | 2 | 2 |
| Olfr1255 | TRCN0000030436, | 4 | 7266, 2389, 18465, 46 | 0.088 | 198 | 0.02 | 201 | 1 | 1 | 2 | 3 |
| Pdxk | TRCN0000024849, | 3 | 2638, 21659, 685 | 0.089 | 199 | 0.015 | 148 | 0 | 1 | 2 | 2 |
| Neu1 | TRCN0000101688, | 5 | 20417, 7835, 582, 2000, 1885 | 0.089 | 200 | 0.025 | 235 | 0 | 1 | 3 | 4 |

[symbol]

indicates data missing or illegible when filed

TABLE 4
70 genes selected for validation

| | | | | | | | | # | # | # | # |
|---|---|---|---|---|---|---|---|---|---|---|---|
| | | # | | | Gene | | p-value | HP | HP | HP | HP |
| Gene | Hairpins (HP) | HP | HP ranks | NES | rank | p-value | rank | 500 | 1000 | 5000 | 10000 |
| Tsc2 | TRCN0000042724, | 4 | 1, 15189, 32410, 30320 | 1.620 | 13 | 0.0000 | 1 | 1 | 1 | 1 | 1 |
| Grid2ip | TRCN0000091223, | 5 | 19498, 35, 21740, 17629, 22028 | 1.650 | 1 | 0.0000 | 2 | 1 | 1 | 1 | 1 |
| Kcnk13 | TRCN0000065524, | 4 | 4343, 2, 23401, 32404 | 1.610 | 22 | 0.0007 | 3 | 1 | 1 | 2 | 2 |
| Pcgf5 | TRCN0000095159, | 5 | 25488, 24650, 6992, 6, 28132 | 1.640 | 2 | 0.0007 | 4 | 1 | 1 | 1 | 2 |
| Rasgrf1 | TRCN0000077588, | 5 | 29, 14263, 15074, 23758, 16254 | 1.640 | 3 | 0.0008 | 5 | 1 | 1 | 1 | 1 |
| Bmi1 | TRCN0000012563, | 5 | 22, 10903, 12794, 13009, 26452 | 1.640 | 4 | 0.0010 | 6 | 1 | 1 | 1 | 1 |
| Acx3 | TRCN0000076573, | 4 | 12424, 13463, 23039, 41 | 1.610 | 23 | 0.0010 | 7 | 1 | 1 | 1 | 1 |
| Cdkn2b | TRCN0000042598, | 5 | 3, 4898, 3592, 20735, 13040 | 1.640 | 5 | 0.0011 | 8 | 1 | 1 | 3 | 3 |
| Ptprg | TRCN0000029949, | 5 | 20133, 17145, 19169, 18687, 247 | 1.630 | 6 | 0.0012 | 9 | 1 | 1 | 1 | 1 |
| Cd83 | TRCN0000066673, | 5 | 9393, 31651, 25766, 10, 9722 | 1.630 | 7 | 0.0014 | 10 | 1 | 1 | 1 | 3 |
| Tuba3b | TRCN0000089883, | 5 | 39, 14756, 13568, 27986, 22107 | 1.630 | 9 | 0.0015 | 12 | 1 | 1 | 1 | 1 |
| Slc7a10 | TRCN0000079468, | 5 | 16026, 106, 19656, 17286, 11610 | 1.620 | 10 | 0.0016 | 14 | 1 | 1 | 1 | 1 |
| P1gdr | TRCN0000026600, | 5 | 13415, 54, 11337, 20978, 9174 | 1.620 | 12 | 0.0017 | 15 | 1 | 1 | 1 | 2 |
| Trmt2b | TRCN0000104020, | 5 | 24600, 16420, 36346, 5, 38219 | 1.620 | 14 | 0.0022 | 17 | 1 | 1 | 1 | 1 |
| Foxj1 | TRCN0000103560, | 5 | 20, 30619, 14909, 9954, 31389 | 1.610 | 17 | 0.0026 | 18 | 1 | 1 | 1 | 2 |
| Ntrk3 | TRCN0000023514, | 5 | 10991, 19577, 49, 9035, 8673 | 1.620 | 15 | 0.0026 | 19 | 1 | 1 | 1 | 3 |
| Tnfrsf8 | TRCN0000066483, | 5 | 5944, 280, 10537, 5899, 19 | 1.610 | 18 | 0.0026 | 20 | 2 | 2 | 2 | 4 |
| Calm4 | TRCN0000104615, | 5 | 181, 18860, 12031, 18175, 18117 | 1.610 | 16 | 0.0026 | 21 | 1 | 1 | 1 | 1 |
| Cmpk1 | TRCN0000025399, | 5 | 9819, 27184, 7, 7912, 39482 | 1.610 | 19 | 0.0028 | 23 | 1 | 1 | 1 | 3 |
| Pcdhb12 | TRCN0000094494, | 5 | 29370, 16828, 52, 25862, 15928 | 1.610 | 20 | 0.0030 | 24 | 1 | 1 | 1 | 1 |
| Pdhb | TRCN0000041828, | 5 | 17863, 47, 5207, 19428, 15278 | 1.610 | 21 | 0.0031 | 26 | 1 | 1 | 1 | 2 |
| Oplah | TRCN0000098845, | 5 | 30679, 15, 25677, 31665, 32462 | 1.600 | 24 | 0.0035 | 27 | 1 | 1 | 1 | 1 |
| Hcls1 | TRCN0000103625, | 5 | 15032, 9818, 21198, 27, 35880 | 1.600 | 25 | 0.0039 | 28 | 1 | 1 | 1 | 2 |
| Tfpi2 | TRCN0000080028, | 5 | 558, 285, 6532, 15472, 254 | 1.600 | 26 | 0.0039 | 29 | 2 | 3 | 3 | 4 |
| Hist1h1a | TRCN0000097050, | 4 | 13758, 18220, 196, 22530 | 1.580 | 41 | 0.0040 | 30 | 1 | 1 | 1 | 1 |
| Pdcl3 | TRCN0000092488, | 4 | 18, 35891, 9824, 251 | 1.580 | 42 | 0.0041 | 31 | 2 | 2 | 2 | 3 |
| Nedd8 | TRCN0000012738, | 4 | 17716, 57, 6815, 10368 | 1.580 | 44 | 0.0043 | 32 | 1 | 1 | 1 | 2 |
| Cd84 | TRCN0000066278, | 5 | 20568, 425, 20068, 789, 9964 | 1.600 | 28 | 0.0046 | 33 | 1 | 2 | 2 | 3 |
| Usp24 | TRCN0000040628, | 5 | 25315, 64, 23283, 27943, 8756 | 1.590 | 30 | 0.0050 | 36 | 1 | 1 | 1 | 2 |
| Gmeb1 | TRCN0000081629, | 4 | 496, 878, 21312, 14545 | 1.570 | 54 | 0.0053 | 38 | 1 | 2 | 2 | 2 |
| Pcdha4 | TRCN0000094349, | 4 | 25604, 15955, 90, 27325 | 1.570 | 53 | 0.0053 | 39 | 1 | 1 | 1 | 1 |
| Ncor1 | TRCN0000096474, | 5 | 99, 521, 24168, 6929, 14106 | 1.590 | 31 | 0.0054 | 40 | 1 | 2 | 2 | 3 |
| Nfib | TRCN0000012088, | 5 | 39676, 4, 32881, 5360, 38079 | 1.590 | 34 | 0.0056 | 41 | 1 | 1 | 1 | 2 |
| Ift57 | TRCN0000100245, | 5 | 750, 13390, 783, 22272, 21906 | 1.590 | 37 | 0.0056 | 44 | 0 | 2 | 2 | 2 |
| Terf2 | TRCN0000071303, | 5 | 33058, 32860, 21232 338, 40 | 1.590 | 36 | 0.0056 | 45 | 2 | 2 | 2 | 2 |
| Hsh2d | TRCN0000097344, | 5 | 1309, 17, 3472, 25916, 42 | 1.580 | 43 | 0.0066 | 47 | 2 | 2 | 4 | 4 |
| Flnb | TRCN0000091373, | 5 | 189, 19852, 21636, 28312, 23974 | 1.570 | 48 | 0.0069 | 51 | 1 | 1 | 1 | 1 |
| Fabp9 | TRCN0000101335, | 5 | 416, 29018, 33, 21594, 37868 | 1.570 | 55 | 0.0074 | 53 | 2 | 2 | 2 | 2 |
| Rasgrf2 | TRCN0000077593, | 5 | 11393, 342, 24178, 20170, 16569 | 1.570 | 56 | 0.0077 | 55 | 1 | 1 | 1 | 1 |
| Ikbke | TRCN0000026687, | 4 | 23391, 112, 9361, 8212 | 1.560 | 67 | 0.0077 | 56 | 1 | 1 | 1 | 3 |
| Acln2 | TRCN0000072023, | 4 | 111, 13387, 20077, 6121 | 1.550 | 69 | 0.0080 | 62 | 1 | 1 | 1 | 2 |
| Rab11a | TRCN0000100340, | 4 | 8569, 25090, 17719, 161 | 1.550 | 75 | 0.0084 | 64 | 1 | 1 | 1 | 2 |
| Ppp2r4 | TRCN0000077223, | 5 | 32995, 7071, 21968, 436, 113 | 1.560 | 62 | 0.0086 | 6 [symbol] | 2 | 2 | 2 | 3 |
| Ube1 | TRCN0000012743, | [symbol] | 20972, 23604, 23395, 29222, 200 | 1.550 | 70 | 0.0093 | 70 | 1 | 1 | 1 | 1 |
| Zkscan5 | TRCN0000086618, | 5 | 4819, 88, 22305, 752, 26484 | 1.550 | 71 | 0.0096 | 71 | 1 | 2 | 3 | 3 |
| Phlda2 | TRCN0000055083, | 5 | 988, 25075, 13005, 23292, 482 | 1.550 | 73 | 0.0098 | 73 | 1 | 2 | 2 | 2 |
| Pten | TRCN0000028989, | 5 | 9676, 481, 98, 7474, 5094 | 1.550 | 74 | 0.0098 | 74 | 2 | 2 | 2 | 5 |
| Arhgap8 | TRCN0000097304, | 5 | 7777, 20025, 33101, 302, 264 | 1.550 | 76 | 0.0098 | 75 | 2 | 2 | 2 | 3 |
| Ptges3 | TRCN0000071288, | 5 | 1403, 8296, 226, 739, 2094 | 1.550 | 79 | 0.0100 | 77 | 1 | 2 | 4 | 5 |
| Scamp3 | TRCN0000105360, | 5 | 225, 20801, 1913, 10854, 18406 | 1.550 | 81 | 0.0100 | 79 | 1 | 1 | 2 | 2 |
| Mark1 | TRCN0000024169, | 5 | 15197, 627, 15914, 21894, 16907 | 1.550 | 82 | 0.0110 | 82 | 0 | 1 | 1 | 1 |
| Cdc16 | TRCN0000088148, | 5 | 37743, 25, 7519, 37202, 22430 | 1.550 | 84 | 0.0110 | 84 | 1 | 1 | 1 | 2 |
| Ppargc1a | TRCN0000095309, | 5 | 34874, 17934, 76, 337, 34324 | 1.550 | 85 | 0.0110 | 85 | 2 | 2 | 2 | 2 |
| Rb1 | TRCN0000042546, | 6 | 21, 2238, 9455, 972, 7172, 2698 | 1.590 | 35 | 0.0120 | 91 | 1 | 2 | 4 | 6 |
| Tspan33 | TRCN0000094709, | 4 | 13254, 18162, 56, 36855 | 1.540 | 99 | 0.0120 | 92 | 1 | 1 | 1 | 1 |
| Dtymk | TRCN0000024614, | 5 | 27050, 28546, 100, 19621, 5760 | 1.540 | 91 | 0.0120 | 94 | 1 | 1 | 1 | 2 |
| Ugcg | TRCN0000093764, | 5 | 77, 34973, 27743, 22523, 24989 | 1.540 | 90 | 0.0120 | 95 | 1 | 1 | 1 | 1 |
| Cdk13 | TRCN0000022984, | 5 | 5607, 27828, 1174, 31, 4251 | 1.530 | 101 | 0.0130 | 104 | 1 | 1 | 3 | 4 |
| Bnip3l | TRCN0000009729, | 5 | 517, 14101, 14527, 20414, 11514 | 1.530 | 106 | 0.0140 | 108 | 0 | 1 | 1 | 1 |
| Med10 | TRCN0000082028, | 9 | 272, 32948, 188, 13302, 32454 | 1.530 | 104 | 0.0140 | 110 | 2 | 2 | 2 | 2 |
| Usp35 | TRCN0000092448, | 5 | 5648, 16380, 9846, 17400, 156 | 1.530 | 107 | 0.0140 | 111 | 1 | 1 | 1 | 3 |
| Ep300 | TRCN0000071203, | 5 | 96, 211, 9784, 3204, 32293 | 1.530 | 114 | 0.0150 | 116 | 2 | 2 | 3 | 4 |
| Dhdds | TRCN0000076079, | 4 | 455, 20339, 14983, 26169 | 1.520 | 120 | 0.0150 | 119 | 1 | 1 | 1 | 1 |
| Chek1 | TRCN0000012648, | 5 | 23815, 14869, 23858, 327, 26693 | 1.520 | 119 | 0.0150 | 120 | 1 | 1 | 1 | 1 |
| Mecom | TRCN0000085073, | 10 | 9282, 1482, 1085 | 1.600 | 29 | 0.0160 | 126 | 2 | 2 | 4 | 7 |
| Dok2 | TRCN0000077428, | 5 | 22181, 27242, 1074, 1295, 19031 | 1.520 | 124 | 0.0160 | 128 | 0 | 0 | 2 | 2 |
| Kdm4b | TRCN0000103535, | 5 | 91, 13340, 19256, 36360, 7653 | 1.510 | 131 | 0.0170 | 132 | 1 | 1 | 1 | 2 |
| Rab35 | TRCN0000100530, | 5 | 9022, 165, 2884, 12722, 430 | 1.610 | 135 | 0.0180 | 136 | 2 | 2 | 3 | 4 |
| Ptktb2 | TRCN0000024879, | 5 | 23131, 12068, 22508, 17420, 716 | 1.510 | 140 | 0.0190 | 140 | 0 | 1 | 1 | 1 |
| Cbr4 | TRCN0000099160, | 4 | 27820, 71, 26901, 4019 | 1.510 | 142 | 0.0190 | 145 | 1 | 1 | 2 | 2 |

[symbol]

indicates data missing or illegible when filed

TABLE 5
Validation hits

| | | | Null | |
|---|---|---|---|---|
| TRCN # | Symbol | WT ave-GR | ave-GR | ratio = wt/null |
| TRCN0000024614 | Dtymk | 0.53 | 0.13 | 4.16 |
| TRCN0000024616 | Dtymk | 0.83 | 0.28 | 2.96 |
| TRCN0000024615 | Dtymk | 0.85 | 0.51 | 1.67 |
| TRCN0000024617 | Dtymk | 1.21 | 1.03 | 1.18 |
| TRCN0000024618 | Dtymk | 1.11 | 1.06 | 1.05 |
| TRCN0000012652 | Chek1 | 0.70 | 0.30 | 2.31 |
| TRCN0000012651 | Chek1 | 1.57 | 0.73 | 2.14 |
| TRCN0000012648 | Chek1 | 0.45 | 0.27 | 1.64 |
| TRCN0000012649 | Chek1 | 1.07 | 0.85 | 1.26 |
| TRCN0000012650 | Chek1 | 1.15 | 0.96 | 1.20 |
| TRCN0000041829 | Pdhb | 0.90 | 0.51 | 1.77 |
| TRCN0000041832 | Pdhb | 1.14 | 0.70 | 1.62 |
| TRCN0000041828 | Pdhb | 1.42 | 0.94 | 1.52 |
| TRCN0000041831 | Pdhb | 1.00 | 0.90 | 1.12 |
| TRCN0000041830 | Pdhb | 1.19 | 1.12 | 1.06 |
| TRCN0000025400 | Cmpk1 | 1.26 | 0.90 | 1.40 |
| TRCN0000025401 | Cmpk1 | 1.32 | 1.02 | 1.30 |
| TRCN0000025403 | Cmpk1 | 1.18 | 0.91 | 1.29 |
| TRCN0000025399 | Cmpk1 | 1.69 | 1.33 | 1.26 |
| TRCN0000025402 | Cmpk1 | 1.44 | 1.15 | 1.26 |
| TRCN0000103536 | Kdm4b | 1.59 | 1.01 | 1.57 |
| TRCN0000103539 | Kdm4b | 1.31 | 1.00 | 1.31 |
| TRCN0000103535 | Kdm4b | 1.25 | 1.03 | 1.21 |
| TRCN0000103537 | Kdm4b | 1.14 | 0.95 | 1.21 |
| TRCN0000103538 | Kdm4b | 1.23 | 1.19 | 1.03 |
| TRCN0000082029 | Med10 | 1.27 | 0.83 | 1.53 |
| TRCN0000082031 | Med10 | 1.36 | 1.07 | 1.28 |
| TRCN0000082030 | Med10 | 0.93 | 0.76 | 1.22 |
| TRCN0000082028 | Med10 | 1.18 | 1.04 | 1.14 |
| TRCN0000082032 | Med10 | 0.99 | 1.03 | 0.96 |
| TRCN0000096478 | Ncor1 | 1.06 | 0.73 | 1.46 |
| TRCN0000096475 | Ncor1 | 1.30 | 0.99 | 1.31 |
| TRCN0000096477 | Ncor1 | 1.30 | 1.14 | 1.14 |
| TRCN0000096474 | Ncor1 | 1.42 | 1.25 | 1.13 |
| TRCN0000096476 | Ncor1 | 1.51 | 1.69 | 0.89 |
| TRCN0000009730 | Bnip3l | 1.33 | 0.96 | 1.38 |
| TRCN0000009732 | Bnip3l | 1.33 | 1.00 | 1.33 |
| TRCN0000009733 | Bnip3l | 1.01 | 0.85 | 1.20 |
| TRCN0000009729 | Bnip3l | 1.46 | 1.31 | 1.11 |
| TRCN0000009731 | Bnip3l | 1.44 | 1.33 | 1.08 |
| TRCN0000094709 | Tspan33 | 1.37 | 1.00 | 1.37 |
| TRCN0000094712 | Tspan33 | 1.13 | 0.86 | 1.32 |
| TRCN0000094713 | Tspan33 | 1.18 | 1.02 | 1.16 |
| TRCN0000094711 | Tspan33 | 1.09 | 0.97 | 1.12 |
| TRCN0000023516 | Ntrk3 | 1.32 | 0.97 | 1.36 |
| TRCN0000023514 | Ntrk3 | 1.28 | 1.00 | 1.28 |
| TRCN0000023515 | Ntrk3 | 1.12 | 0.95 | 1.18 |
| TRCN0000023518 | Ntrk3 | 1.11 | 1.16 | 0.95 |
| TRCN0000023517 | Ntrk3 | 1.06 | 1.14 | 0.93 |
| TRCN0000104619 | Calm4 | 1.44 | 1.06 | 1.35 |
| TRCN0000104617 | Calm4 | 1.26 | 0.94 | 1.35 |
| TRCN0000104616 | Calm4 | 1.03 | 0.84 | 1.23 |
| TRCN0000104618 | Calm4 | 1.04 | 1.01 | 1.03 |
| TRCN0000104615 | Calm4 | 0.84 | 0.84 | 1.00 |
| TRCN0000103560 | Foxj1 | 1.39 | 1.04 | 1.34 |
| TRCN0000103562 | Foxj1 | 1.03 | 0.80 | 1.30 |
| TRCN0000103564 | Foxj1 | 0.69 | 0.67 | 1.03 |
| TRCN0000103561 | Foxj1 | 1.21 | 1.21 | 0.99 |
| TRCN0000103563 | Foxj1 | 1.02 | 1.06 | 0.96 |
| TRCN0000071292 | Ptges3 | 9.70 | 0.54 | 1.30 |
| TRCN0000071291 | Ptges3 | 1.24 | 0.97 | 1.28 |
| TRCN0000071288 | Ptges3 | 1.08 | 1.06 | 1.02 |
| TRCN0000071290 | Ptges3 | 1.25 | 1.50 | 0.83 |

BOLD = Values >2 SDs above the average of the control hairpins
Control Average 1.03
SD 0.12

TABLE 6
shRNAs used in experiments

| | | | | | Hairpin- | Growth of | |
|---|---|---|---|---|---|---|---|
| | | | | | rank in | Lkb1-wt/Lkb1- | |
| | | | SEQ | | pooled | null in | mRNA |
| Hairpin | | | ID | | shRNA | array-based | remaining |
| name | Hairpin ID | Hairpin Sequence | NO: | Target gene | screen | validation | (by qPCR) |
| shDtymk-1 | TRCN0000024614 | CAAGCTTCTGAATTCCTACTT | 13 | Mouse Dtymk | 27050 | 4.16 | 20.00% |
| shDtymk-2 | TRCN0000024617 | CTTCTCTGCAAACCGCTGGGA | 14 | Mouse Dtymk | 19621 | 1.18 | 66.70% |
| shDtymk-3 | TRCN0000024616 | ACGGAACTAGAGGATCACTCT | 15 | Mouse Dtymk | 100 | 2.96 | 27.80% |
| shDtymk-4 | TRCN0000024615 | GAAATCGGCAAGCTTCTGAAT | 16 | Mouse Dtymk | 28546 | 1.67 | 51.10% |
| shDtymk-5 | TRCN0000024618 | CAAGACCACGCAGGCCCTCAA | 17 | Mouse Dtymk | 5760 | 1.05 | 77.90% |
| shChek1-1 | TRCN0000012651 | GCTGTGAATAGAATAACTGAA | 18 | Mouse Chek1 | 327 | 2.14 | 35.50% |
| shChek1-2 | TRCN0000012649 | GCCACGAGAATGTAGTGAAAT | 19 | Mouse Chek1 | 14869 | 1.26 | 43.90% |
| shChek1-3 | TRCN0000012648 | CCCATGTAGTAGTATCACTTT | 20 | Mouse Chek1 | 23815 | 1.64 | 42.10% |
| shChek1-4 | TRCN0000012652 | GTGGAAGAAGAGTTGTATGAA | 21 | Mouse Chek1 | 26693 | 2.31 | 30.20% |
| shChek1-5 | TRCN0000012650 | GCAACGGTATTTCGGCATAAT | 22 | Mouse Chek1 | 23858 | 1.2 | 40.70% |
| shDTYMK-D3 | TRCP0003962305 | GTCCTGTTCCTCCAGTTACAG | 23 | Human DTYMK | | | |
| shDTYMK-D8 | TRCN0000024615 | GAAATCGGCAAGCTTCTGAAT | 24 | Human DTYMK | | | |
| shDTYMK-D10 | TRCN0000024618 | CAAGACCACGCAGGGCCTCAA | 25 | Human DTYMK | | | |
| shGFP | TRCN0000207065 | GCGATCACATGGTCCTGCTGG | 26 | None | | | |

### REFERENCES

1. Arner, E. S., and Eriksson, S. (1995). Mammalian deoxyribonucleoside kinases. Pharmacol Ther 67, 155-186.
2. Avizienyte, E., Loukola, A., Roth, S., Hemminki, A., Tarkkanen, M., Salovaara, R., Arola, J., Butzow, R., Husgafvel-Pursiainen, K., Kokkola, A., et al. (1999). LKB1 somatic mutations in sporadic tumors. Am J Pathol 154, 677-681.
3. Ayusawa, D., Shimizu, K., Koyama, H., Takeishi, K., and Seno, T. (1983). Accumulation of DNA strand breaks during thymineless death in thymidylate synthase-negative mutants of mouse FM3A cells. J Biol Chem 258, 12448-12454.
4. Barbie, D. A., Tamayo, P., Boehm, J. S., Kim, S. Y., Moody, S. E., Dunn, I. F., Schinzel, A. C., Sandy, P., Meylan, E., Scholl, C., et al. (2009). Systematic RNA interference reveals that oncogenic KRAS-driven cancers require TBK1. Nature 462, 108-112.
5. Bartek, J., and Lukas, J. (2003). Chk1 and Chk2 kinases in checkpoint control and cancer. Cancer Cell 3, 421-429.
6. Begg, A. C., Stewart, F. A., and Vens, C. (2011). Strategies to improve radiotherapy with targeted drugs. Nat Rev Cancer 11, 239-253.
7. Bender, A., and Pringle, J. R. (1991). Use of a screen for synthetic lethal and multicopy suppressee mutants to identify two new genes involved in morphogenesis in Saccharomyces cerevisiae. Mol Cell Biol 11, 1295-1305.
8. Bester, A. C., Roniger, M., Oren, Y. S., Im, M. M., Sarni, D., Chaoat, M., Bensimon, A., Zamir, G., Shewach, D. S., and Kerem, B. (2011). Nucleotide deficiency promotes genomic instability in early stages of cancer development. Cell 145, 435-446.
9. Blount, B. C., Mack, M. M., Wehr, C. M., MacGregor, J. T., Hiatt, R. A., Wang, G., Wickramasinghe, S. N., Everson, R. B., and Ames, B. N. (1997). Folate deficiency causes uracil misincorporation into human DNA and chromosome breakage: implications for cancer and neuronal damage. Proc Natl Acad Sci USA 94, 3290-3295.
10. Carrassa, L., and Damia, G. (2011). Unleashing Chk1 in cancer therapy. Cell Cycle 10, 2121-2128.
11. Ding, L., Getz, G., Wheeler, D. A., Mardis, E. R., McLellan, M. D., Cibulskis, K., Sougnez, C., Greulich, H., Muzny, D. M., Morgan, M. B., et al. (2008). Somatic mutations affect key pathways in lung adenocarcinoma. Nature 455, 1069-1075.
12. Faivre, S., Kroemer, G., and Raymond, E. (2006). Current development of mTOR inhibitors as anticancer agents. Nat Rev Drug Discov 5, 671-688.
13. Feldenberg, L. R., Thevananther, S., del Rio, M., de Leon, M., and Devarajan, P. (1999). Partial ATP depletion induces Fas- and caspase-mediated apoptosis in MDCK cells. Am J Physiol 276, F837-846.
14. Fuhrer, T., Heer, D., Begemann, B., and Zamboni, N. (2011). High-throughput, accurate mass metabolome profiling of cellular extracts by flow injection-time-of-flight mass spectrometry. Anal Chem 83, 7074-7080.
15. Gilad, 0., Nabet, B. Y., Ragland, R. L., Schoppy, D. W., Smith, K. D., Durham, A. C., and Brown, E. J. (2010). Combining ATR suppression with oncogenic Ras synergistically increases genomic instability, causing synthetic lethality or tumorigenesis in a dosage-dependent manner. Cancer Res 70, 9693-9702.
16. Gurumurthy, S., Xie, S. Z., Alagesan, B., Kim, J., Yusuf, R. Z., Saez, B., Tzatsos, A., Ozsolak, F., Milos, P., Ferrari, F., et al. (2010). The Lkb1 metabolic sensor maintains haematopoietic stem cell survival. Nature 468, 659-663.
17. Hardie, D. G. (2007). AMP-activated/SNF1 protein kinases: conserved guardians of cellular energy. Nat Rev Mol Cell Biol 8, 774-785.
18. Hartman, J. L. t., Garvik, B., and Hartwell, L. (2001). Principles for the buffering of genetic variation. Science 291, 1001-1004.
19. Hearle, N., Schumacher, V., Menko, F. H., Olschwang, S., Boardman, L. A., Gille, J. J., Keller, J. J., Westerman, A. M., Scott, R. J., Lim, W., et al. (2006). Frequency and spectrum of cancers in the Peutz-Jeghers syndrome. Clin Cancer Res 12, 3209-3215.
20. Hemminki, A., Markie, D., Tomlinson, I., Avizienyte, E., Roth, S., Loukola, A., Bignell, G., Warren, W., Aminoff, M., Hoglund, P., et al. (1998). A serine/threonine kinase gene defective in Peutz-Jeghers syndrome. Nature 391, 184-187.
21. Huang, S. H., Tang, A., Drisco, B., Zhang, S. Q., Seeger, R., Li, C., and Jong, A. (1994). Human dTMP kinase: gene expression and enzymatic activity coinciding with cell cycle progression and cell growth. DNA Cell Biol 13, 461-471.
22. Jansen, M., Ten Klooster, J. P., Offerhaus, G. J., and Clevers, H. (2009). LKB1 and AMPK family signaling: the intimate link between cell polarity and energy metabolism. Physiol Rev 89, 777-798.
23. Ji, H., Ramsey, M. R., Hayes, D. N., Fan, C., McNamara, K., Kozlowski, P., Torrice, C., Wu, M. C., Shimamura, T., Perera, S. A., et al. (2007). LKB1 modulates lung cancer differentiation and metastasis. Nature 448, 807-810.
24. Liu, Y., Parry, J. A., Chin, A., Duensing, S., and Duensing, A. (2008). Soluble histone H2AX is induced by DNA replication stress and sensitizes cells to undergo apoptosis. Mol Cancer 7, 61.
25. Luo, B., Cheung, H. W., Subramanian, A., Sharifnia, T., Okamoto, M., Yang, X., Hinkle, G., Boehm, J. S., Beroukhim, R., Weir, B. A., et al. (2008). Highly parallel identification of essential genes in cancer cells. Proc Natl Acad Sci USA 105, 20380-20385.
26. Luo, J., Emanuele, M. J., Li, D., Creighton, C. J., Schlabach, M. R., Westbrook, T. F., Wong, K. K., and Elledge, S. J. (2009). A genome-wide RNAi screen identifies multiple synthetic lethal interactions with the Ras oncogene. Cell 137, 835-848.
27. Makowski, L., and Hayes, D. N. (2008). Role of LKB1 in lung cancer development. Br J Cancer 99, 683-688.
28. Matsumoto, S., Iwakawa, R., Takahashi, K., Kohno, T., Nakanishi, Y., Matsuno, Y., Suzuki, K., Nakamoto, M., Shimizu, E., Minna, J. D., et al. (2007). Prevalence and specificity of LKB1 genetic alterations in lung cancers. Oncogene 26, 5911-5918.
29. Mogi, A., and Kuwano, H. (2011). TP53 mutations in nonsmall cell lung cancer. J Biomed Biotechnol 2011, 583929.
30. Reichard, P. (1988). Interactions between deoxyribonucleotide and DNA synthesis. Annu Rev Biochem 57, 349-374.
31. Rogakou, E. P., Boon, C., Redon, C., and Bonner, W. M. (1999). Megabase chromatin domains involved in DNA double-strand breaks in vivo. J Cell Biol 146, 905-916.
32. Sanchez-Cespedes, M., Parrella, P., Esteller, M., Nomoto, S., Trink, B, Engles, J. M., Westra, W. H., Herman, J. G., and Sidransky, D. (2002). Inactivation of LKB1/STK11 is a common event in adenocarcinomas of the lung. Cancer Res 62, 3659-3662.
33. Segurado, M., and Diffley, J. F. (2008). Separate roles for the DNA damage checkpoint protein kinases in stabilizing DNA replication forks. Genes Dev 22, 1816-1827.
34. Shah, U., Sharpless, N. E., and Hayes, D. N. (2008). LKB1 and lung cancer: more than the usual suspects. Cancer Res 68, 3562-3565.
35. Shaw, R. J., Kosmatka, M., Bardeesy, N., Hurley, R. L., Witters, L. A., DePinho, R. A., and Cantley, L. C. (2004). The tumor suppressor LKB1 kinase directly activates AMP-activated kinase and regulates apoptosis in response to energy stress. Proc Natl Acad Sci USA 101, 3329-3335.
36. Syljuasen, R. G., Sorensen, C. S., Hansen, L. T., Fugger, K., Lundin, C., Johansson, F., Helleday, T., Sehested, M., Lukas, J., and Bartek, J. (2005). Inhibition of human Chk1 causes increased initiation of DNA replication, phosphorylation of ATR targets, and DNA breakage. Mol Cell Biol 25, 3553-3562.
37. Tse, A. N., Rendahl, K. G., Sheikh, T., Cheema, H., Aardalen, K., Embry, M., Ma, S., Moler, E. J., Ni, Z. J., Lopes de Menezes, D. E., et al. (2007). CHIR-124, a novel potent inhibitor of Chk1, potentiates the cytotoxicity of topoisomerase I poisons in vitro and in vivo. Clin Cancer Res 13, 591-602.
38. Van Triest, B., Pinedo, H. M., Giaccone, G., and Peters, G. J. (2000). Downstream molecular determinants of response to 5-fluorouracil and antifolate thymidylate synthase inhibitors. Ann Oncol 11, 385-391.
39. Whitehurst, A. W., Bodemann, B. O., Cardenas, J., Ferguson, D., Girard, L., Peyton, M., Minna, J. D., Michnoff, C., Hao, W., Roth, M. G., et al. (2007). Synthetic lethal screen identification of chemosensitizer loci in cancer cells. Nature 446, 815-819.
40. Xiao, Z., Chen, Z., Gunasekera, A. H., Sowin, T. J., Rosenberg, S. H., Fesik, S., and Zhang, H. (2003). Chk1 mediates S and G2 arrests through Cdc25A degradation in response to DNA-damaging agents. J Biol Chem 278, 21767-21773.
41. Zabludoff, S. D., Deng, C., Grondine, M. R., Sheehy, A. M., Ashwell, S., Caleb, B. L., Green, S., Haye, H. R., Horn, C. L., Janetka, J. W., et al. (2008). AZD7762, a novel checkpoint kinase inhibitor, drives checkpoint abrogation and potentiates DNA-targeted therapies. Mol Cancer Ther 7, 2955-2966.
42. Zhao, H., Watkins, J. L., and Piwnica-Worms, H. (2002). Disruption of the checkpoint kinase 1/cell division cycle 25A pathway abrogates ionizing radiation-induced S and G2 checkpoints. Proc Natl Acad Sci USA 99, 14795-14800.
43. Fuhrer, T., Heer, D., Begemann, B., and Zamboni, N. (2011). High-throughput, accurate mass metabolome profiling of cellular extracts by flow injection-time-of-flight mass spectrometry. Anal Chem 83, 7074-7080.
44. Luo, B., Cheung, H. W., Subramanian, A., Sharifnia, T., Okamoto, M., Yang, X., Hinkle, G., Boehm, J. S., Beroukhim, R., Weir, B. A., et al. (2008). Highly parallel identification of essential genes in cancer cells. Proc Natl Acad Sci USA 105, 20380-20385.
45. Moffat, J., Grueneberg, D. A., Yang, X., Kim, S. Y., Kloepfer, A. M., Hinkle, G., Piqani, B., Eisenhaure, T. M., Luo, B., Grenier, J. K., et al. (2006). A lentiviral RNAi library for human and mouse genes applied to an arrayed viral high-content screen. Cell 124, 1283-1298.

1. A method of treating a subject having a Lkb1 null cancer comprising
administering to said subject a compound that inhibits the expression of activity of deoxythymidylate kinase (DTYMK), checkpoint kinase 1 (CHEK1) or both.

2. The method of claim 1, wherein said cancer is lung cancer, melanoma, pancreatic cancer, endometrial cancer or ovarian cancer.

3. The method of claim 1, wherein the compound is a nucleic acid, an antibody or a small molecule.

4. The method of claim 1, wherein the compound is a CHEK1 inhibitor.

5. The method of claim 4, wherein the CHEK 1 inhibitor is AZD7762, Go-6976, UCN-01, CCT244747, TCS2312, PD 407824, PF 477736, PD-321852,
SB218078, LY2603618, LY2606368, CEP-3891, SAR-020106,
debromohymenialdisine, or CHIR24.

6. The method of claim 1, further comprising
administering a chemotherapeutic agent.

7. The method of claim 6, wherein the chemotherapeutic agent is a tyrosine kinase inhibitor or an mTOR inhibitor.

8. A method of screening for therapeutic targets for treating cancer comprising: a. providing a cell that is null for a Lkb1 gene, an ATM gene, a TSC1 gene, a PTEN gene or a Notch gene;
b. contacting the cell with a library of RNAi; and
c. identifying an RNAi which is lethal to said cell;
thereby identifying a therapeutic target for treating cancer.

9. A method of treating an ATM, a TSC1, a PTEN, or a Notch null cancer comprising administering to said subject a compound that inhibits the expression of activity of the therapeutic target identified in claim 8.

10. A cell expressing KRAS G12D and comprising a disruption of the Trp53 gene, the Lkb1 gene or both, wherein the disruption results in decreased expression or activity of the Trp53 gene, the Lkb1 gene or both in the cell.

11. The cell of claim 10, wherein said cell is a cancer cell.

12. The cell of claim 11, wherein said cancer cell is a lung cancer cell, a melanoma cancer cell, a pancreatic cancer cell, an endometrial cancer cell or an ovarian cancer cell.
